(19) United States
(12) Patent Application Publication
(10) Pub. No.: US 2008/0306333 A1
(43) Pub. Date: Dec. 11, 2008

(54) Apparatus and Method for Endoscopic Surgical Procedures

(76) Inventor: Albert K. Chin, Palo Alto, CA (US)

Correspondence Address: FENWICK & WEST LLP, SILICON VALLEY CENTER, 801 CALIFORNIA STREET, MOUNTAIN VIEW, CA, 94041, US

(21) Appl. No.: 11/747,356

(22) Filed: May 11, 2007

Related U.S. Application Data
(62) Division of application No. 10/347,212, filed on Jan. 17, 2003, now abandoned.
Application No. 10/347,212 is a continuation-in-part of application No. 10/174,454, filed on Jun. 17, 2002.
Application No. 10/174,454 is a continuation-in-part of application No. 10/140,309, filed on May 6, 2002.
Application No. 10/140,309 is a continuation of application No. 09/635,721, filed on Aug. 9, 2000.
Application No. 09/635,721 is a continuation-in-part of application No. 09/779,715, filed on Feb. 8, 2001, now Pat. No. 6,569,082.
Application No. 09/779,715 is a continuation of application No. 09/738,608, filed on Dec. 14, 2000, now abandoned.
Application No. 09/738,608 is a continuation-in-part of application No. 09/635,345, filed on Aug. 9, 2000, now Pat. No. 7,398,781.
Application No. 09/635,345 is a continuation-in-part of application No. 10/006,321, filed on Dec. 4, 2001, now Pat. No. 6,706,052.
Application No. 10/006,321 is a division of application No. 09/915,695, filed on Jul. 25, 2001, now Pat. No. 6,428,556.
(60) Provisional application No. 60/148,130, filed on Aug. 10, 1999.
(60) Provisional application No. 60/150,737, filed on Aug. 25, 1999.

(51) Int. Cl. A61B 1/018; A61B 17/04; A61B 18/18; A61B 18/20; A61H 1/00

(52) U.S. Cl. 600/104; 606/142; 606/148; 606/41; 606/14; 601/2

(57) ABSTRACT

Apparatus and method for performing surgical procedures within the mediastinum and within the pericardium include an endoscopic cannula having a transparent tip, and an endoscope for introduction into the mediastinum and optionally into the pericardium via a single subxiphoid incision. A cavity may be initially dilated for advancing the endoscopic cannula using a dilating tool that exerts a lateral-expansive force against surrounding tissue for evaluating the endoscopic cannula to be introduced into the mediastinum. Other surgical instruments are positioned through the endoscopic cannula to cut a flap of the pericardium as an opening through which other surgical apparatus may be introduced. The endoscopic cannula may be swept around selected regions of the heart through an aperture near the apex of the heart to facilitate placement of epicardial tacks about regions of the heart.

### CROSS-REFERENCES TO RELATED APPLICATIONS

This is a divisional application of pending application Ser. No. 10/347,212, entitled “Apparatus and Methods for Endoscopic Surgical Procedures,” filed on Jan. 17, 2003, which in turn is a continuation-in-part application of pending application Ser. No. 10/174,454, entitled “Releasable Guide And Method For Endoscopic Cardiac Lead Placement”, filed on Jun. 17, 2002, which is a continuation-in-part of pending application Ser. No. 10/140,309 entitled “Methods And Apparatus For Endoscopic Cardiac Surgery”, filed on May 6, 2002, which is a continuation of pending application Ser. No. 09/635,721 entitled “Apparatus For Endoscopic Access”, filed on Aug. 9, 2000, which claims the benefit of provisional applications Ser. No. 60/148,130 filed on Aug. 10, 1999 and Ser. No. 60/150,737 filed on Aug. 25, 1999. This application is also a continuation-in-part application of the U.S. Pat. No. 6,569,082, entitled “Apparatus And Methods For Cardiac Restraint”, filed on Feb. 8, 2001 which is a continuation of abandoned application Ser. No. 09/738,608 entitled “Apparatus And Methods For Cardiac Restraint”, filed on Dec. 14, 2000, which is a continuation-in-part of pending application Ser. No. 09/635,345, entitled “Apparatus And Method For Subxiphoid Endoscopic Access”, filed on Aug. 9, 2000, which claims the benefit of the aforecited provisional applications. This application is also a continuation-in-part of U.S. Pat. No. 6,706,052 entitled “Longitudinal Dilator and Method”, issued on Mar. 16, 2004, which is a continuation of U.S. Pat. No. 6,428,556 entitled “Longitudinal Dilator And Method”, issued on Aug. 6, 2002, which claims the benefit of the aforecited provisional application Ser. No. 60/150,737, filed on Aug. 25, 1999, which applications are incorporated herein in their entireties by these references to form a part hereof.

### FIELD OF THE INVENTION

This invention relates to apparatus and methods for performing minimally invasive surgery, and more particularly to endoscopic subxiphoid surgical procedures for accessing the mediastinum and the pericardium for various surgical remediations via closed-chest surgical methods, and to access all regions of the heart, for example, to install conductive wires, ablate tissue and to attach heart supports and constraints for inhibiting cardiac distention.

### BACKGROUND OF THE INVENTION

Several different incisions have traditionally been used to access mediastinal organs, such as the heart (surrounded by the pericardium), the esophagus, and lymphatic glands. Examples of such incisions are sternotomy (a division of the patient's sternum), thoracotomy (an incision between two adjacent ribs), and a large subxiphoid incision to create a pericardial window by exposing and excising a portion of the pericardium. For example, a subxiphoid incision has been made to allow excision of the xiphoid, and retraction of the sternum upward to expose the anterior pericardium.

These procedures, however, are all quite invasive, requiring large incisions or open heart surgery. Thoracotomy is additionally invasive as it requires the deflation of one or both lungs, since the approach is via the pleural cavity. Nevertheless, when it is desirable to access other regions of the heart than merely its anterior region, the current practice is to employ these invasive methods to dislodge the heart from its resting place within the pericardium, so that all regions of the heart may be accessed and cardiac procedures performed. For example, to access both left and right sides of the heart, as well as the posterior and anterior regions, surgeons are currently using a partial or full stemotomy (i.e. a partial or full division of the patient's sternum) to gain access to the several regions of the heart by permitting the heart to be rotated or lifted out of its resting place in the chest. Such a procedure, however, is too invasive, and thus not desirable.

With the advent of minimally invasive surgery, approaches have been developed using smaller access incisions or ports. Coronary bypass surgery has been performed on the beating heart through direct incisions in the chest and abdomen, including sternotomies and thoracotomies. A subxiphoid incision has been used to anastomose a gastroepiploic artery to the posterior descending coronary artery for coronary artery bypass. These procedures, however, have been performed under direct vision, and thus still require a fairly large incision to assist the surgeon in observing the field of surgery.

To achieve even less invasive surgery, it is desirable to perform cardiac procedures endoscopically. Endoscopic coronary bypass surgery has been performed on a stopped heart following the institution of cardiopulmonary bypass. In this procedure, ports are placed in the intercostal spaces, through the chest wall, to allow placement of the endoscope and operating instruments. This method, however, does not enable the surgeon to access all regions of the heart. With port access surgery or beating heart surgery from a limited thoracotomy, only one side of the heart is accessible. For example, with a left thoracotomy or the introduction of left side ports, surgery is limited only to the left side of the heart. Endoscopic harvesting of the gastroepiploic artery for coronary artery bypass surgery has also been described, involving standard laparoscopic techniques of gas insufflation and introduction of laparoscopic forceps, scissors, and staplers. However, none of these minimally invasive methods allow access to all regions of the heart. Thus, a method and apparatus are needed to allow safe and minimally invasive access to all regions of the heart for performing cardiac procedures.

In addition, conventional procedures such as open heart surgery, port-access surgery using trocar ports and an endoscope, or beating heart surgery through a partial sternotomy or thoracotomy, all require making a large incision in the pericardium to expose the heart. Conventional methods of accessing the heart to perform cardiac procedures involve making an incision in the pericardium using a sharp-edged instrument through an incision in the chest. As the heart typically underlies the pericardium contiguously, the surgeon is presented with the difficult task of incising the pericardium without accidentally cutting the heart. To avoid this difficulty during port-access surgery, a second incision into the skin is also required to allow the insertion of forceps to pull the pericardium away from the heart. This allows the incision of the pericardium to be executed more safely. However, this technique requires multiple incisions in the patient and requires the advancement of multiple instruments in separate passageways to the pericardium.

In addition to requiring several incisions, the conventional techniques also typically require the incision in the pericardium to be lengthy. The sharp-edged instrument must slice a cut of sufficient length to allow the insertion of other surgical tools into the pericardium. At the end of the cardiac procedure, it is desirable to close the pericardial incision if possible, to reduce fibrous adhesions to the heart and pericarditis. With endoscopic post-access surgery, a long pericardial incision is difficult to close, due to the complexity of endoscopic suturing.

Another problem arising in conventional cardiac procedures is the dissection of a working tunnel from the initial incision to the pericardium. Mechanical probing of heart tissue may cause severe or dangerous cardiac arrhythmias such as ventricular fibrillation. Therefore, it is desirable to use a small dilating instrument to create the initial tunnel. However, the instruments currently available to perform cardiac procedures are typically large, and therefore a larger cavity must be dissected to allow these instruments to pass through to the pericardium. Although using a larger dilator may create the necessary space, a larger dilator may cause damage to the heart by causing cardiac arrhythmias as discussed above. If a small dilator is used to minimize this potential trauma, the working cavity may not be large enough to allow the larger instruments required in the procedure to be advanced to the pericardium. A further problem with conventional dilators such as balloon dissectors is that such tools exert shear force on the surrounding tissue as they are advanced in the body. Shear force has a tendency of causing vessel avulsion and tissue abrasion.

Various other schemes and devices have been previously devised in an attempt to enter the pericardium via a small portal of entry, or via a percutaneous puncture site. None of these systems permit reliable, safe entry under direct endoscopic visualization. U.S. Pat. No. 5,931,810 (Grabek) describes a grasping instrument with jaws that grasp the pericardium followed by advancement of a needle through a bore in the shaft of the instrument. The needle extends between the closed jaws of the device, into the pericardium. This concept suffers from unreliability, as it is difficult to ensure that the needle will pierce between two layers of pericardium that are compressed by the jaws of the device, without an active technique of holding the two opposed layers of pericardium apart. Thus, as there is no central cavity in a flap of pericardium grasped by the instrument jaws, a needle advanced down a central bore of the instrument may easily end up outside the pericardium, or embedded in the pericardium, instead of lying between the two layers of pericardium pinched together by the jaws. Also, axial advancement of the needle carries the potential of myocardial puncture. Needle entry with the Grabek device must be verified by subsequent passage of a guidewire into the pericardial sac, or by infusion of fluid or contrast material through the needle into the pericardial cavity.

U.S. Pat. No. 5,827,216 (Igo et al.) and U.S. Pat. No. 5,972,013 (Schmidt) both describe tubes that are placed in contact with the pericardium, applying a vacuum to pull a bleb of tissue into the tube, followed by penetration of the pericardial bleb with a needle. These techniques are unreliable, because there is generally a layer of fatty tissue adherent to the pericardial surface, and suction may pull fat into the tube instead of pericardium.

U.S. Pat. No. 5,071,428 (Chin et al.) describes a clamp with distal points that grasp a flap of pericardium, allowing a guidewire to be advanced within tubular guides to puncture through the pericardium. A tube may follow the guidewire into the intra pericardial space. The multiple steps of pericardial grasping, pericardial puncture, guidewire advancement, and catheter insertion render this technique less practical.

Apparatus and methods are needed to provide safe and minimally invasive access to all regions of the heart during cardiac procedures, requiring a minimum number of incisions, and without requiring a long incision either for initial access or at the pericardium.

One minimally-invasive surgical procedure accesses the heart to restrain the cardiac wall for the prevention or reduction of cardiac dilation in patients known to have experienced such dilation or who have a predisposition for such dilation occurring in the future. A cardiac restraint apparatus is typically applied to the epicardial surface of the heart to partially enclose the heart.

Cardiac dilation can result from such cardiac diseases as congestive heart disease, post-myocardial infarctions, dilated cardiomyopathy, and viral infections. In such cases, the heart may enlarge to such an extent that the adverse consequences of heart enlargement continue following recovery from the initial affliction with debilitating effect. In some cases, such as post-myocardial infarction, the dilation may be localized to only a portion of the heart. In other cases, such as hypertrophic cardiomyopathy, there is typically increased resistance to filling of the left ventricle with concomitant dilation of the left artia. In dilated cardiomyopathy, the dilation is typically of the left ventricle with resultant failure of the heart as a pump. In advanced cases, dilated cardiomyopathy involves the majority of the heart. Causes of congestive heart disease are not fully known.

As the heart enlarges, the heart is performing an increasing amount of work in order to pump blood during each heart beat. In time, the heart becomes so enlarged that the heart cannot adequately supply blood. An afflicted patient is fatigued, unable to perform even simple exerting tasks and experiences pain and discomfort. Further, as the heart enlarges, the internal heart valves cannot adequately close. This impairs the function of the valves and further reduces the heart's ability to supply blood. With each type of cardiac dilation, there are associated problems ranging from arrhythmias resulting from increased stretching of myocardial cells, to leakage of the cardiac valves due to enlargement of the valvular annulus.

Drugs are sometimes employed to assist in treating problems associated with cardiac dilation. For example, Digoxin increases the contractility of the cardiac muscle and thereby causes enhanced emptying of the dilated cardiac chambers. On the other hand, some drugs, for example, beta-blocking drugs, decrease the contractility of the heart and thus increase the likelihood of dilation. Other drugs, including angiotensin-converting enzyme inhibitors such as Enalopril, help to reduce the tendency of the heart to dilate under the increased diastolic pressure experienced when the contractility of the heart muscle decreases. Many of these drugs, however, have side effects which make them undesirable for long-term use.

Apparatus to prevent or reduce dilation and thereby reduce the consequences of dilation have also been described. Patches made from low porosity materials, for example Dacron™, have been used to support the cardiac wall. Other apparatus for similar purposes are described in the literature (see, for example U.S. Pat. Nos. 4,957,477; 5,131,905; 5,150,706; 5,143,082; 5,256,132; 5,702,343; 6,077,218; 6,085,754; 6,095,968).

The '477 patent discloses a double-walled jacket surrounding the heart. A fluid fills a chamber between the walls of the jacket. The inner wall is positioned against the heart and is pliable to move with the heart. Movement of the heart during beating displaces fluid within the jacket chamber. The '706 patent discloses a medical apparatus for enclosing an internal body organ, comprising a filamentary strand with noose and free end portions and a surgical bag with an opening. The '082 patent discloses a cooling net for cardiac or transplant surgery, comprising a porous net that is fitted and secured around the organ. Both of the '905 and '132 patents disclose cardiac assist apparatus which pump fluid into chambers opposing the heart to assist systolic contractions of the heart. The '343 and '218 patents disclose adjustable jackets to constrain cardiac expansion during diastole. The '754 patent discloses a biologically compatible jacket adapted to be secured to the heart. The '968 patent discloses a viscous cardioplasty jacket for buttressing the ventricular heart walls.

However, none of these patents disclose a sheath to facilitate endoscopic introduction of the apparatus, or guide elements for positioning the cardiac restraint apparatus around the heart, and none of these patents disclose hollow guide tubes that permit an instrument to be advanced through such tubes to engage the mouth of the jacket and secure the mouth of the jacket to the pericardium. Furthermore, none of these patents disclose introducing a cardiac restraint apparatus via a single subxiphoid incision. Accordingly, there is a need for an improved cardiac restraint apparatus that can be more easily introduced via minimally invasive surgical procedures.

In other minimally-invasive surgical procedures, undifferentiated satellite cells or myocytes or stem cells are injected into the myocardium of a beating heart in the endoscopic procedure of cellular cardiomyoplasty. This procedure is performed carefully to avoid complications using a specialized instrument, as described in the aforecited Related Applications, that is advanced through an operating channel of an endoscopic cannula to deliver cells in controlled manner into a beating heart. If a needle is used to inject the cells, sufficient control must be provided to ensure that the needle does not puncture a coronary vein or artery and cause hemorrhage within the pericardial space, with subsequent cardiac tamponade. Movement of the beating heart further complicates needle placement because of erratic movement of the coronary vessels as needle insertion is attempted. Similarly, placement of other elements such as epicardial pacing or defibrillation leads into the myocardium of a beating heart must be carefully placed to avoid puncture of a coronary vein or artery with concomitant complications.

In yet another minimally-invasive surgical procedure, ablation of tissue surrounding the pulmonary vein ostia at the site in the intrapericardial space where the veins enter into the left atrium is clinically recognized as a treatment for chronic atrial fibrillation. Cardiac surgeons have been entering the chest through a standard sternotomy, dissecting a tract under the superior vena cava and the inferior vena cava, and threading an ablation probe around the four pulmonary veins. The probe enters posterior to the superior vena cava, winds through the transverse sinus of the pericardium, loops around the four pulmonary veins, and exits the tract that was dissected posterior to the inferior vena cava. The tract formed posterior to the superior vena cava enters into the transverse sinus of the pericardium. The tract formed posterior to the inferior vena cava completes the path of the ablation probe around the pulmonary veins.

In order to perform the above described probe placement endoscopically, one endoscopic cannula is advanced through a thoracotomy incision, or other entry incision, into the intrapericardial space adjacent the superior vena cava, and a second endoscopic cannula is inserted into the right pleural cavity via another thoracotomy incision. This latter endoscopic cannula in the right pleural cavity is used to dissect through the right medial pleura and the pericardium posterior to the superior vena cava, guided by transillumination light emitted by the other endoscopic cannula.

This technique uses two endoscopes, and two full sets of endoscopic equipment, including endoscope, video camera, light source, video monitor and light cable. The physical space occupied by two sets of endoscopic equipment is cumbersome in the operating room, and the expense is prohibitive to hospitals. Therefore, it is desirable to perform the procedure using one set of endoscopic equipment and one endoscopic cannula.

Various operative techniques have been suggested for repairing regurgitant mitral valves, including surgical placement of a closed or open ring at the mitral annulus to correct a dilated annulus causing regurgitation through the valve. A “bowtie” stitch placed across the mitral orifice may reform a large orifice into two smaller openings and decrease mitral regurgitation. Alternatively, intravascular repairs include insertion of a stent or spring into the coronary sinus to reshape the mitral annulus by placing such a preformed structure into the heart's venous system.

In congestive heart failure, cardiomegaly (enlargement of the heart) may be treated by an external elastic support device that corsets the heart. Expansion of the heart during diastole is constrained by a jacket that expands to a predetermined amount to prevent further distension. Other devices seek to reduce the wall tension in the heart by using tension members to draw the walls of a heart chamber toward each other. Devices of these types are described in the literature (see, for example, U.S. Pat. Nos. 5,702,343 and 6,332,863).

The cardiac jacket reinforcement device described has the advantage of enclosing the entire heart, while the tension members exert force on several different points on the heart. However, the jacket is difficult or impossible to place on the heart without opening the chest via a sternotomy or thoracotomy.

Dilation of tissue is important for many surgical procedures that may be performed endoscopically, including, for example, vessel harvesting and surgical access to the mediastinum. Tissue must be dilated to allow atraumatic advancement of surgical instruments within the body to a surgical site. To perform a vessel harvesting procedure, for example, to remove a segment of the saphenous vein for use as a graft vessel in cardiovascular surgery, a ligation tool, typically maintained within a cannula providing endoscopic visualization, must be advanced to a vessel of interest to ligate the ends of the vessel and any intermediate side branches. However, prior to advancing the ligation tool, the path to the end of the segment of the vessel must be created while creating as little trauma to the surrounding tissue as possible. Present systems used in endoscopic vessel harvesting incorporate a transparent tapered tip to dissect the saphenous vein from surrounding connective tissue. A previous system also dilated the peri-vascular cavity by serially inflating a short balloon along the length of the cavity. Mechanical means of dilating the cavity have also been described, for example, such as those described in U.S. Pat. No. 6,030,406, including moving arms or cams which expand outward upon activation of a sleeve or a trigger. In these embodiments, a balloon or active mechanical dilator of short length is used, because the short length ensures that the dilators will be able to generate an adequate amount of force to successfully dilate the tunnel. For example, it is known that a short angioplasty balloon generates greater dilating force than a long angioplasty balloon. The wall tension of an inflated balloon is responsible for generating the dilating force. The longitudinal wall of a long balloon maintains less tension in the middle area of the balloon. This area of less tension corresponds to a diminished dilating force. Thus, many surgeons prefer using short balloons because a short balloon can maintain tension across the entire body of the balloon. However, a short balloon or mechanical dilator in a tissue-dilating system must be activated multiple times along the length of the tunnel to achieve a complete expansion of the tunnel. This repeated motion may tire the hand of a surgeon performing the procedure, and, further, stepwise dilation may result in formation of an uneven tunnel, with an irregular inner contour. Therefore, an apparatus and method are needed that provide adequate tissue-dilating force, result in an even dilation, and not require multiple repeated movements to complete the dilation procedure.

### SUMMARY OF THE INVENTION

In accordance with the present invention, apparatus and methods for using the apparatus provide safe and minimally invasive access to mediastinal structures including the pericardium that surrounds the heart. More specifically, the apparatus and methods access the pericardium via a subxiphoid approach, access the heart within the pericardium, and facilitate performing cardiac procedures thereon.

The surgical apparatus for performing the surgical method in accordance with one embodiment of this invention is an endoscopic cannula comprising a cannula, a transparent tip located at the distal end of the cannula, and an endoscope positioned for visualization at the distal end of the cannula. The cannula has at least one endoscopic lumen and one or more additional instrument lumens for advancement of surgical instruments therethrough. The transparent tip is tapered to provide better visualization via the endoscope for dissecting and dilating tissue within the field of view. The transparent tip has a generally conical shape and may be removable and replaceable at the distal end of the cannula as desired to obtain clearer images of the surgical site.

In one embodiment, the endoscopic cannula comprises an access port positioned at a proximal end of the cannula for receiving surgical instruments into an instrument lumen of the cannula, and further comprises an endoscopic eyepiece that is skewed relative to the proximal end of the endoscope for facilitating the viewing of a surgical site through the endoscope while minimizing interference with surgical instruments introduced into the cannula.

In another embodiment, the cannula is articulable, and includes a wire positioned within a wire lumen in the cannula with a distal end attached to a distal end of the cannula. An articulating lever is positioned near the proximal end of the cannula attached to the proximal end of the wire for tensioning the wire in a first position to cause the distal end of the cannula to bend away from the elongated axis of the cannula, and for relaxing the wire in a second position to position the distal end of the cannula substantially aligned with the elongated axis of the cannula.

In accordance with one method embodiment of the present invention, the endoscopic cannula is either directly advanced to the mediastinum or alternatively, a cavity is first dilated and the endoscopic cannula is advanced through the dilated cavity. Once the endoscopic cannula is advanced into the mediastinum, surgical instruments are advanced through lumens of the cannula that therefore serve as access ports, and surgical procedures can be performed with the surgical instruments within the mediastinum. The endoscopic cannula may be inserted directly into an initial subxiphoid incision to be guided under endoscopic visualization to the surgical site. Alternatively, a cavity or channel may be dissected toward the surgical site and dilated using a dilation tool according to this invention, and the cannula may be subsequently advanced within the dilated cavity. The second method is advantageous because as the dilation tool generally has a smaller diameter than the endoscopic cannula, initially inserting the dilation tool minimizes tissue trauma and reduces the chance of ventricular fibrillation due to irritation of the heart upon contact therewith by a large diameter instrument.

The dilation tool optionally used to dilate a cavity for the endoscopic cannula has an elongated inner cannula with a transparent distal tip and an outer sheath that is expandable outwardly along the elongated axis. The dilation tool has a small maximal dimension which minimizes trauma to tissue surrounding the cavity and to the pericardium upon reaching the pericardium. The inner cannula has an enlarged tip positioned distal to the distal end of the outer expandable sheath. Withdrawing the enlarged tip on the inner cannula through the outer expandable sheath expands the sheath to dilate a cavity in the surrounding tissue. The expandable sheath exerts a radial force against the surrounding tissue as the enlarged tip is retracted through the sheath to promote less traumatic dilation than conventional dilation techniques in which shear force is directly applied to surrounding tissue.

Once the cavity is dilated, the endoscopic cannula is then inserted into the incision and advanced into the proximal end of the expandable sheath. Advancing the endoscopic cannula toward the pericardium through the sheath also causes the expandable sheath to expand further and dilate the cavity or channels to a sufficient size to accommodate the endoscopic cannula. The expandable sheath provides the additional benefit of guiding the endoscopic cannula to the proper position at the pericardium. Alternatively, the endoscopic cannula is inserted directly into and through an initial incision without dilation.

To perform cardiac procedures within the pericardium, an opening is formed in the pericardium for inserting the endoscopic cannula into the pericardium. A pericardial entry instrument in accordance with one embodiment of the present invention includes a grasping tool for gripping a portion of the pericardium, and a cutting tool slidably disposed on the outside of the grasping tool for cutting the gripped portion of the pericardium under endoscopic visualization. The pericardial entry instrument is advanced through a lumen of the endoscopic cannula toward the pericardium and is positioned to cut an opening into the pericardium for advancing other surgical instruments into the pericardium.

In particular, the pericardium entry instrument according to one embodiment of the present invention uses a tube to cut along a flap of pericardium grasped by jaws, under direct visualization. There is no ambiguity regarding success or failure of the pericardial entry, since the pericardial hole is observed as it occurs.

In one method embodiment of the present invention, the pericardial entry instrument is advanced tangentially to the pericardium to allow the grasping tool to grasp a flap of the pericardium without endangering the underlying heart. Once a flap of the pericardium is grasped, the cutting tool is extended to cut the flap, creating a small opening through which other surgical instruments may be introduced. In a preferred embodiment, the cutting tool is a tubular cutting device which creates a circular opening of small circumference for producing a correspondingly small opening in the pericardium.

One embodiment of a method of performing a cardiac procedure used in conjunction with the described apparatus comprises first making a single subxiphoid incision to provide initial access into the patient's body, inserting an endoscopic cannula into the incision, advancing the endoscopic cannula to the mediastinum under endoscopic visualization, and performing the surgical procedure within the mediastinum. Optionally, the method may include initially providing a dilated cavity in the manner as previously described for passing the endoscopic cannula into the mediastinum and performing the surgical procedure within the mediastinum.

The methods according to the present invention facilitates performing cardiac surgical procedures within the pericardium. For these procedures, the endoscopic cannula is advanced under endoscopic visualization, as previously described herein, either directly through the initial subxiphoid incision or through a cavity that is dilated using a dilation tool, as described herein. Upon reaching the pericardium, a flap of the pericardium is gripped using a pericardial entry instrument, as described herein, and the flap is cut to create an opening in the pericardium. Alternatively, the pericardial entry instrument may be aligned substantially tangentially to the pericardium under endoscopic visualization in gripping a flap of the pericardium. The flap of the pericardium is cut at a stretched spacing away from the underlying heart.

The subxiphoid approach method facilitates accessing all regions of the heart including the anterior, posterior, left and right regions of the heart. In one method embodiment, the cannula is initially inserted into the pericardium via an opening formed near the apex of the heart for access to anterior and posterior surfaces of the heart. Also, entry near the apex of the heart aids the surgeon by providing a landmark for easier recognition of the position of the endoscopic cannula within the body. Of course, other entry positions, such as entry in the posterior region of the heart, may also be selected. Once inside the pericardium, the cannula can be maneuvered around the heart substantially because of the subxiphoid entry and the flexibility of soft tissue around the heart. Thus, all regions of the heart may be accessed without the need for invasively lifting or rotating the heart to access posterior or lateral vessels and structures.

The subxiphoid access method is performed under endoscopic visualization and is minimally invasive. In addition, access through a subxiphoid incision obviates going through the pleural cavity and the associated deflation of a lung, and permits access to all regions of the heart via a single incision, without going through the pleural cavity.

In one embodiment of the present invention, the endoscopic cannula with the transparent tapered tip is used to bluntly dissect a path to the pericardium, through the fat and connective tissue. Direct visualization allows verification that the pericardial surface is clean and devoid of adherent fat. Application of the pericardial entry instrument may occur under visual guidance on an exposed pericardial surface.

In an alternative method embodiment of the present invention, after making the subxiphoid incision and inserting the endoscopic cannula in the incision, the endoscopic cannula is advanced to the mediastinum under endoscopic visualization for performing a surgical procedure on structures, other than the heart, that are located within the mediastinum, for example, the esophagus and the lymphatic glands. Thus, a biopsy specimen may be taken from a lymphatic gland using this procedure in accordance with the present invention.

In another embodiment of the present invention for accessing the heart within the pericardium, the heart is restrained by at least partially enclosing the heart with a cardiac restraint apparatus.

One embodiment of a cardiac restraint apparatus according to the present invention comprises a jacket having a rim which defines an opening for receiving a heart, and a strand that extends around the rim of the jacket and is tied into a slipknot. The apparatus also comprises a knot pusher that has a hollow elongate body with at least one end portion of the strand extending through the knot pusher for manipulating the slip knot by pulling the end portion of the strand away from the heart while pushing the knot pusher against the slipknot to reduce the diameter of the opening defined by the rim. In addition, the apparatus comprises one or more guide elements that are attached to the jacket.

In another embodiment of a cardiac restraint apparatus according to the present invention, the jacket is folded to reduce the profile of the apparatus. Optionally, the folded jacket is enclosed by a sheath. One embodiment of such a sheath includes a generally cylindrical body having a proximal end and a distal end, and also includes perforations along the sheath body to facilitate removal of the sheath from the apparatus by tearing the sheath body along the perforations. Optionally, a pull tab is attached to the proximal ends of the sheath body for removal by pulling the pull tab away from the jacket to tear the sheath long the perforations and remove the torn sheath from the patient.

In one embodiment of a cardiac restraint apparatus according to the present invention, the strand extending around the rim of the jacket is a suture strand, for example, formed of nylon. Also, the guide elements may include one or more hollow guide tubes that are removably attached to the rim of the jacket, and at least one of the guide tubes may define a lumen dimensioned to receive a surgical instrument, for example a tacking instrument. In other embodiments, the guide elements are handles, for example, including suture strands, attached to the rim of the jacket.

In other embodiments of the present invention, the apparatus comprises at least one elastic band having a first portion terminating at a first end and a second portion terminating at a second end, with the first portion and the second portion of the elastic band being joined together at a location between the first end and the second end.

The elastic band includes calibrated markings for calibrating the tension of the elastic band. In other embodiments, the first and second ends of the elastic band are configured to be engaged by a grasping instrument.

In one method embodiment of the present invention, a heart is at least partially enclosed with a cardiac restraint apparatus that includes a jacket. The method comprises the steps of: a) making a surgical incision to provide an entry point for the cardiac restraint apparatus; b) introducing a pericardium entry instrument through the incision and using the instrument to make an opening in the pericardium through which the cardiac restraint apparatus can be advanced into engagement with the heart; c) advancing the cardiac restraint apparatus through the incision and the opening into engagement with the heart; d) sweeping the jacket around the heart to at least partially enclose the heart in the jacket. The initial surgical incision can be a subxiphoid incision, a trans-xiphoid incision, a thorascopic incision, or other incision.

An alternative embodiment of the inventive method includes the steps of: a) making a surgical incision to provide an entry point for an endoscopic cannula; b) inserting into the surgical incision an endoscopic cannula that has at least one lumen or access port; c) advancing the endoscopic cannula to the pericardium under endoscopic visualization; d) introducing a peridcardium entry instrument into the access port of the endoscopic cannula; e) making an opening in the pericardium using the entry instrument through which the cardiac restraint apparatus can be advanced into engagement with the heart; f) advancing the endoscopic cannula through the pericardium through the opening; g) advancing the cardiac restraint apparatus through one lumen of the endoscopic cannula into engagement with the heart; h) sweeping the jacket around the heart to at least partially enclose the heart in the jacket.

Another embodiment of a method according to the invention uses the embodiment of the cardiac restraint apparatus that includes a jacket and one or more guide tubes. In this method, the step of enclosing the heart with the cardiac restraint apparatus includes the steps of: a) advancing a tacking instrument into at least one access port of the endoscopic cannula to access the pericardium; b) tacking the rim of the jacket to the posterior pericardium using the tacking instrument; and c) manipulating the guide tubes of the cardiac restraint apparatus to sweep the jacket over the anterior aspect of the heart thereby at least partially enclosing the heart with the jacket. The jacket is then tightened around the heart by reducing the diameter of the opening of the jacket by pulling the end portion of the strand away from the heart while pushing the knot pusher against the slipknot.

Another embodiment of a method according to the invention uses the embodiment of the cardiac restraint apparatus that includes a jacket and one or more handles. In this method, the step of enclosing the heart with the cardiac restraint apparatus includes the steps of: a) advancing one or more guide strands through at least one lumen of the endoscopic cannula, the one or more guide strands having sufficient length to enable the proximal ends of the one or more guide strands to be grasped outside the body as the distal ends of the guide strands are positioned near the endoscopic cannula; b) advancing a tacking instrument into one lumen of the endoscopic cannula; c) tacking the one or more guide strands to the posterior pericardium using the tacking instrument; d) passing the one or more guide strands through the one or more handles on the rim; and e) using the guide strands to manipulate the jacket to at least partially enclose the heart with the jacket.

Another embodiment of a method of restraining the heart involves a cardiac restraint apparatus that includes an elastic band. The method comprises the steps of: a) making a surgical incision to provide an entry point for the cardiac restraint apparatus; b) using a pericardial entry instrument introduced through the incision to make an opening in the pericardium through which the cardiac restraint apparatus can be advanced into engagement with the heart; c) advancing the cardiac restraint apparatus through the incision and the opening into engagement with the heart; and d) restraining the heart with the elastic band by securing the elastic band around the heart. This method includes forming the surgical incision as one of a subxiphoid incision, a transxiphoid incision, and a thorascopic incision.

An alternative embodiment of this method includes the steps of: a) making a surgical incision to provide an entry point for an endoscopic cannula; b) inserting into the surgical incision an endoscopic cannula that has at least one lumen or access port; c) advancing the endoscopic cannula to the pericardium under endoscopic visualization; d) using a pericardium entry instrument introduced through the access port of the cannula to make an opening in the pericardium through which the cardiac restraint apparatus can be advanced into engagement with the heart; e) advancing the endoscopic cannula into the pericardium through the opening; f) advancing the cardiac restraint apparatus through one lumen of the endoscopic cannula into engagement with the heart; and g) restraining the heart with the elastic band by securing the elastic band around the heart.

In the methods using the cardiac restraint apparatus having at least one elastic band, in one embodiment the step of restraining the heart with the cardiac restraint apparatus can include the steps of: a) advancing a tacking instrument into the opening in the pericardium (or, in the minimally invasive methods, into the lumen of the endoscopic cannula to access the pericardium; b) tacking the elastic band to the posterior pericardium at a point between the first end and the second end; c) grasping the first portion, moving the first portion to the anterior aspect of the heart; and tacking the first portion to the pericardium overlying the anterior aspect of the heart; d) grasping the second portion, moving the second portion over the anterior aspect of the heart, and tacking the second portion to the pericardium overlying the anterior aspect of the heart; and e) attaching (preferably by tacking or clipping) the first and second portions together (preferably at a location overlying the anterior aspect of the heart) to provide a calibrated tension on the heart. The steps of grasping and attaching together the first and second portions of the elastic band may be performed with any of a variety of tools, for example a clip applier.

In accordance with another embodiment of the present invention, an endoscopic cannula is used to enter the pericardium from the subxiphoid approach to attach epicardial tacks and to tension the epicardium between tacks around the annulus of the mitral valve. Specifically, two or more tacks are placed on the epicardial surface near the mitral annulus. The tacks are connected by a suture or wire that may be tensioned to alter the shape and size of the annulus. The tacks may be placed immediately inferior to the left circumflex artery, in the area corresponding to the anterior aspect of the mitral annulus, and immediately inferior to the coronary sinus, in the area corresponding to the posterior aspect of the mitral annulus. The tacks may be helical or spiral titanium tacks of a type, for example, similar to tacks used to fixate prosthetic mesh in laparoscopic hernia repair. Two or more tacks may be inserted into the myocardium, and a suture or wire strand may be threaded through the portion of the tacks that is not embedded into the myocardium. The suture or wire contains loops spaced at varying distances for looping onto the tacks to adjust the amount of tension between the tacks. Tensioning the epicardium in this manner decreases the size of the mitral annulus and corrects the regurgitation due to annular dilation.

In accordance with another embodiment of the present invention, a reinforcement device is placed over the heart using an endoscopic technique through a small incision. The pericardial sac encloses the heart and is not generally distensible in the short term, although it does increase in size over the long term with cardiomegaly in congestive heart failure. An endoscopic procedure in accordance with the present invention alters the pericardial sac to allow it to expand to a predetermined amount and then prevent further distention.

In accordance with illustrated embodiments of the present invention, a substantially rigid cannula includes separate elongated lumens extending between distal and proximal ends of the cannula to provide an instrument channel and one or more separate vacuum channels that terminate in a suction port located adjacent the distal end of the cannula. The instrument channel is sized to accommodate various surgical instruments including a hollow needle for penetrating the myocardium, for example, to deliver cells. The needle is configured for shallow penetration to avoid puncturing into a chamber of the heart with associated complications. In an alternative embodiment, an instrument carries a ‘needle’ that is sized to accommodate epicardial pacing or defibrillating leads within a closed channel that can be reconfigured into an open channel for releasing the leads. Additionally, the cannula with separate lumens or channels therethrough may be incorporated with or disposed within an instrument channel of an endoscopic cannula that houses an endoscope aligned with a distal transparent tip. This assemblage of surgical instruments may be conveniently positioned through tissue disposed between a subxiphoid incision and a surgical site on the pericardium of a beating heart, or positioned through tissue disposed between a thoracotomy incision and a surgical site on the pericardium of a beating heart (or through an opening in the pericardium and a surgical site on the myocardium). For some surgical procedures, a laterally expandable sheath may be employed to form a working cavity in tissue to facilitate the placement of the vacuum port and associated instrument channel at the surgical site on the pericardium (or myocardium).

In an embodiment of the present invention, a guide tube carries a suction tube slidably therein and supports a lead-placing channel thereon which includes rotatable or slidable half sections that house a cardiac pacing or defibrillating lead. The lead-placing channel can be configured to enclose a cardiac lead and to release the lead along a longitudinal slot therein that results from reconfiguring the channel after placement of a distal end of the cardiac lead into the myocardium. The suction tube terminates as its distal end in a suction pod that can provide temporary suction attachment of the assembly at a selected surgical location, for example, on the myocardium of a beating heart while a cardiac lead is manipulated within the placement channel to anchor the distal end of the cardiac lead to the myocardium.

In accordance with another embodiment of the present invention an endoscopic cannula is used to enter the pericardium from a subxiphoid approach, visualize the superior vena cava, and place an illuminated clip on the pericardium adjacent the superior vena cava. The clip contains an attached light emitting diode (LED) that is mounted to emit light from the tip of the clip. The endoscopic subxiphoid cannula is used to visualize the inferior vena cava, and a light emitting clip is attached to the pericardium adjacent the inferior vena cava. In another embodiment, an elongated light ‘stick’ or a light-emitting endoscope can have a distal end positioned adjacent the inferior vena cava, and a second endoscope can be guided toward the position of the first source of light. The subxiphoid endoscopic cannula is then removed from the mediastinum and inserted into the right pleural cavity through a small thoracotomy incision. The transilluminating light from each clip guides the tissue-dissecting cannula during dissection under the superior and inferior vena cava, respectively. Dissection is performed via a combination of blunt dissection with a transparent tapered tip of the cannula, and dissection with the pericardial entry instrument.

Following dissection posterior to the inferior vena cava and dissection posterior to the superior vena cava, a flexible elongated probe or a flexible tubular sheath is used to encircle the pulmonary veins. The probe or sheath starts in the right pleural cavity, tracks posterior to the superior vena cava, then tracks along the transverse sinus superior to the right and left superior pulmonary veins, then inferior to the left and right inferior pulmonary veins, and posterior to the inferior vena cava, back out into the right pleural cavity. An ablation probe is advanced along the dissected path and energy is applied to ablate atrial tissue surrounding the pulmonary veins.

In accordance with another embodiment of the present invention, two probes may be advanced along the posterior pericardial surface around different courses to substantially encircle the four pulmonary veins, with the tips of the probes separated by a reflection (i.e., a partition, as used herein, formed of dense tissue) of the pericardium along the back of the superior vena cava, and by a pericardial reflection between the right inferior pulmonary vein and the inferior vena cava. The two probes nearly touch each other, separated by the pericardial reflections, in substantial encirclement of the pulmonary veins, and magnetic tips and bands are disposed on the probes to aid in aligning the probes on the opposite sides of the pericardial reflections. An ablation probe is laterally flexible and torsionally rigid to assure proper orientation of applied tissue-ablating energy relative to cardiac tissue along the encircling path around the pulmonary veins. In another procedure according to the present invention, a single endoscopic cannula is used to position an ablation probe around the right and left pulmonary veins via right inter-costal thoracotomy and subxiphoid incisions. In still another procedure according to the present invention, a vacuum-assisted cannula is advanced through the endoscopic subxiphoid cannula for temporary vacuum-controlled attachment to the epicardial surface of the heart.

In another embodiment of the present invention, ablation of atrial tissue surrounding the four pulmonary veins may be accomplished using a combined intrapericardial and extrapericardial technique. First, a subxiphoid incision is used to gain access to and enter the pericardium. An ablation probe is advanced into the transverse pericardial sinus to its termination near the right superior pulmonary vein. The probe tip lies at the end of the transverse sinus, while its body encircles the four pulmonary veins on three sides, i.e., (1) superior to the superior pulmonary veins, (2) lateral to the left superior and left inferior pulmonary veins, and (3) inferior to the inferior pulmonary veins. This leaves completing the one side that is lateral to the right superior and right inferior pulmonary veins.

Dissection of tissue lateral to the right superior and right inferior pulmonary veins is hazardous due to the presence of the vena cava. Puncture or laceration of this large diameter, thin walled vessel during a closed-chest, endoscopic procedure is dangerous because of limited access to control hemorrhage. An extrapericardial approach avoids dissection of the vena cava and utilizes a tissue plane directly posterior and lateral to the right superior and right inferior pulmonary veins. Tissue-ablating energy can be applied through the posterior pericardium, onto the atrial tissue lateral to the right superior and inferior pulmonary veins. The endoscopic subxiphoid cannula facilitates dissecting an extrapericardial plane lateral to the right pulmonary veins. The right inferior pulmonary vein is visualized by the endoscopic subxiphoid cannula, and the pericardial entry instrument is used to grasp the posterior pericardium lateral to the right inferior pulmonary vein. A small opening is formed by the pericardial entry instrument, and the endoscopic subxiphoid cannula is advanced through this opening in a superior direction, until an extrapericardial tract is formed lateral to the right pulmonary veins, extending from below the right inferior pulmonary vein to above the right superior pulmonary vein. An ablation probe may be advanced into this tract and oriented toward the atrial tissue lateral to the right pulmonary veins.

Dissection of the extrapericardial tract using the endoscopic subxiphoid cannula may be facilitated by prior placement of a lighted indicator at the end of the transverse pericardial sinus. The light transilluminates through the posterior pericardium to provide an indicator guiding the advancement of the endoscopic subxiphoid cannula as it dissects from the right inferior pulmonary vein to the right superior pulmonary vein.

### BRIEF DESCRIPTION OF THE DRAWINGS

FIG. 1A is a perspective view illustrating a dilation tool in accordance with the present invention.

FIG. 1B is a perspective view illustrating the inner cannula of the dilation tool of FIG. 1A.

FIG. 1C is a perspective view illustrating the expandable sheath of the dilation tool of FIG. 1A.

FIG. 1D is a cross sectional view of the inner cannula of the dilation tool of FIG. 1B.

FIG. 1E is a perspective view illustrating an embodiment of the slide mount of the dilation tool of FIG. 1A.

FIG. 1F is a perspective view illustrating an embodiment of the housing of the dilation tool of FIG. 1A.

FIG. 2 is a flow chart illustrating a method of using the dilation tool in accordance with the present invention.

FIGS. 3A-D are perspective views illustrating the dilation tool in operation in accordance with the present invention.

FIG. 4 is a perspective view illustrating a pericardial entry instrument in accordance with the present invention.

FIG. 5 is a flowchart illustrating a method of using the pericardial entry instrument of FIG. 4.

FIGS. 6A-D are perspective views illustrating operation of the pericardial entry instrument in accordance with the present invention.

FIG. 7A is a perspective view of an endoscopic cannula with a lumen or access port in accordance with the present invention.

FIG. 7B is a perspective view of an endoscopic cannula having an access port and an articulable head in accordance with the present invention.

FIG. 7C is a cross sectional view of the embodiment of FIG. 7B.

FIG. 7D is a perspective view of an endoscopic cannula in accordance with the present invention that is substantially arcuate in shape.

FIG. 8A is a flowchart illustrating the subxiphoid access method of using an endoscopic cannula via a tissue cavity that is dilated using the dilation tool with an expandable sheath in accordance with the present invention, as well as an alternative method of using the endoscopic cannula and pericardial entry instrument in accordance with the present invention, without first dilating a cavity, for procedures performed within the mediastinum.

FIG. 8B is a flowchart illustrating two alternative methods of using an endoscopic cannula and pericardial entry instrument of the present invention, for procedures performed within the pericardium.

FIGS. 9A-D are partial cross sectional views illustrating the operation of an endoscopic cannula and dilation tool in accordance with the present invention.

FIGS. 10A-E are partial cross sectional views illustrating the operation of an endoscopic cannula, dilation tool and pericardial entry instrument in accordance with the present invention.

FIGS. 11A-C are partial cross sectional views illustrating 360° access to the heart using the subxiphoid access method of the present invention.

FIG. 12A is a perspective view of a longitudinal mechanical dilator in accordance with another embodiment of the present invention.

FIG. 12B is a perspective view of the dilator of FIG. 12a in which the inner cannula is partially withdrawn through an expandable sheath in accordance with the present invention.

FIG. 12C is a perspective view of the dilator of FIG. 12b in which the inner cannula is further withdrawn through the expandable sheath in accordance with the present invention.

FIG. 13 is a flow chart illustrating a method of dilating tissue in accordance with the present invention.

FIG. 14 is a perspective exploded view illustrating an alternate embodiment of the longitudinal mechanical dilator in which the expandable sheath is removable from the inner cannula.

FIGS. 15A-D are perspective views of an embodiment of a split tissue-expansion device in accordance with the present invention.

FIG. 16 is a perspective view of one embodiment of a cardiac restraint apparatus of the present invention.

FIG. 17 is a partial cross sectional view of the operation of the knot pusher in reducing the diameter of the opening of an embodiment of a cardiac restraint apparatus according to the present invention.

FIG. 18 is a partial sectional view of the attachment of guide tubes to the rims of the cardiac restraint apparatus of FIG. 16.

FIG. 19 is a perspective view of an alternative embodiment of a cardiac restraint apparatus of the present invention.

FIG. 20 is a perspective view of a sheathed cardiac restraint apparatus of the present invention.

FIGS. 21A through 21G are partial cross sectional views of a method according to the present invention for accessing the heart with an endoscopic cannula using a subxiphoid approach.

FIGS. 22A through 22D are partial cross sectional views of the operation of an endoscopic cannula and the use of a cardiac restraint apparatus in accordance with the present invention.

FIGS. 23A through 23C are partial cross sectional views of an alternative method of the operation of an endoscopic cannula and the use of an alternative embodiment of a cardiac restraint apparatus in accordance with the present invention.

FIGS. 24A through 24B are perspective views of an alternative embodiment of a cardiac restraint apparatus according to the present invention.

FIGS. 25A through 25C are partial cross sectional views of the operation of an endoscopic cannula and the use of an alternative embodiment of a cardiac restraint apparatus according to the present invention.

FIG. 26 is a side view of a vacuum-assisted injection cannula in accordance with one embodiment of the present invention.

FIG. 27 is a side view of an endoscopic cannula for use with the methods of the present invention.

FIG. 28 is a partial side view of the assembled cannulas of FIGS. 26 and 27 in a surgical procedure according to the present invention.

FIG. 29 is a perspective view of another embodiment of a vacuum cannula in accordance with the present invention.

FIG. 30 is a plan view of a releasable guide for a cardiac lead according to another embodiment of the present invention.

FIG. 31 is a partial plan view of the distal end of the releasable guide in the embodiment of FIG. 30.

FIG. 32 is a partial plan view of the proximal end of the releasable guide in the embodiment of FIG. 30.

FIG. 33 is a top view of the distal end of the releasable guide in the embodiment of FIG. 30.

FIG. 34 is a perspective view of the distal end of the releasable guide according to the embodiment illustrated in FIG. 30.

FIG. 35 is a partial plan view of a releasable guide in accordance with the embodiment illustrated in FIG. 30.

FIG. 36 is a partial plan view of the releasable guide of FIG. 30 assembled within an endoscopic instrument in accordance with the present invention.

FIG. 37 is a pictorial illustration of the interior of the pericardial sac (anterior view, heart removed).

FIGS. 38A-D are, respectively, partial plan, end and sectional views of an endoscopic probe in accordance with one embodiment of the present invention.

FIG. 39 is a pictorial illustration of the path of an ablation cannula or probe prepared within the intrapericardial space in the illustration of FIG. 37 in accordance with the present invention.

FIGS. 40A through C are, respectively, side, bottom and end views of an ablation probe in accordance with one embodiment of the present invention.

FIG. 41 is a plan view of an ablation cannula or probe in accordance with another embodiment of the present invention.

FIG. 42 is a pictorial illustration of the path of ablation cannulas or probes within the intrapericardial space in the illustration of FIG. 37 achieved with probes of the embodiment illustrated in FIG. 41.

FIGS. 43A and 43B comprise a flow chart illustrating one surgical procedure according to the present invention.

FIGS. 44A and 44B comprise a flow chart illustrating another surgical procedure according to the present invention.

FIG. 45 is a pictorial illustration of an ablation probe and sheath according to one embodiment of the present invention.

FIG. 46 is a pictorial illustration of a configuration of the probe according to FIG. 45 following a surgical procedure according to the present invention.

FIGS. 47A and 47B comprise a flow chart illustrating a surgical procedure according to one embodiment of the present invention.

FIG. 48 is a top anatomical sectional view illustrating a surgical procedure according to the present invention.

FIG. 49 is a partial anatomical illustration of a surgical procedure according to the present invention.

FIG. 50 is a plan view of a suction cannula in accordance with one embodiment of the present invention.

FIGS. 51A and 51B are, respectively, bottom and top views of the suction pod of FIG. 50.

FIG. 52 is a plan view of a composite structure including a vacuum-assisted cannula slidably disposed within the endoscopic cannula in accordance with the present invention.

FIG. 53 is a pictorial view of a braided sheath that promotes torsional rigidity for properly orienting an ablation probe in accordance with the present invention.

FIG. 54 is an anterior view of the pericardial sac (without the heart) showing the path of an ablation probe in accordance with the present invention.

FIG. 55 is a partial top view of the heart showing the locations of epicardial tacks placed according to one embodiment of the surgical procedures of the present invention.

FIG. 56 is a partial anterior view of the heart showing the placement in the epicardium of the anterior tack in accordance with the present invention.

FIGS. 57A and 57B are pictorial illustrations of a knotted suture and apparatus for positioning and tensioning the suture between epicardial tacks in accordance with the present invention.

FIG. 58 is a plan view of the apparatus of FIG. 57B for installing the suture of FIG. 57A between epicardial tacks.

FIG. 59 is a partial top view of the heart showing the position of the suture loop between epicardial tacks in accordance with the present invention.

FIGS. 60A and 60B comprise a flow chart illustrating an embodiment of the surgical procedure in accordance with the present invention.

FIG. 61 is a pictorial illustration of an endoscopic cannula accessing the heart via the subxiphoid entry.

FIG. 62A is an end view of an instrument in accordance with the present invention for attaching tacks and bands to the pericardium.

FIG. 62B is a top view of the instrument of FIG. 62A including a plurality of tacks and attached bands traversing a yoke-like structure.

FIG. 62C is an end view of a tack in FIGS. 62A and 62B.

FIGS. 63A-C are side views of the operation of the instrument of FIG. 62A during installation of tacks and bands on the pericardium.

FIGS. 64A and 64B are plan views, respectively, of the instrument of FIG. 61A installing tack and bands, and of the installed tacks and bands on the pericardium.

FIG. 64C is a plan view of the procedure for cutting the pericardium between installed tacks.

FIG. 64D is a plan view of the heart illustrating the tacks and bands installed across opening formed in the pericardium.

FIGS. 65A and 65B comprise a flow chart illustrating the surgical procedure for ablating tissue along intrapericardial and extrapericardial tracks.

### DETAILED DESCRIPTION OF THE INVENTION

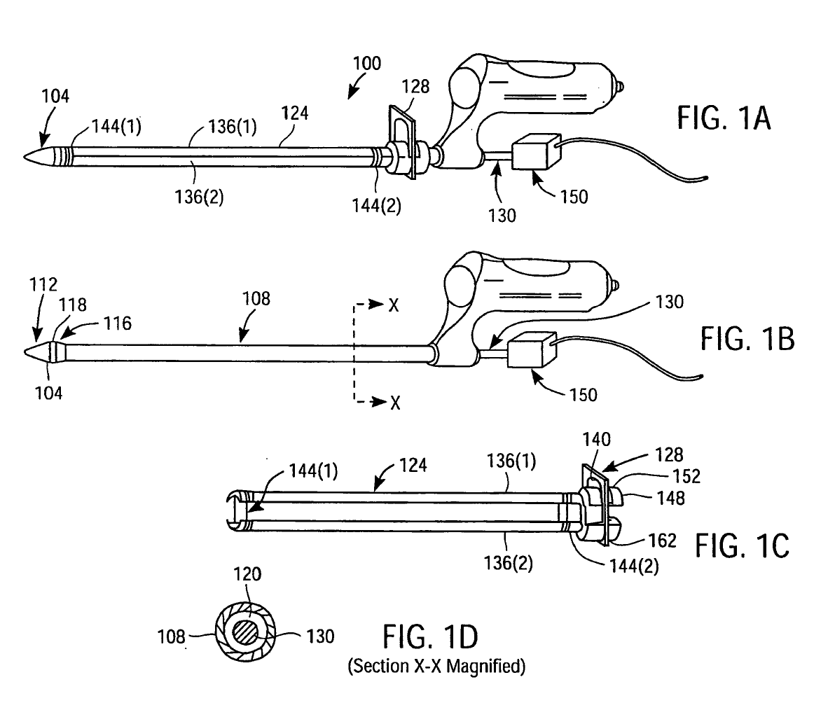

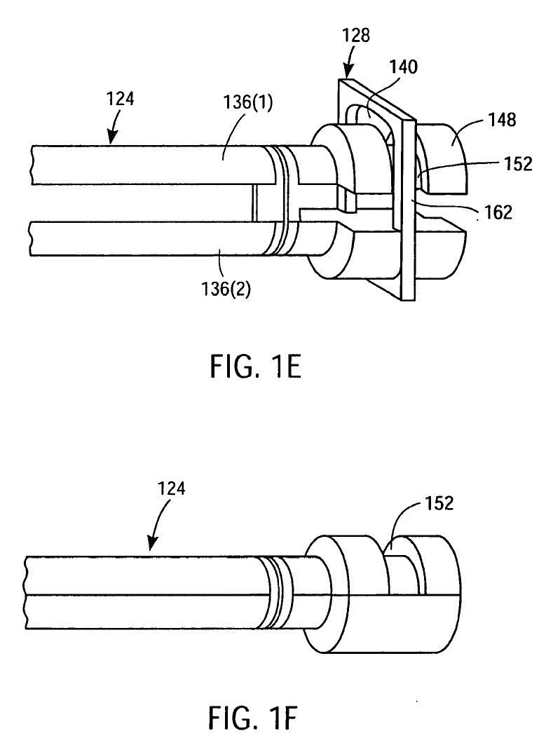

FIGS. 1A-D illustrate a preferred embodiment of a dilation tool 100 which embodies an aspect of the invention. Dilation tool 100 includes an inner cannula 108 having lumen 120 as shown in FIG. 1D, and an expandable sheath 124 comprised of shells 136(1) and 136(2) as shown in FIG. 1C. Preferably, the inner cannula is formed of a sufficiently rigid material, such as metal or plastic, that would allow tip 104 to be used to bluntly dissect a cavity from an incision point to the pericardium or other surgical site of interest. Lumen 120 is provided to allow the insertion of an endoscope 130 fitted with video camera 150 in the dilation tool 100, and tip 104 is transparent to allow endoscopic visualization during the surgical procedure. In a preferred embodiment, tip 104 has a long distal taper 112 as shown in FIG. 1B, which allows tip 104 to bluntly dissect away tissue encountered along the cavity to the pericardium. Conically-tapered tip 104 also provides a less distorted field of view than conventional tips. Tip 104 in the preferred embodiment also has a proximal short taper 116. The proximal short taper 116 facilitates the retraction of the inner cannula 108 through expandable sheath 124. Intermediate between proximal short taper 116 and long distal taper 112 is an optional enlarged region 118. The enlarged region 118 has a maximal dimension greater than the diameter of the inner rigid cannula 108, and this greater maximal dimension causes the expandable sheath 124 to expand as tip 104 is retracted through sheath 124. Tapered tip 104 is preferably configured to be removable from the elongate body, for example by means of being screwed into a threaded end of the elongated body, or by snapping to fit onto the elongated body.

Inner cannula 108 preferably has a relatively small diameter, for example 7 mm, which minimizes the probing force exerted on the heart caused by advancement of the dilation tool 100 to the anterior surface of the pericardium. The use of larger cannulas to isolate the anterior surface of the pericardium has a greater tendency to cause cardiac arrhythmias. However, in order to introduce pericardial puncture or entry instruments to the surgical site, an endoscopic cannula with an instrument lumen or access port must be advanced to the pericardium, and these cannulas typically have larger diameters, for example, 12 mm in diameter. Therefore, a cavity is preferably initially dilated to accommodate these larger cannulas.

In use of tool 100, as shown in FIG. 1A, expandable sheath 124 resides on the outside of inner cannula 108. Expandable sheath 124 allows insertion into the body of instruments of a diameter greater than the initial puncture size. In a preferred embodiment, as shown in FIG. 1C, the expandable sheath 124 is generally rigid and is split longitudinally into two shells 136(1) and 136(2). These shells of the expandable sheath 124 may be metal, plastic, or the like. Metal expandable sheaths may provide better dilation than plastic due to their superior rigidity.

As used in this application, the word “distal” describes that portion of the apparatus (or that direction of movement) which extends away from the user during use, and the word “proximal” describes that portion of the apparatus (or that direction of movement) that extends toward the user during use.

Expandable sheath 124 has a first resilient connector 144(1) near the proximal part of the sheath 124 and a second resilient connector 144(2) near the distal end of the sheath 124. The resilient connectors 144 are preferably elastic bands and contract the two shells 136(1) and (2) against inner cannula 108. The resiliency of connectors 144 allows expandable sheath 124 to expand along the longitudinal split as an object of greater diameter is advanced or withdrawn through sheath 124. In one embodiment, the inner surface of the distal end of the expandable sheath 124 is chamfered to facilitate easier withdrawal or retraction of the tip 104 through the expandable sheath 124. The proximal end of the expandable sheath 124 is attached to slide mount 128 which retains shells 136(1) and (2) of expandable sheath 124 in axial alignment as sheath 124 expands. Slide mount 128 may be formed of a hard plastic or other rigid material having a slot 140 disposed to fit in tracts or grooves in the proximal ends of the expandable sheath 124.

The lower shell 136(2) of the expandable sheath 124, is attached to the slide mount 128 in the embodiment illustrated in FIG. 1C. The upper shell 136(1) in FIG. 1C, is unattached, and is constrained to slide freely in a vertical direction within the slot 140. In one embodiment, axial alignment is maintained due to use of a housing 148. In this embodiment, shown in FIG. 1E, the unattached shell 136(1) has a housing 148 disposed at its proximal end. As shown in FIGS. 1E and 1F, housing 148 has a horizontal dimension greater than the horizontal dimension of the slot 140. However, housing 148 has a groove 152 which receives frame 162 of the slide mount 128 to facilitate slidably moving the housing 148 within groove 152 in the vertical direction. Groove 152 has a sufficiently narrow width to ensure minimal axial movement of shell 136(1) relative to frame 162. Thus, during advancement or retraction of a device, the unattached shell 136(1) is displaced vertically, but its axial movement is restricted.

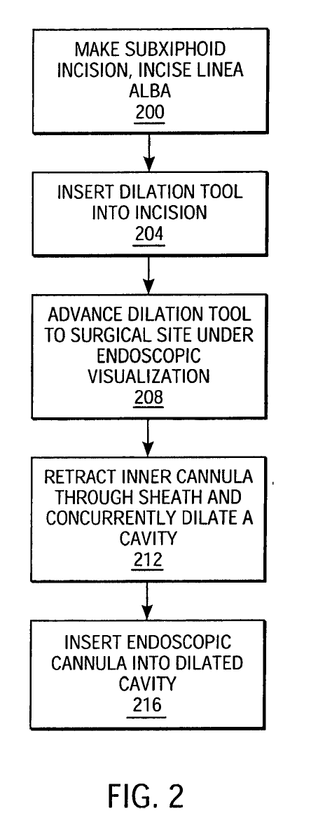

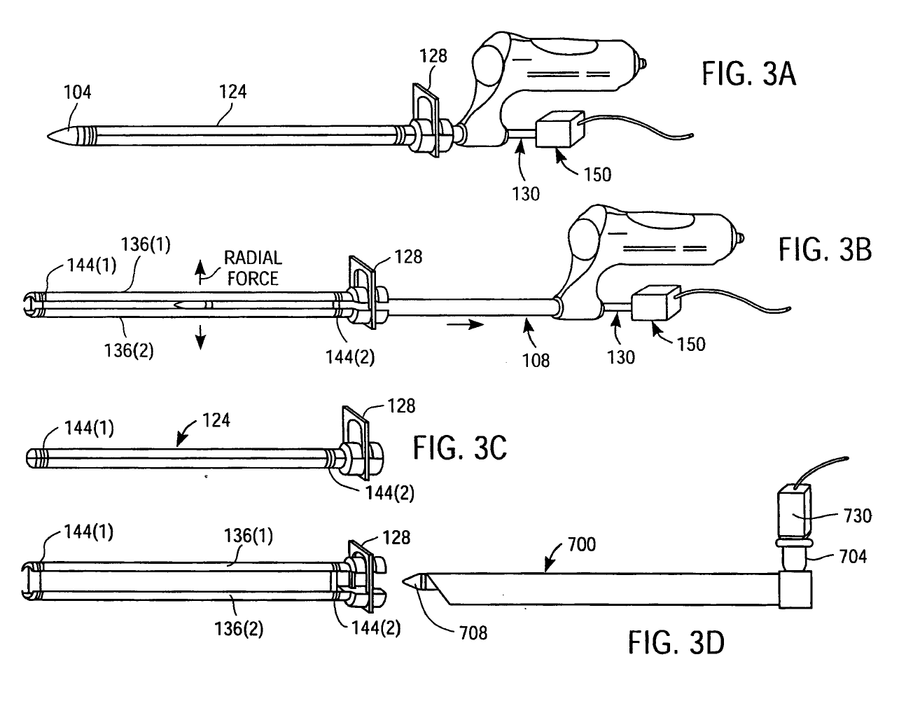

FIG. 2 is a flowchart which illustrates a method of using dilation tool 100, and will be described with reference to FIGS. 3A-3D, showing only the apparatus. In step 200, a subxiphoid incision is made overlying an entry point for a surgical procedure. An initial skin incision for a cardiac procedure may be performed either in the subxiphoid region, or in the intercostal space. The initial skin incision for an endoscopic vessel harvesting procedure may be near the groin, near the knee, or near the ankle.

A subxiphoid incision is preferably small, about 2 cm. Next, the subcutaneous tissue below the incision is bluntly dissected to expose the linea alba, which is also incised. Dilation tool 100 is inserted 204 into the incision, and tapered tip 104 bluntly dissects a cavity responsive to the advancement of the dilation tool 100. For an initial incision made in the subxiphoid region, dilation tool 100 is then positioned on the posterior aspect of the xiphoid process and sternum and may be used to sweep fat from the anterior surface of the pericardium. The dilation tool 100 is advanced 208 within the mediastinum (optionally to the pericardium) under endoscopic visualization. An endoscope with an attached CCD chip camera can be used to accomplish endoscopic visualization. Since the pericardium is a thin membrane, visualization of the beating heart through the endoscope underneath a translucent membrane indicates correct positioning of the dilation tool 100 on the anterior surface of the pericardium.

Following advancement of the dilation tool 100 to the desired position in the body, expandable sheath 124 is held in place as inner cannula 108 is retracted 212 through expandable sheath 124, as shown in FIG. 3B. Retraction of inner cannula 108 with enlarged region 118 through the length of expandable sheath 124 dilates the tissue adjacent to the length of expandable sheath 128 to at least the maximal dimension of the enlarged region 118. The slide mount 128 is held in place, while the inner rigid cannula 108 is withdrawn or removed. The proximal taper 116 of cannula tip 104 rides against the chamfered inner surface of the distal end of the expandable sheath 128, smoothing out the initial process of cannula removal.

The inner cannula tip 104 glides along the inner surfaces of the two shells 136 during cannula withdrawal. The generally rigid structure of the split shells radially displaces the surrounding tissue as the shells part or separate, thus dilating the cavity initially created by advancement 208 of dilation tool 100. Thus, substantially all of the force resulting from withdrawing cannula tip 108 is exerted on the inner surfaces of the shells 136, and not on the tissue and this advantageously isolated the shear force from causing vessel avulsion and tissue abrasion during tissue dilation. In accordance with the present invention, radial force is exerted on the tissue by the split shells 136 to reduce any trauma to the tissue from the dilation process. The dilation of the cavity facilitates subsequent insertion 216 into the lumens of larger diameter instruments, particularly the endoscopic cannula of the present invention.

In one embodiment, expandable sheath 124 remains in position within the patient's body (not shown) in the dilated cavity created by removing inner cannula 108 as shown in FIG. 3B. Large diameter instruments are sequentially inserted 216 through the proximal ends of expandable sheath 124, without exerting shear force on the tissue cavity. Expandable sheath 124 accommodates instruments of varying diameters and cross-sections. Additionally, leaving expandable sheath 124 in place maintains a dilated cavity to the desired surgical site, thus facilitating the advancement of the next instrument to be used in the procedure to the correct position within the body. FIG. 3D illustrates an endoscopic cannula 700 according to the present invention about to be inserted into expandable sheath 124, which is expanded as shown in FIG. 3d to accommodate the larger diameter of the endoscopic cannula.

Advancement of the larger cannula dilates the dissection cavity to the exact size necessary to accommodate the larger cannula. Therefore, in accordance with the present invention, the cavity is dilated no larger than required to accommodate the surgical tools used in the procedure. In the prior art, a surgeon would have to estimate the amount of dilation required for a procedure, and would have to repeatedly dilate the tunnel if the surgeon underestimated the amount of dilation required. Conversely, over-estimating the amount of dilation required leads to unnecessary trauma. This is avoided through the use of the expandable sheath 124 which expands concurrent with the size of the tool inserted.

In another embodiment, the expandable sheath 124 is slidably attached to the inner cannula 108. In this embodiment, the inner cannula 108 is retracted through the expandable sheath 124 as described above, but the expandable sheath remains positioned at the distal end of the dilation tool 100. After dilation has been achieved using the expandable sheath 124, the entire dilation tool 100 is removed from the body.

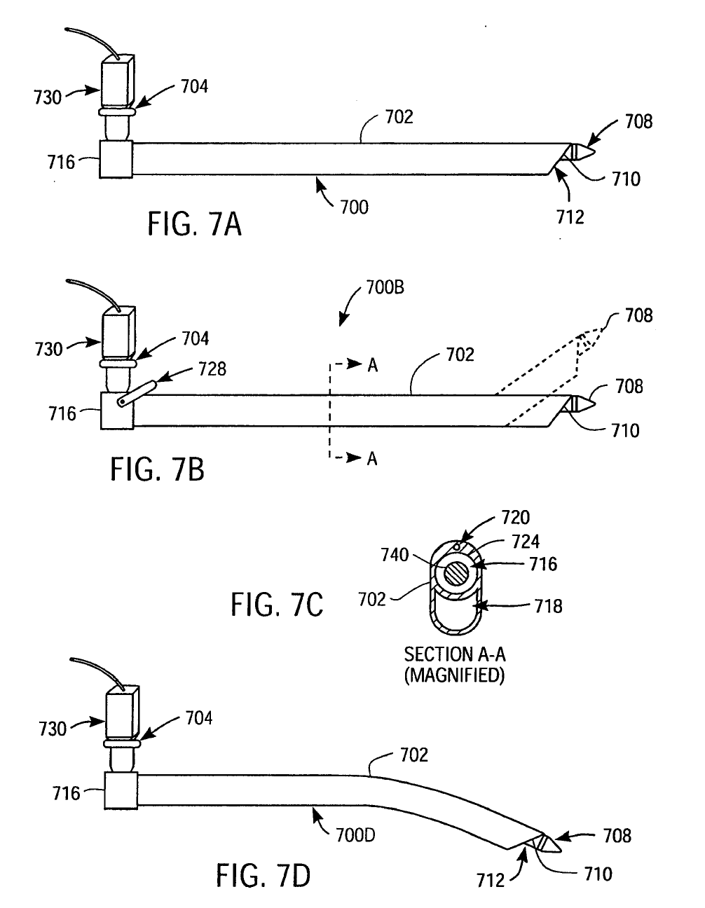

As previously mentioned, dilation tool 100 may be used with a larger diameter instrument for facilitating the insertion of the larger diameter instrument by dilating a cavity to the surgical site within the patient's body. One such larger diameter instrument is an endoscopic cannula according to the present invention. Referring now to FIGS. 7A-D, endoscopic cannula 700 comprises cannula 702 having an elongated body and defining one or more lumens 716 and 718. One of the lumens may be used as an endoscopic lumen 716 to house the endoscope 740, while the other lumen 718 is used as an access port for housing surgical devices, advanced either concurrently or sequentially, as will be discussed more specifically below. Endoscopic cannula 700 further comprises transparent tip 708 positioned at a distal end of cannula 702 in line with an endoscope 740 for visualization of the surgical procedure. Tip 708 is preferably tapered, and most preferably cone shaped, as shown in FIG. 7A. Cannula 702 may be constructed in any suitable configuration, for example, as a rigid body containing lumens 718 and 716. Alternatively, cannula 702 may contain a smaller diameter dissection shaft 710 defining lumen 716, the shaft 710 terminating in tip 708 at its proximal end.

In one embodiment, endoscope 740 is used with an eyepiece 704 skewed at a right or oblique angle to endoscope 740 to allow eyepiece 704 to be positioned away from the plane in which access port 718 resides. This arrangement prevents interference between a video camera 730 (attached to the eyepiece 704 of the endoscope) and a handle of a pericardial entry instrument (not shown). FIG. 7A illustrates endoscopic cannula 700 housing an eyepiece 704 at a right angle to endoscope 740. By positioning eyepiece 704 at a right angle to endoscope 740, rigid instruments may be inserted through access port 718 without interfering with camera 730. Alternatively, eyepiece 704 may be oriented along the longitudinal axis of endoscope 740. If eyepiece 704 is oriented in this alternative position, flexible instruments are inserted through access port 718 to avoid interfering with camera 730. The tapered profiles of these devices may facilitate subxiphoid dissection to the pericardial surface in sufficiently atraumatic manner to avoid the need for using dilation tool with an expandable sheath (shown in FIG. 1A) prior to advancement of the endoscopic cannula with an access port (shown in FIG. 7A).

The endoscope 740 is approximately 4-5 mm in diameter, and the access port 718 is approximately 7 mm in diameter. Access port 718 is sufficiently wide to permit the introduction of the necessary surgical instruments to perform the operation. Endoscope 740 in the endoscopic cannula 700 is sealed inside a transparent tapered tip 708 to preserve visualization as the endoscopic cannula 700 contacts tissue or fluids such as blood or pericardial fluid.

The endoscopic cannula 700 may be substantially straight as shown in FIG. 7A and is constructed of a rigid material such as metal or resilient plastic to permit creation of a cavity by blunt dissection resulting from advancement of the cannula within the body. Endoscopic cannula 700 may have any suitable profile, for example elliptical (as shown in FIG. 7C) or circular. In an alternative embodiment as shown in FIG. 7D, the endoscopic cannula 700D is rigid but substantially arcuate in shape. In another alternative embodiment, illustrated by articulating cannula 700B in FIG. 7B, the endoscopic cannula is constructed of a flexible material, such as flexible plastic (polyethylene, polyurethane, polytetrafluroroethylene, or the like) and its tip 708 is articulable, for example, with the aid of a wire 720 running through a separate wire lumen 724 to the distal end of the device, as shown in FIGS. 7B and 7C. Tensioning the wire 720 at its proximal end causes the cannula tip 708 to bend. Use of a flexible fiberoptic endoscope and a flexible endoscopic instrument in an articulating cannula 700B enables access into tight regions.

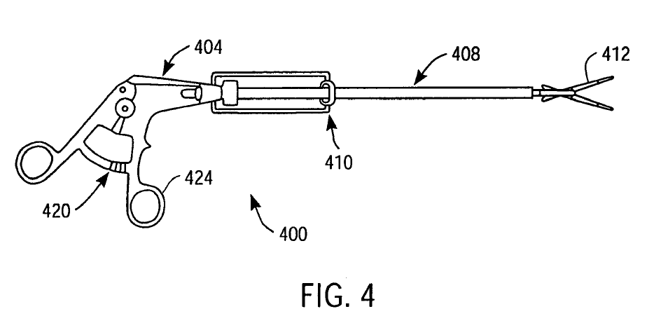

As previously discussed, endoscopic cannula 700 is used in conjunction with surgical instruments which are inserted either concurrently or sequentially into an access port or lumen of the endoscopic cannula. One such surgical instrument is the pericardial entry instrument of the present invention. FIG. 4 illustrates a perspective view of one embodiment of pericardial entry instrument 400. The instrument 400 includes a grasping tool 404 and a cutting tool 408. The grasping tool 404 includes a pair of locking endoscopic grasping forceps or jaws 412 of, for example, approximately 5 mm diameter, as smaller diameter forceps may not provide sufficient force to dissect fatty tissue adherent to the pericardium, and to grasp the pericardium during cutting. Upon access to the pericardium, the grasping jaws 412 of the grasping tool 404 pinch together pericardial tissue to create a flap of pericardium. The cutting tool 408 is then extended out over the forceps to cut the gripped flap of pericardium, creating a small opening within which other surgical instruments may be introduced. The cutting tool 408 is a tubular cutter that has a sharpened distal edge and that is positioned concentrically about a shaft of the grasping tool 404. The tubular cutter 408 is disposed to facilitate free rotation about the shaft of the grasping tool 404 to facilitate the cutting of the pericardial tissue. The tubular cutter 408 is also slidably disposed on the shaft of the grasping tool 404 to facilitate axial translation from an initial position proximal to the grasping jaws 412 of the grasping tool 404 to a final position a short distance distal to the distal end of the grasping jaws 412 sufficient for cutting the pericardium.

In one embodiment, an extension limiter 410 is disposed near the proximal end of the instrument 400 to restrict the range of axial translation of the cutting tool 408. The extension limiter 410 allows the surgeon to push the cutting tool 408 forward without fear of accidentally advancing the cutting tool 408 through the pericardium, into the underlying heart. The cutting tool 408 cuts a small (approximately 5 mm diameter) hole in the pericardium responsive to being advanced into the gripped flap and being rotated upon contact. The procedure is performed under direct endoscopic visualization to avoid injury to the heart which lies in contact with the inner surface of the pericardium.

The pericardial entry instrument 400 also includes a ratchet lock 420 disposed as part of the scissor handle 424. When scissor handle 424 is closed, the grasping tool jaws 412 are closed. The ratchet lock 420 locks the jaws 412 into their closed position when the scissor handle 424 is closed. This allows the flap of the pericardium to be held securely while the cutting tool 408 is advanced into the pericardium.

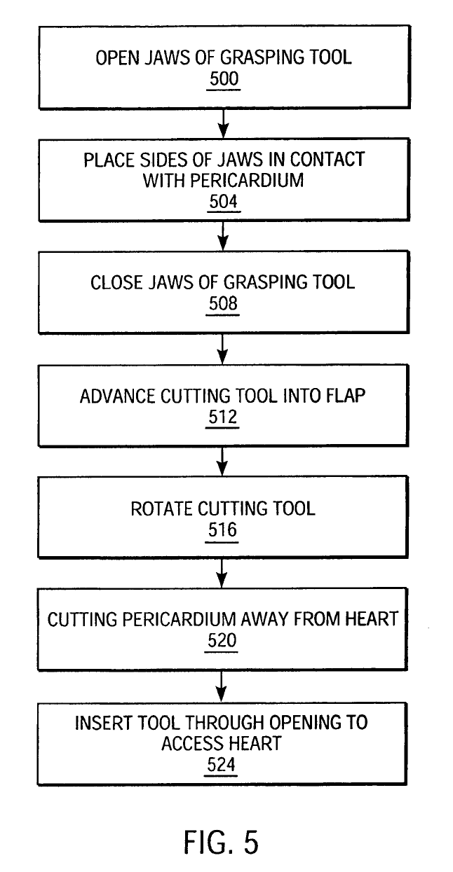

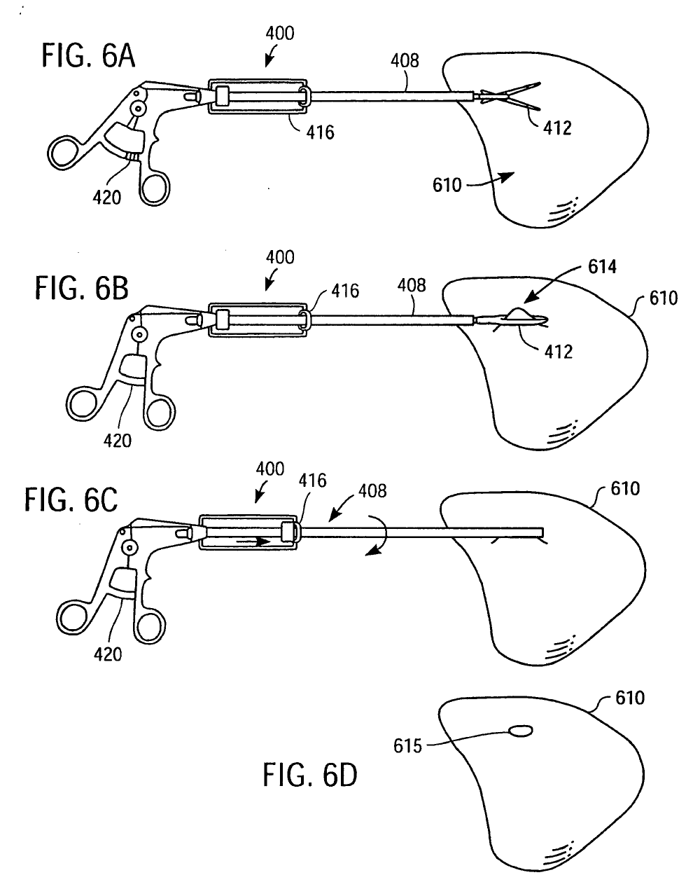

FIG. 5 is a flowchart which illustrates a method of using the pericardial entry instrument 400, as described with reference to FIGS. 6A-6D. In use, the jaws 412 of the grasping tool 404 are opened 500, and the sides of the open jaws 412 are placed in contact 504 with the pericardium 610, as shown in FIG. 6A. Jaws 412 are closed 508 to tent up a fold 614 of pericardium 610 as shown in FIG. 6B, while the underlying epicardial surface slips away from the grasp of the jaws 412, thereby preventing pinching of the heart. Ratchet lock 424 is activated when the grasping tool jaws 412 is closed to hold the pericardial fold 614 securely. Cutting tool 408 is advanced 512 toward the fold and is rotated simultaneously 516 to cut an opening 615 in the tented fold 614 of the pericardium, as shown in FIG. 6C. The pericardium 610 is grasped along the side of the grasping tool jaws 412, to facilitate tangential movement of the cutting tool 408 with respect to the surface of the heart. Therefore, the tented fold 614 of pericardium is cut 520 in a direction away from the underlying heart to avoid injury to the heart.

In the pericardial entry instrument 400, application of the forceps jaws in a tangential relationship to the surface of the heart at the site of pericardial entry ensures that no injury occurs to the heart. The cutting tool is in intimate contact with the forceps jaws. As it slices through the flap of pericardium held in the jaws, the cutting tube also lies tangential to the surface of the heart, and the surface of the heart is moved away without being cut. In contrast, if the pericardium were to be grasped by the distal tips of the forceps jaws in substantially normal alignment with the pericardium at the target site, then advancement of the cutting tool would occur in a direction perpendicular to the surface of the heart and entry into the heart muscle with attendant injury would be much more likely.

As shown in FIG. 6D, a small opening 615 with a cleanly cut edge is thus formed in the pericardium 610. Using endoscopic cannula 700 as previously described, surgical tools may be inserted 524 via an access port of the endoscopic cannula through the opening 615 to access the heart and perform the desired therapeutic procedure. The desired surgical and therapeutic procedures which can be performed at this point include but are not limited to such procedures as epicardial mapping and ablation for atrial and ventricular arrhythmias, pericardial window, myocardial biopsy, intrapericardial drug delivery, inserting a needle to inject cardiac muscle cells or undifferentiated satellite cells for cellular cardiomyoplasty, inserting a cannula to inject pharmacological agents for angiogenesis, robotic, cutting, stabilizing and anastomotic instruments for performing coronary artery bypass or coronary artery bypass grafting, or positioning a laser or other energy probe or mechanical piercing element to pierce the heart muscle for transmyocardial revascularization, or placing bipolar electrodes, or ablating epicardial tissue for treatment of atrial fibrillation or installing supports or constraints to inhibit distention of the heart. In addition, the atrial appendage may be ligated and transected to prevent release of emboli in atrial fibrillation, for example, by advancing a suture loop through the endoscopic cannula to cinch off the atrial appendage to prevent blood clots, which frequently form in the appendage, from migrating out and traveling to the brain.

Once an opening 615 has been formed in the pericardium, the cannula 700 may be advanced through the opening to access the heart. The pericardial entry instrument may be removed from the working lumen, and a variety of instruments may be inserted through the working lumen to perform procedures on the heart. For example, a probe may be advanced through the working lumen to perform epicardial ablation for cardiac arrhythmias, including atrial fibrillation or ventricular tachyarrhythmias. A radiofrequency probe or laser or a simple mechanical probe may be used to pierce the myocardium in multiple sites for transmyocardial revascularization (TMR). A needle may be advanced through the working lumen to inject undifferentiated muscle cells into infarcted areas of the heart in the procedure of cellular cardiomyoplasty. Angiogenic pharmacologic agents may be injected into the myocardium. Devices may be inserted through the working lumen. A cardiac reinforcement device, for example, as described in U.S. Pat. Nos. 6,077,218 and 6,085,754 and improvements thereof, may be inserted through the working lumen to surround the heart and restrict its volume in congestive heart failure. A linear stapler or a suture loop may be applied to the base of the atrial appendage, to seal off its opening and prevent ejection of blood clot into the cerebral circulation in patients with chronic atrial fibrillation.

In surgical procedures such as described above, the transparent tip 104 performs the role of retracting the pericardium from the epicardial surface of the heart, to allow visualization of the instrument inserted through the working lumen, and also allowing continuous endoscopic visualization of the desired area of the heart, as the instrument is guided to perform the respective cardiac procedure.

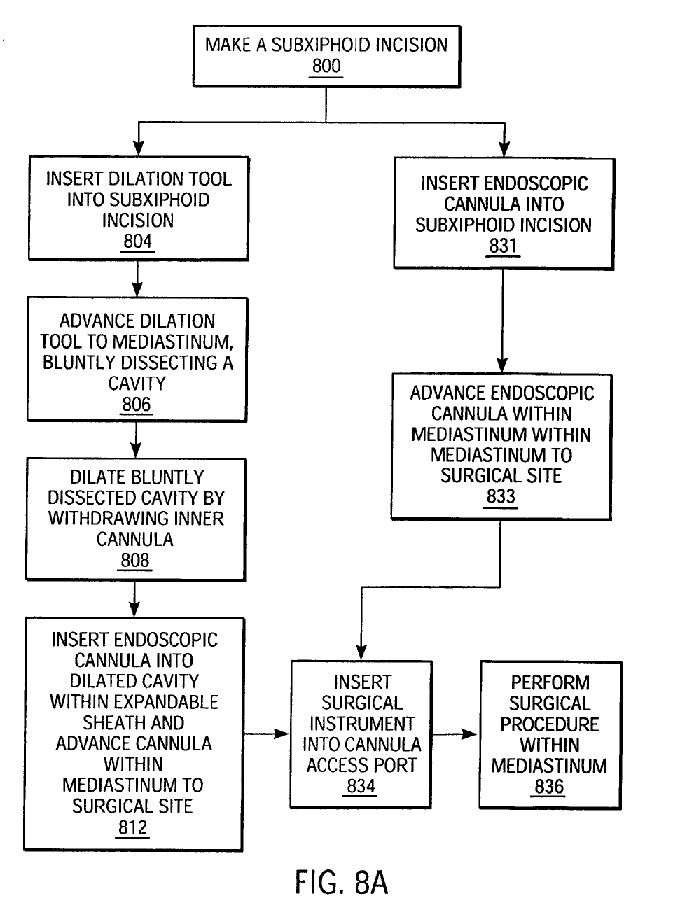

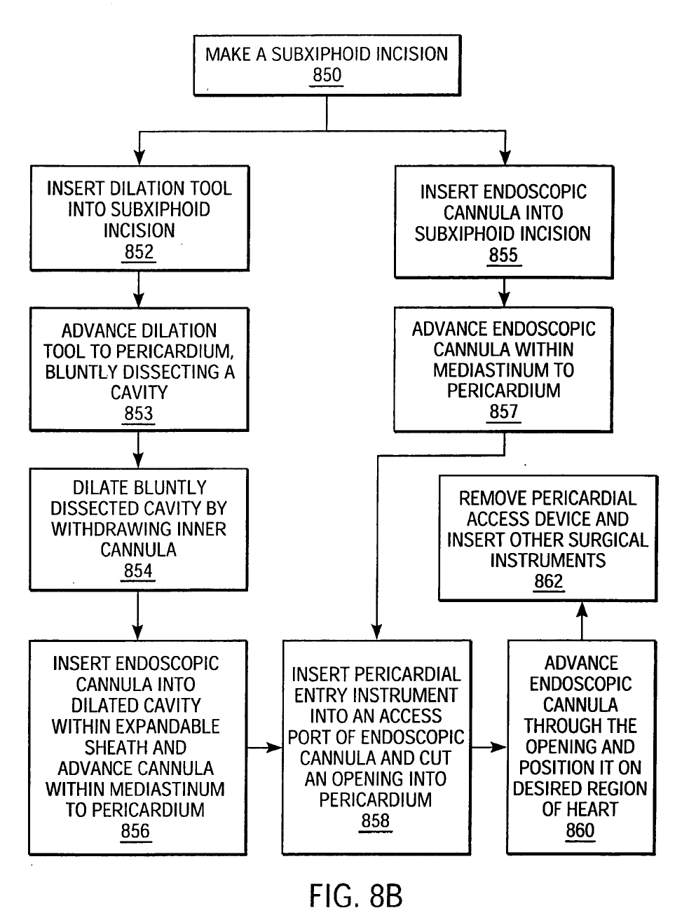

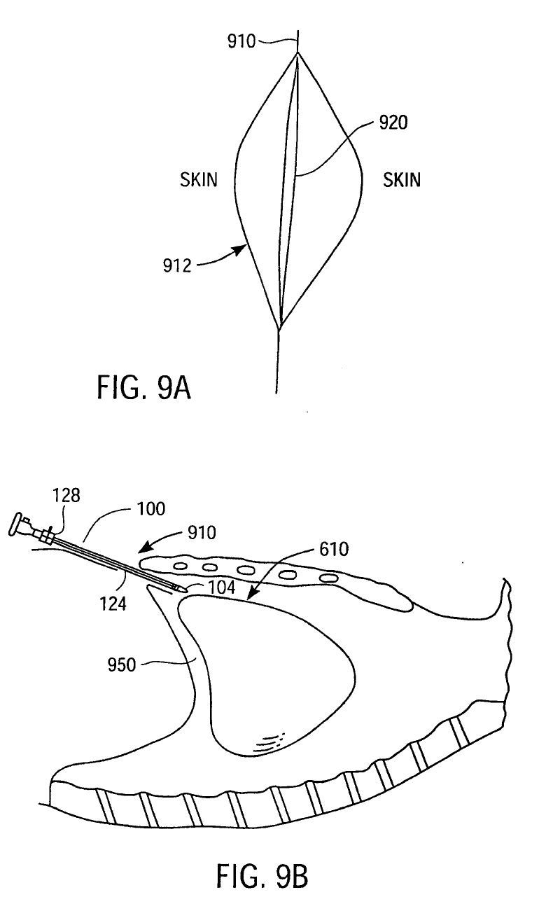

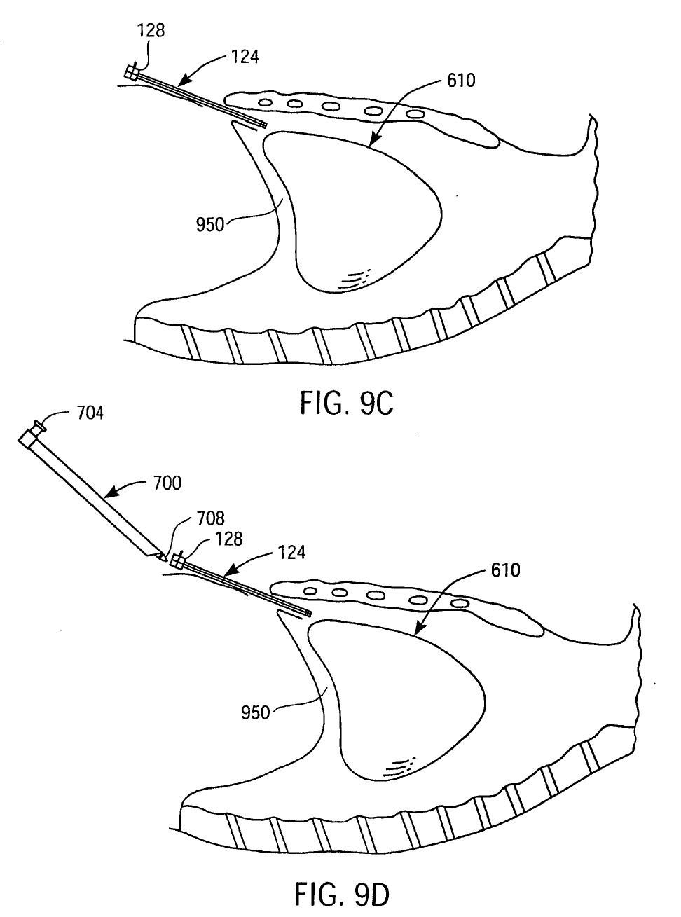

FIGS. 8A and 8B illustrate methods of performing surgical procedures in accordance with the present invention using the devices described above, and will be described with reference to FIGS. 9A-D and 10A-D. FIGS. 8A and 9A-D illustrate a method of performing surgery on mediastinal structures in accordance with the present invention. For a pericardial procedure, an incision 912 is made below the xiphoid process 910 (referred to as a subxiphoid incision 800) overlying the entry site, and the linea alba 920 is incised according to conventional practice, as shown in FIG. 9A. Next, dilation tool 100 of the present invention is inserted 804 into the subxiphoid incision under endoscopic visualization. The dilation tool 100 is advanced 806 to the mediastinum 950 under endoscopic visualization, as shown in FIG. 9B. Advancement of dilation tool 100 causes tapered tip 104 to bluntly dissect a cavity as dilation tool 100 is advanced through tissue. Dilation tool 100 is then positioned within the bluntly dissected cavity in the mediastinum 950 on the posterior aspect of the xiphoid process and sternum, for example to a position with tip 104 facing the pericardium 610 (but alternatively to a position in which tip 104 faces another organ within the mediastinum), as shown in FIG. 9B.

As the dilation tool 100 has a relatively small diameter, its use before the advancement of larger diameter instruments minimizes the risk of trauma to the surgical site. The bluntly dissected cavity created in steps 804 and 806 is dilated 808 by withdrawing inner cannula 108 through expandable sheath 124 of dilation tool 100, leaving sheath 124 in place as shown in FIG. 9C. Retraction of inner cannula 108 with enlarged region 118 through the length of expandable sheath 124 dilates the tissue adjacent to the length of expandable sheath 128 to at least the maximal dimension of the enlarged region 118. The rigid slide mount 128 is held in place while the inner rigid cannula 108 is pulled back or is withdrawn. The proximal taper 116 of cannula tip 104 rides against the chamfered inner surface of the distal end of the expandable sheath 128 to ease the initial process of cannula removal.

The generally rigid structure of the split shells radially displaces the surrounding tissue as the shells part or separate, thus dilating the cavity initially created by advancement of dilation tool 100. Substantially all of the force resulting from withdrawing cannula tip 108 is exerted on the inner surfaces of the shells 136, and not on the surrounding tissue. However, in accordance with the present invention, only radial force is exerted on the tissue by the split shells 136, which reduces any trauma to the tissue from the dilation process. The dilation of the cavity facilitates subsequent insertion into the lumens of larger diameter instruments, particularly the endoscopic cannula of the present invention, as shown in FIG. 9C.

As shown in FIG. 9C, expandable sheath 124 stays in place after withdrawing inner cannula 108. A larger diameter instrument, for example the endoscopic cannula 700 of the present invention, is inserted 812 into the cavity dilated by expandable sheath 124, as shown in FIG. 9D. Surgical instruments are inserted 834 into the one or more access ports or lumens of endoscopic cannula 700, for example access port 718 as shown in FIG. 7C. Surgical procedures are then performed 836 within the mediastinum 950 on the desired mediastinal organ. Typical surgical procedures that may be performed in the mediastinum include, for example, removal or biopsy of lymphatic glands, thymectomy (removal of thymus gland), tracheal and esophageal repair in addition to the surgical procedures previously described herein. Typical surgical instruments that may be inserted for operation in the mediastinum include ablation catheters, radiofrequency or cryogenic probes, biopsy needles, and endoscopic graspers, shears and needle holders.

Alternatively, the mediastinum 950 may be accessed without initially dilating a cavity using dilation tool 100, as shown in the alternative flow chart in FIG. 8A. A subxiphoid incision is made 800 overlying the entry site, and the linea alba 920 is incised according to conventional practice. Next, a larger diameter surgical tool (for example the endoscopic cannula 700 of the present invention) is inserted 831 into the subxiphoid incision and positioned in the mediastinum on the posterior aspect of the xiphoid process and sternum. Larger diameter surgical tools are advanced 833 in the mediastinum 950 to the surgical site of interest under endoscopic visualization, thereby bluntly dissecting a cavity responsive to its advancement. Surgical instruments are inserted 834 into an access port of the larger diameter surgical tool, for example access port 718 of the endoscopic cannula 700 of the present invention. The surgical instruments may be advanced either concurrently or sequentially, as needed to be inserted, used, then retracted, followed by a second instrument inserted, used, and retracted. Finally, the surgical procedure 836 is performed within the mediastinum 950 on the desired mediastinal organ.

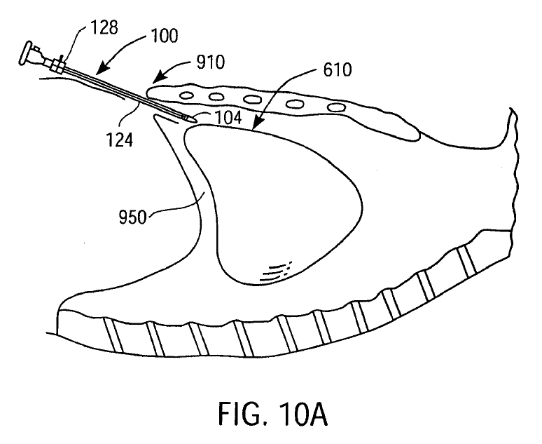

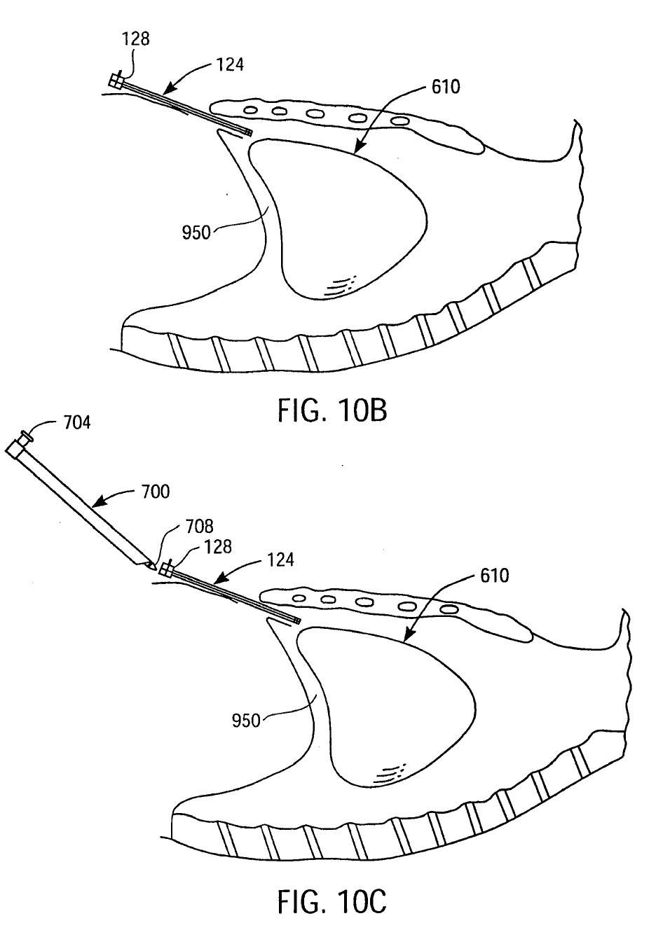

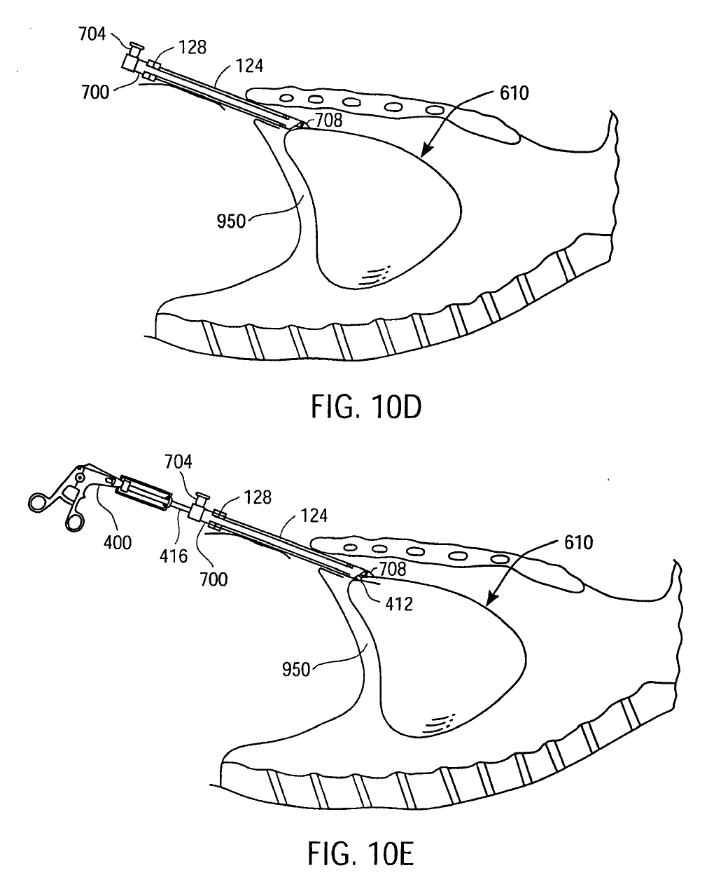

When the mediastinal organ of interest is the heart (situated within the pericardium), the surgical procedure method is generally as described above until the larger diameter instrument reaches the pericardium. Referring now to FIGS. 8B and 10A-E and 11A, a subxiphoid incision 850 is made and the linea alba is incised according to conventional practice, as shown in FIG. 9A. Dilation tool 100 is inserted 852 into the subxiphoid incision under endoscopic visualization as shown in FIG. 10A, and a cavity is bluntly dissected 853 during its advancement. The cavity is dilated 854 as previously described using the dilation tool as shown in FIG. 10B. The larger diameter instrument (for example endoscopic cannula 700 of the present invention) is advanced 856 within the mediastinum 950 toward the pericardium through the dilated cavity under endoscopic visualization as shown in FIG. 10C. Alternatively, the endoscopic cannula is advanced 855, 857 directly into the subxiphoid incision without first dilating the bluntly dissected cavity.

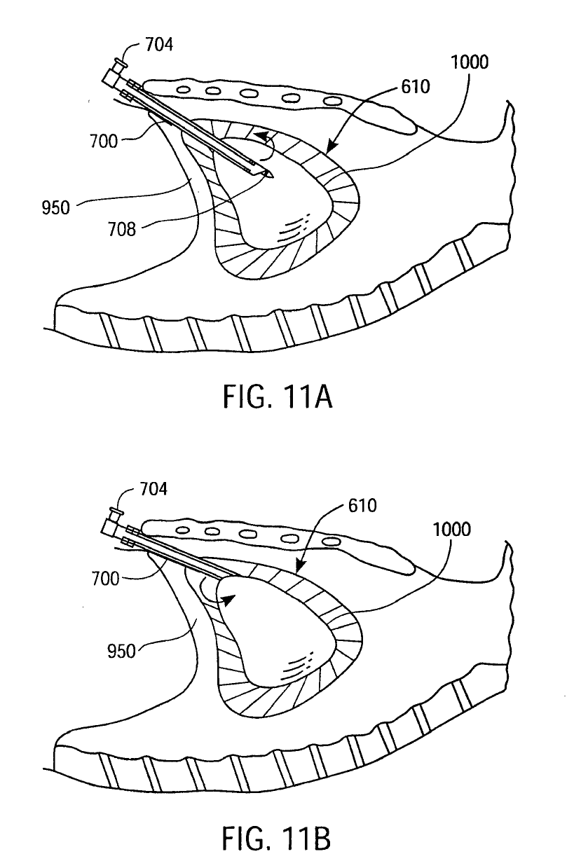

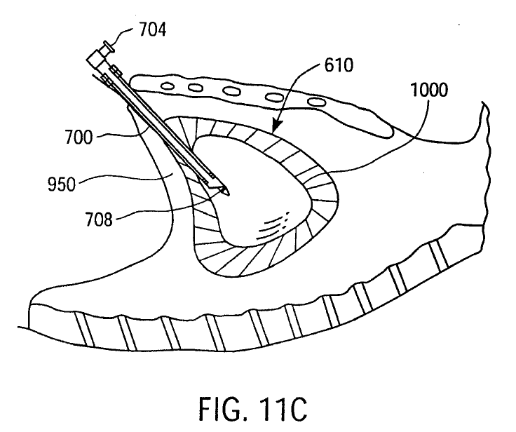

Upon reaching the pericardium as shown in FIG. 10D, an opening is cut in the pericardium 858 using the pericardial entry instrument as previously described and as shown in FIG. 10E. Specifically, as shown in FIG. 4, for a pericardial entry, the anterior pericardium is grasped with pericardial entry instrument 400 to lift the pericardium away from the heart. Tubular cutter 408 is then rotated to create a controlled cut of the pericardium, creating opening 615. Endoscopic cannula 700 is advanced 860 through the opening and is positioned on the desired region of the heart under endoscopic visualization (FIG. 11A). Preferably, opening 615 is made near the apex of the pericardium and endoscopic cannula is initially advanced from the apex toward the base of the heart. The left anterior descending coronary artery and the left atrial appendage provide landmarks for the surgeon so the location of the surgical site of interest is more easily found.

The pericardial entry 400 instrument is removed 862 from access port 718 of endoscopic cannula 700, and other desired surgical instruments are inserted through access port 718 to operate on the heart within the pericardium. In an alternative embodiment, endoscopic cannula 700 includes more than one access port and removal of the pericardial entry instrument is not necessary for the insertion of other surgical instruments. In still another embodiment, the access port is of a sufficient size that several surgical instruments may be inserted concurrently. The surgical and therapeutic operations which can be performed at this point include but are not limited to such procedures as were previously described herein. In addition, the atrial appendage may be ligated and transected as previously described herein to prevent embolism in patients with chronic atrial fibrillation, for example by advancing a suture loop through the endoscopic cannula to cinch off the atrial appendage to prevent migration of blood clots which frequently form in the appendage from migrating out and traveling to the brain or other organs.

The subxiphoid pericardial access method as herein described is particularly advantageous as it enables the surgeon to access all regions of the heart, that is 360-degree access including the anterior, posterior, left and right regions of the heart. Referring now to FIGS. 11A-C, endoscopic cannula 700 is initially inserted into the pericardium 610, preferably via an incision near the apex of the heart 1000, and then swept around the heart 1000 over the anterior and posterior surface of the heart 1000 (e.g. from the position shown in FIG. 11A to that shown in FIG. 11B and then back to the position shown in FIG. 11C). As shown in FIGS. 11A-C, endoscopic cannula 700 is maneuvered around the heart 1000 in such a way that all regions of the heart may be accessed. The endoscopic cannula can be maneuvered because of the subxiphoid entry position and the flexibility of soft tissue around the heart, the softness of the tissue allowing the endoscopic cannula to push apart tissue and move around the heart. Thus, all regions of the heart may be accessed without the need for invasively lifting or rotating the heart to access posterior or lateral vessels and structures.

As described above, once a larger diameter instrument, for example endoscopic cannula 700, is inserted into the pericardium (either through a cavity dilated by expandable sheath 124, as shown in FIG. 9D, or without using an expandable sheath, as shown in FIGS. 11A-11C), surgical instruments are inserted into the one or more access ports or lumens of the larger diameter instrument, for example, port 718 of endoscopic cannula 700 as shown in FIG. 7C.

The several apparatus of the various aspects of the present invention have been discussed in relation to a subxiphoid access surgical method. However, uses of the apparatus disclosed herein including an endoscopic cannula, a dilation tool, and a pericardial entry instrument, are not limited to use with the subxiphoid access method. While the subxiphoid access method is preferred because of its minimally invasive nature, other methods of access may also be used, for example, via an incision in the intercostal region and advancing the endoscopic cannula through the incision to gain access to the pleural cavity. In such a procedure, the pleural membrane and the pericardial membrane, which lie in contact with one another, are grasped and punctured using the pericardial entry instrument to reach the heart. In addition, the methods described herein are not limited to accessing mediastinal structures (which includes the pericardium). For example, procedures requiring access to the peritoneum, the dura mater, or any membrane overlying a sensitive organ, for example the spine, the brain, or the stomach, also benefit from the use of the apparatus and method described above. Additionally, the method and apparatus described above may also be employed in procedures requiring access to the saphenous vein, radial artery, internal mammary artery, the peritoneum, the dura mater or through any membrane overlying a sensitive organ such as the spine, the brain or the stomach.

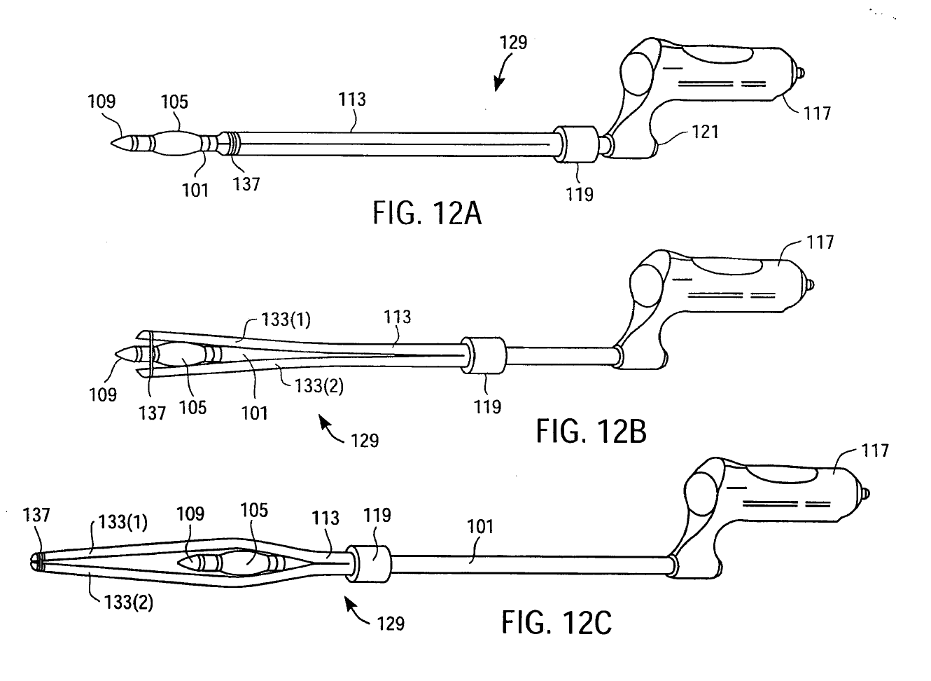

Referring now to FIG. 12A, there is shown a perspective view of another longitudinal mechanical dilator 129 in accordance with the present invention which comprises an inner cannula 101 and an outer expandable sheath 113. A tissue expansion device 105 is disposed on the distal end of the inner cannula 101. The outer expandable sheath 113 is preferably split longitudinally into two shells 133(1) and 133(2). In one embodiment, the distal end of the outer expandable sheath 113 is compressed against the outer surface of the inner cannula 101 by a resilient connector 137. The proximal end of the outer expandable sheath 113 includes an integrated segment 119, for example, near or within a handle 117. Thus, upon retracting the tissue expansion device 105 through the distal end of the outer expandable sheath 113, as shown in FIG. 12B, the tissue expansion device 105 exerts an outward force against the outer expandable sheath 113 which facilitates expansion of the resilient connector 137. As shown in FIG. 12C, the tissue expansion device 105 is then retracted toward the proximal end of the expansible sheath 113, pushing the shells 133 outward and thus dilating any surrounding tissue. Further movement of the tissue expansion device 105 in the proximal direction is restrained upon reaching the integrated end 119 of the expandable sheath 113.

In one embodiment, the longitudinal mechanical dilator 129 may be used for vessel harvesting procedures under endoscopic visualization. In this embodiment, the inner cannula 101 has an endoscopic lumen 121 for housing an endoscope and has a transparent tip 109 for viewing therethrough. The transparent tip 109 is tapered to provide improved visualization and dissection capabilities. The tissue expansion device 105 may be formed as a wedge or in an olive shape, and may be made of a rigid or semi-rigid material such as rubber, Teflon, polyurethane, polycarbonate, or the like. One preferred wedge or olive is described in co-pending application Ser. No. 09/413,012 entitled “Tissue Dissection Apparatus and Method”, filed Oct. 10, 1999. The tissue expansion device 105 is situated near or immediately proximal to the tip 109 of the dilator 129. The tissue expansion device 105 may be formed as an integral part of the tip 105, or may be formed independent of the tip 105 as part of the elongated body of the cannula 101. The cannula 101 may be substantially rigidly formed to provide the support for the axial force exerted against the expandable sheath 113. The cannula 101 may be made from a variety or combination of bioinert, substantially inelastic materials, such as stainless steel, polyethylene, polyurethane, polyvinyl chloride, polyimide plastic, and the like that preferably have a tensile strength of at least 10,000 psi. Handle 117 is ergonomically formed to allow a surgeon to easily and comfortably manipulate cannula 101 within a surgical cavity.

The expandable sheath 113 includes a solid or rigid segment 119 near the proximal end, as described above, although alternatively the sheath 113 may comprise two independent shells that are coupled together at their proximal ends. The solid or integrated segment 119 may be of an increased diameter to serve as a separate handle for convenient gripping by a surgeon. For example, when the surgeon retracts the inner cannula 101, the surgeon may grip the segment 119 to maintain the outer expandable sheath 113 at the location where dilation is desired. In one embodiment, the outer diameter of the tissue expansion device 105 combined with the outer diameter of the expandable sheath 113, and any added outer elastic covering (not shown, for clarity), are selected to permit the longitudinal mechanical dilator 129 to fit through a standard 12 mm diameter gas insufflation port, as vessel dissection is typically performed with concurrent gas insufflation. In this embodiment, as the tissue expansion device 105 is pulled in a direction toward the proximal integrated end 119, the sheath 113 expands to approximately a 20 mm outer dimension. In embodiments in which gas insufflation is not used, or in embodiments in which the ports are of different sizes, the sizes of the components of the dilator 129 may be adjusted accordingly.

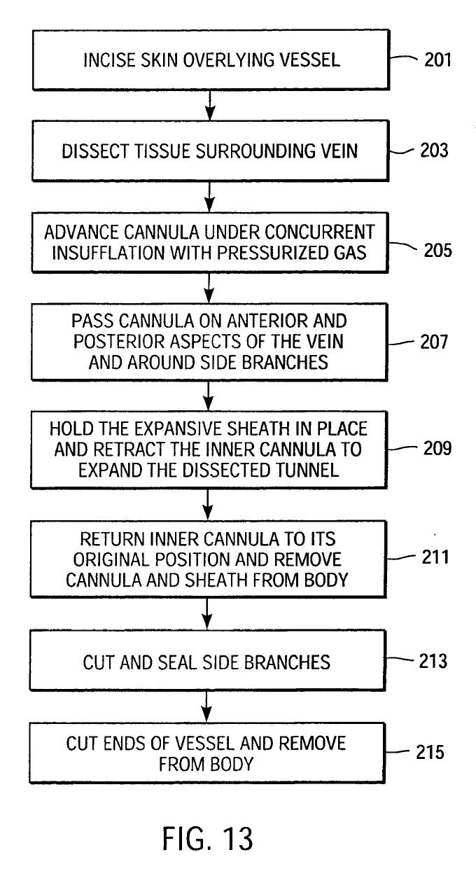

FIG. 13 is a flow chart illustrating a method of dilating tissue in accordance with the present invention, specifically with respect to harvesting a vein as one example. First, the surgeon makes a small incision 201 in the skin overlying the vessel of interest, for example, the saphenous vein. Then, the surgeon bluntly dissects 203 connective tissue covering the vein to expose the adventitial surface of the vein. The surgeon advances 205 a cannula with a transparent tapered tip disposed at the distal end in contact with the adventitial surface of the vein under endoscopic visualization through the transparent tip, and optionally under concurrent insufflation of the tunnel with pressurized gas to dissect an initial tunnel along the vein. At this stage in the procedure, the longitudinal mechanical dilator 129, a conventional endoscopic cannula with a transparent tapered tip, or any other instrument for initially dissecting a tunnel may be used in accordance with the present invention. The insufflation of the tunnel provides additional dilation and helps maintain the shape of the tunnel when the device is withdrawn. Then, the surgeon passes 207 the tip of the cannula along the anterior and posterior aspects of the vein and around the side branches to dissect a tunnel along the selected length of the vein. If a device other than the longitudinal mechanical dilator 129 of the present invention is being used, such other device is withdrawn and the longitudinal mechanical dilator 129 is inserted into the incision. If the longitudinal mechanical dilator 129 is being used to dissect the initial tunnel, then it is advanced to the end of the dissected perivascular tunnel under endoscopic vision through the transparent tip 109, and, holding the integrated 119 of the expandable sheath 113 stationary, the surgeon pulls or retracts 209 the tissue expandable device 105 on the inner cannula 101 through the expansible sheath 113 to expand the shells 133 and thereby further dilate tissue in the dissected tunnel. The zone of expansion corresponds to the region of the expandable sheath 113 under which the tissue expansion device lies. This zone extends from the distal to the proximal end of the tunnel as the tissue expansion device 105 is pulled in the direction distally to proximally. Thus, an evenly-shaped zone of expansion is formed by the retraction of the tissue expansion device 105 through the expandable sheath 113. Additionally, the dilation may be generated by one smooth motion of pulling the inner cannula 101 through the sheath 113, as previously described, and thus the repetitive motions of conventional systems are avoided. Finally, the size of the tissue expansion device 105 and the rigidity of the shells 133 create a sufficiently large tunnel within which additional instruments can be maneuvered.

After the tunnel is dilated, the surgeon returns 211 the tissue expansion device 105 to its original position to contract the expansible sheath 113 for convenient removal of the dilator 129 from the body. Contracting the expandable sheath 113 prior to removal minimizes the trauma to surrounding tissue caused by the longitudinal mechanical dilator 129. Then, the surgeon inserts additional instruments within the dilated tunnel to seal or apply clips and cut 223 the side branches of the vessel to be harvested. Finally, the surgeon cuts the two ends of the vessel and removes 215 the target vessel from the body.

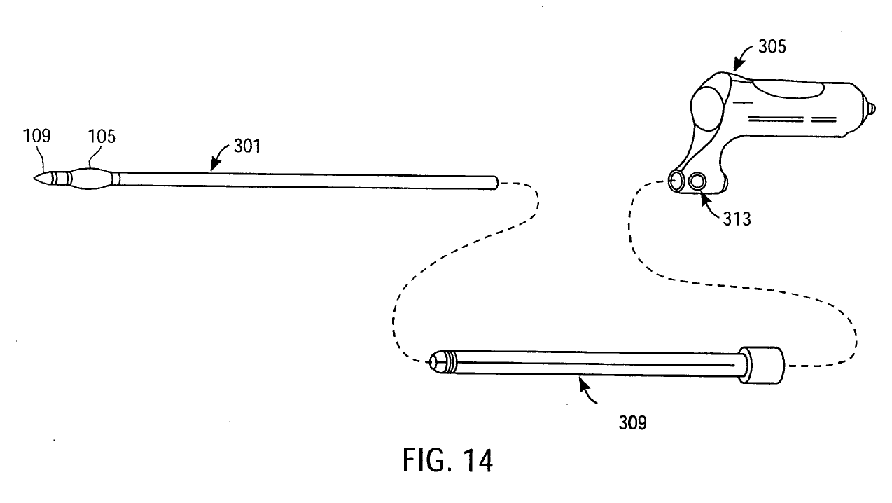

FIG. 14 is a perspective, exploded view illustrating an alternate embodiment of a longitudinal mechanical dilator in which an expandable sheath is removable from an inner cannula. In this embodiment, the inner cannula 301 detaches from the handle 305 to allow the expandable sheath 309 to be removed from and added to the inner cannula 301 and handle 305, as desired. This embodiment provides a dissection cannula 301 of a smaller outer diameter along the majority of its length with the exception of the region of the tissue expansion device 105. Thus, this dissection device 301 may be used to provide initial dissection as described above in connection with FIG. 13, with increased tip maneuverability due to the small diameter of the cannula 301 for dissecting the vessel from the surrounding connective tissue. In one embodiment, the expandable sheath 309 is made removable by attaching a locking mechanism 313 to the handle 305. To remove the sheath 309, the end of the inner cannula 301 is unlocked from the handle 305 and the sheath 309 is removed by sliding the sheath 309 in a proximal direction and off the inner cannula 301. To place the sheath 309 on the inner cannula 301, the handle is unlocked and removed 305, the sheath 309 is slid onto the cannula 301, and the handle 305 is locked back into place. In one embodiment, the locking mechanism 313 is a threaded thumbscrew that fixes the proximal end of the inner cannula 301 in place upon being tightened against the inner cannula 301.

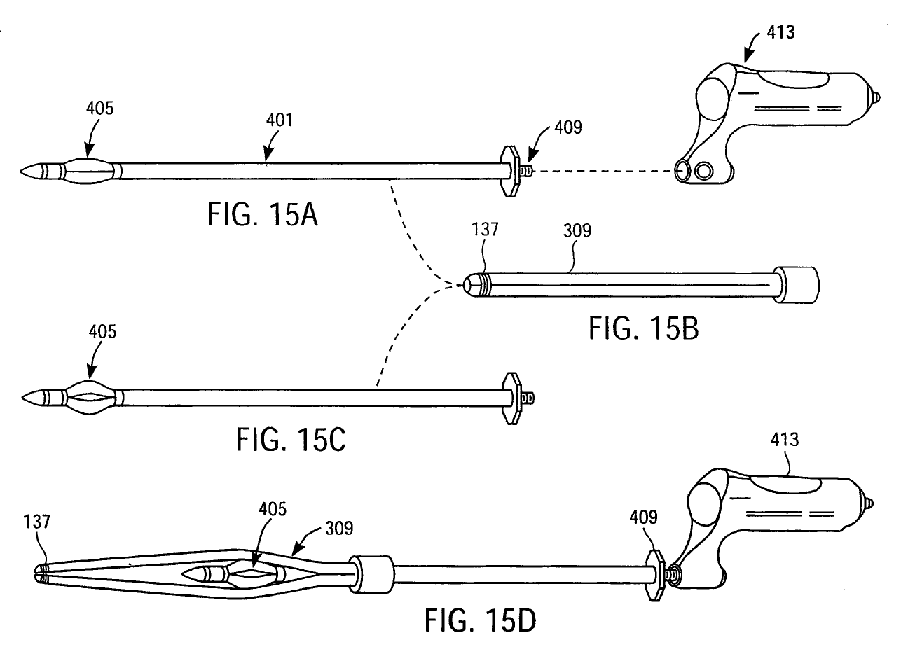

FIG. 15A illustrates another embodiment of the longitudinal mechanical dilator of the present invention that provides two-stage dilation. In one embodiment, a tissue expansion device 405 is split longitudinally into two or more sections as shown in FIGS. 15B and 15D and an axial compressor mechanism 409, in one embodiment including a threaded shaft as later described herein, compresses the tissue expansion device 405 when dilation is sought to cause the split tissue expansion device 405 to expand. Thus, the split tissue expansion device 405 remains in a closed or compact configuration having a minimal outer diameter when dilation is not required, and then can be expanded to a greater outer diameter when dilation is required. In one embodiment, the inner cannula 401 extends back to the handle 413, and a proximal portion of the inner cannula 401 is externally threaded. In this embodiment, the axial compressor 409 is a threaded nut that is positioned on the proximal end of the inner cannula 401. Other mechanisms such as a toggled lever for compressing the tissue dilation device 405 may also be used in accordance with the present invention. Upon rotating the threaded nut, the distal end of the inner cannula 401 adjacent the proximal end of the split tissue dilation device 405 exerts an axially-directed force against the split tissue dilation device 405. The distal end of the split tissue dilation device 405 is fixably attached to the inner cannula 401 and the proximal end is slidably attached. Therefore, as the distal end of the inner cannula 401 presses against the split tissue dilation device 405, the dilation device 405 is compressed and expands in diameter as shown in FIG. 15C. The expanded tissue expansion device 405 is retracted through the outer expandable sheath 309, as shown in FIG. 15D, to expand the outer dimension of the sheath 309 to a greater dimension that may exceed 20 mm. Thus, this embodiment provides a cannula 401 that dissects an initial tunnel with increased maneuverability and minimal applied force. However, by adding the outer expandable sheath 309 and compressing the tissue dilation device 405, the instrument 401 can be used to dilate a large tunnel within the tissue.

The present invention has been described above in relation to vessel harvesting. However, it should be noted that the apparatus and method of the present invention may also be utilized in procedures, for example, requiring access to the peritoneum, the dura mater, or other organ such as the heart through tissue that requires dissection and dilation along an access channel.

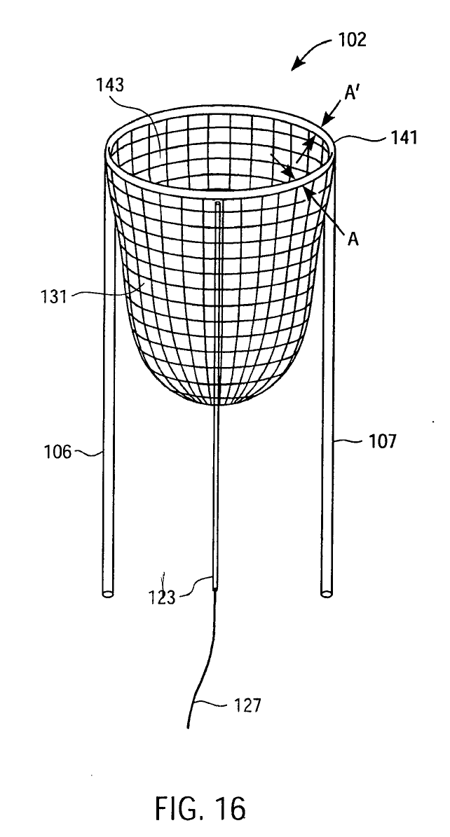

Referring now to FIG. 16, there is shown cardiac restraint apparatus 102 which embodies an aspect of the invention. Cardiac restraint apparatus 102 comprises jacket 131 and rim 141 that defines opening 143 sufficiently large to receive a heart. Jacket 131 is attached to rim 141 along substantially the entire perimeter of the open end of jacket 141. The apparatus further comprises knot pusher 123 and strand 127 having an end which extends through knot pusher 123. The apparatus also includes guide tubes 106 and 107, removably attached to rim 141. Strand 127 extends around rim 141.

Jacket 131 can be constructed of a wide variety of materials, but generally it should be constructed from materials that are biocompatible and non-toxic to bodily tissue, for example distensible or non-distensible mesh fabric constructed from silicone rubber, nylon, polyurethane, polyester, polytetrafluoroethylene (PTFE), expanded PTFE (ePTFE), polypropylene, stainless steel, and impregnated elastomers such as nylon in polyurethane or nylon in silicone rubber. While FIG. 16 illustrates jacket 131 as being open at one end and closed at the other, the invention also contemplates a jacket or band that is open at both ends.

Rim 141 is hollow, for example constructed as a hollow tube or a folded fabric sleeve, which is capable of receiving and containing strand 127. Rim 141 may be constructed separately from any biocompatible, flexible material (such as biocompatible fabrics and plastics) and attached to jacket 131 around the perimeter of opening 143, or may alternatively be constructed by simply folding and securing the mesh fabric of jacket 141 around opening 143 to create a hollow fabric sleeve.

Knot pusher 123 can be constructed from any suitable material capable of being formed into a hollow tube, for example, rigid and flexible plastics or metals such as stainless steel.

Strand 127 can be constructed from any conventional surgical suture material, for example nylon, silk, steel, catgut, and conventional bioabsorbable suture materials such as polymers and copolymers of lactide, glycotide, para-dioxanone and trimethylene carbonate. At least one end of strand 127 is disposed within knot pusher 123. As used in the present invention, the term “strand” includes any of a variety of strings, fibers, wires, or sutures capable of being tied into a slipknot.

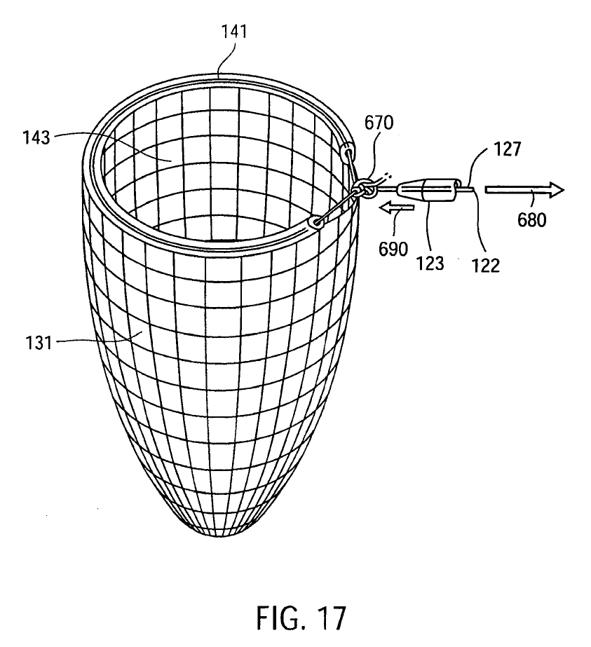

FIG. 17 illustrates the structural relationship between knot pusher 123, rim 141 and strand 127. In this figure, guide tubes 106 and 107 have been omitted for clarity. At the juncture where knot pusher 123 meets rim 141, strand is tied into slipknot 670. At least one end 122 of strand 127 is disposed within knot pusher 123, which in this figure is illustrated as having, optionally, a tapered distal end. The operation of knot pusher 123 is illustrated by arrows 680 and 690 in FIG. 17. Strand 127 is pulled away from the heart in the direction of arrow 680 (proximally) while knot pusher 123 is pushed against the slipknot in the direction of arrow 690 (distally). The distal movement of knot pusher 123 pushes knot pusher 123 against slipknot 670, holding slipknot 670 while pulling strand 127 away from the heart and causing a reduction of the diameter of opening 141, thereby tightening jacket 131 around the heart (not shown).

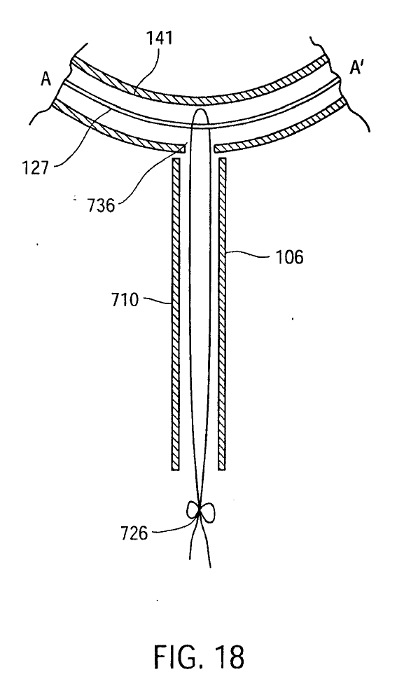

Referring again to FIG. 16, the illustrated embodiment of a cardiac restraint apparatus according to the invention also includes one or more guide tubes 106 and 107 that are removably attached to rim 141. Guide tubes 106 and 107 may be attached by any suitable detachable means, for example by having perforations at the site of attachment. Alternatively, the guide tubes 106 and 107 may be removably attached to the rim 141, as described herein with reference to FIG. 18.

Referring now to FIG. 18, there is shown a partial cross sectional view of a portion of the rim of a jacket of a cardiac restraint apparatus according to one embodiment of the present invention. In this embodiment, rim 141 includes an opening 736 at the site where a guide tube 106 meets rim 141. Connecting strand 710 extends within guide tube 106, is looped over strand 127 (strand 127 extends within and around rim 141), and is tied into knot 726. Guide tube 106 is removable by cutting connecting strand 710 or unraveling knot 726 and disengaging connecting strand 726 from strand 127, thereby disengaging guide tube 106 from rim 141. Guide tubes 106 and 107 can be constructed from any suitable material capable of being formed into a hollow tube, for example rigid and flexible plastics or metals such as stainless steel. Preferably guide tubes 106 and 107 have a diameter of about 1 mm to 1.5 mm.

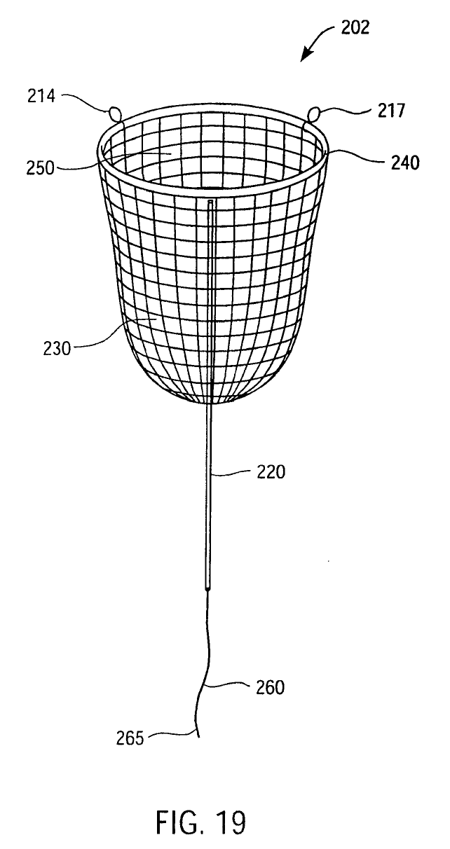

An alternative embodiment of a cardiac restraint apparatus according to the present invention is illustrated in FIG. 19. Cardiac restraint apparatus 202 is similar to the cardiac restraint apparatus 102 of FIG. 16, except that guide tubes are replaced by at least one handle 214 for guiding the apparatus during performance of a surgical procedure. Thus, the guide tubes 106 and 107 shown in FIG. 16 and the handles 214 and 217 shown in FIG. 19 are alternative embodiments of guide elements to help in guiding the placement of the cardiac restraint apparatus around the heart during surgery. Specifically, this alternative embodiment of a cardiac restraint apparatus according to the invention comprises jacket 230 and rim 240, the rim 240 defining opening 250 sufficiently large to receive a heart. Jacket 230 is attached to rim 240 along substantially the entire perimeter of the open end of jacket 240. The apparatus further comprises knot pusher 220 and strand 260 having end 265 which extends through knot pusher 220 and extends around rim 240. The apparatus also includes handles 214 and 217 attached to rim 240.

Handles 214 and 217 may be constructed from any conventional surgical suture material, for example nylon, silk, steel, catgut, and conventional bioabsorbable suture materials such as polymers and copolymers of lactide, glycotide, para-dioxanone and trimethylene carbonate. Handles 214 and 217 may be suitably attached to rim 240, for example, using adhesives, welding, or tying handles 214 and 217 around rim 240. Optionally, handles 214 and 217 may be removably attached to rim 240, for example by using a perforated strap (not shown).

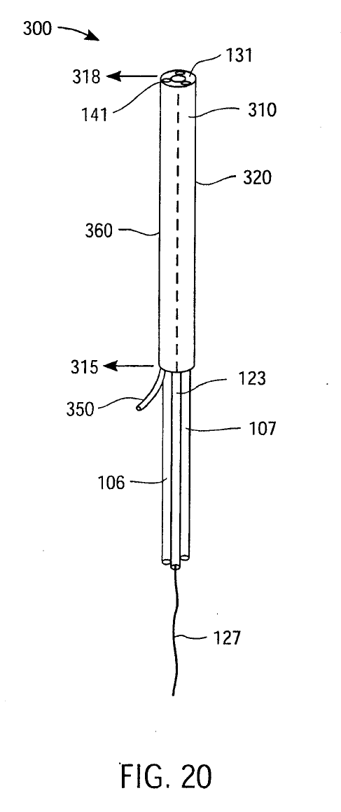

FIG. 20 is a perspective view of a sheathed embodiment of the cardiac restraint apparatus of the present invention. Sheathed apparatus 300 is the cardiac restraint apparatus illustrated in FIG. 16 that has been formed into a compact state and sheathed within sheath 320. Jacket 131 and rim 141 of apparatus 102 are folded, creased or crumpled to reduce their profile before being enclosed by sheath 320. Jacket 131 reconfigures into a non-compact state, illustrated in FIG. 16, when sheath 320 is removed.

Sheath 320 can be constructed from any flexible material, including but not limited to polyethylene, polyvinylchloride, and teflon. Sheath 320 may be of any structure suitable to enclose jacket 131 and generally includes a cylindrical body 360 having a proximal end 315 and a distal end 318 and perforations 310 along sheath body 360, and pull tab 350 attached to proximal end 315. The perforations 310 are longitudinally positioned. Sheath body 360 defines a lumen having an inner diameter of about 7 mm to 10 mm. Sheath 320 is removable from apparatus 300 by tearing sheath body 360 along perforations 310. This removal is more easily accomplished by fitting sheath 320 with a pull tab 350 extending out from the proximal end 315 of sheath body 360. Pulling tab 350 away from the apparatus 300 results in tearing of sheath body 360 along perforations 310 and removal of the torn sheath 320 from jacket 131.

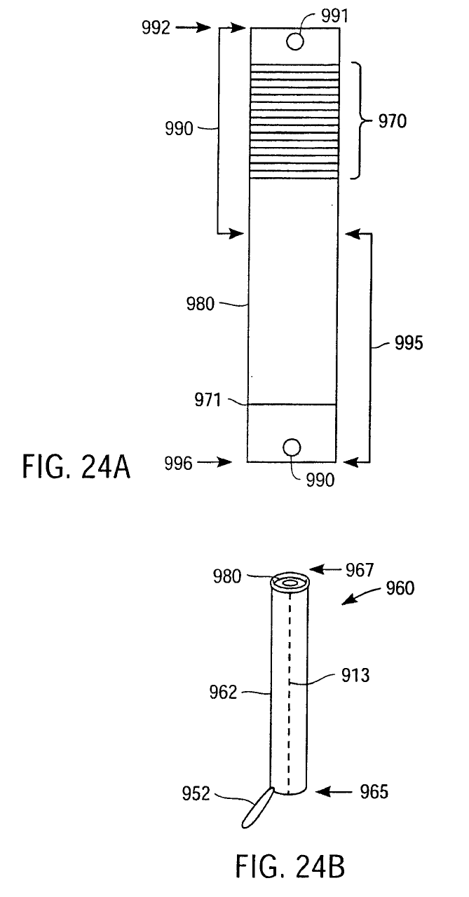

Another alternative embodiment of a cardiac restraint apparatus according to the present invention is illustrated in FIGS. 24A-24B. In this embodiment, cardiac restraint apparatus 960 comprises at least one elastic band 980 having a first portion 990 terminating at a first end 992 and a second portion 995 terminating at a second end 996, with the first portion 990 and the second portion 995 of the elastic band 980 being joined together at a location between first end 992 and second end 996. Thus, elastic band 980 may be constructed of two separate portions that have been attached together, or alternatively may be one continuous piece. Elastic band 980 may be constructed from any flexible material, including but not limited to silicone rubber, nylon, polyurethane, polyester, polytetrafluoroethylene (PTFE), expanded PTFE (ePTFE), polypropylene, and impregnated elastomers such as nylon in polyurethane or nylon in silicone rubber. Preferably, elastic band 980 has a width of about 1 cm, and a thickness of approximately 1-3 mm.

Each elastic band can be sheathed with a sheath, such as sheath 962 of FIG. 24B, when introduced into the patient. Sheath 962 has a generally cylindrical body having a proximal end 965 and a distal end 967, and can be constructed from any flexible material, including but not limited to polyethylene, polyvinylchloride, and teflon. Sheath 962 can be of any structure suitable to enclose elastic band 980 or two or more of elastic bands 980, preferably enclosing elastic band 980 in a rolled configuration as illustrated in FIG. 24B. Sheath 962 can include perforations 913 to facilitate removal of the sheath by tearing along perforations 913 that are longitudinally positioned. The sheath can also include a pull tab 952 that is attached to the proximal end 965 of sheath 962, for pulling the sheath away from the apparatus. Elastic band 980 may also include calibrated markings 970 for calibrating the tension of the elastic band 980. In use, a surgeon can calibrate the tension of elastic band 980 using calibrated markings 970 and markings 971 by stretching elastic band 980 from its relaxed state and noting the number of calibrated markings 970 overlapped by marking 971.

Optionally, the first and second ends of the elastic band 980 are configured to be engaged by a grasping instrument, for example by including openings 990 and 991 suitably sized to receive a grasping instrument.

Other aspects of the present invention include methods of restraining the heart using any embodiment of the inventive cardiac restraint apparatus. While any suitable surgical approach to the heart may be used, for example trans-xiphoid or thorascopic incisions, the preferred incision is a subxiphoid incision large enough, for example, about 2 cm, to allow for insertion of a cannula for performing minimally invasive surgery. An apparatus having a cannula through which the cardiac restraint apparatus of the present invention can be deployed, and methods of using the apparatus, are previously described herein.

Briefly, the surgical apparatus used to deploy the cardiac restraint apparatus through a subxiphoid incision is an endoscopic cannula comprising a cannula, a transparent tip located at the distal end of the cannula, and an endoscope positioned for visualizing through the distal end of the cannula. The cannula has at least one lumen, and one or more additional lumens for advancement of surgical tools therethrough. The transparent tip is tapered to provide better visualization of tissue dissection within the field of view. The transparent tip has a generally conical shape, and may be removable and replaceable, as desired to obtain clearer images of the surgical site.

In one embodiment, the endoscopic cannula may comprise one or more lumens or access ports through the cannula for receiving surgical instruments or a cardiac restraint device into a lumen of the cannula. Such endoscopic cannula further comprises an endoscopic eyepiece, skewed relative to the proximal end of the endoscope, for facilitating the viewing of a surgical site through the endoscope while minimizing interference with surgical instruments introduced into the cannula.

In accordance with one method embodiment of the present invention, the endoscopic cannula is either directly advanced to the mediastinum or, alternatively, a cavity is first dilated and the endoscopic cannula is advanced through the dilated cavity. Once the endoscopic cannula is advanced into the mediastinum, surgical tools are advanced through the one or more access ports, and surgical procedures are performed within the mediastinum, as previously described herein.

In order to restrain the heart with a cardiac restraint apparatus of the present invention using the subxiphoid method, the endoscopic cannula is advanced under endoscopic visualization, as described previously, either directly into the initial subxiphoid incision or after first dilating a cavity using a dilation tool as described herein. Upon reaching the pericardium, a flap of the pericardium is gripped and the flap is cut using a pericardial entry instrument, as described herein, to create an opening in the pericardium.

The subxiphoid access procedure enables the surgeon to access all regions of the heart, that is a 360-degree access capability including the anterior, posterior, left and right regions of the heart, but such entry is not required, and other entry positions, such as entry in the posterior region of the heart, are also acceptable. Once inside the pericardium, the cannula can be maneuvered around the heart substantially because of the subxiphoid entry and the flexibility of soft tissue around the heart. Thus, all regions of the heart may be accessed without the need for invasively lifting or rotating the heart to access posterior or lateral vessels and structures during placement of the cardiac restraint apparatus in accordance with the present invention.

The subxiphoid access procedure is performed under endoscopic visualization and is minimally invasive since only a single incision is required to gain access to all regions of the heart. In addition, as the approach is through a subxiphoid incision, there is no need to go through the pleural cavity and thus no need to deflate the lung. Conventionally, such extensive access to the heart has only been possible using invasive methods such as pericardial window, open heart surgery, or port access surgery using several incisions and ports. Thus, using the subxiphoid access method as described herein, enables a surgeon to access all regions of the heart with a single incision for most procedures, without needing to go through the pleural cavity.

The endoscopic cannula with transparent tapered tip is used directly to bluntly dissect a path to the pericardium, through the fat and connective tissue. Direct visualization allows verification that the pericardial surface is clean and devoid of adherent fat, use of the pericardial entry instrument may proceed under visual guidance on an exposed pericardial surface.

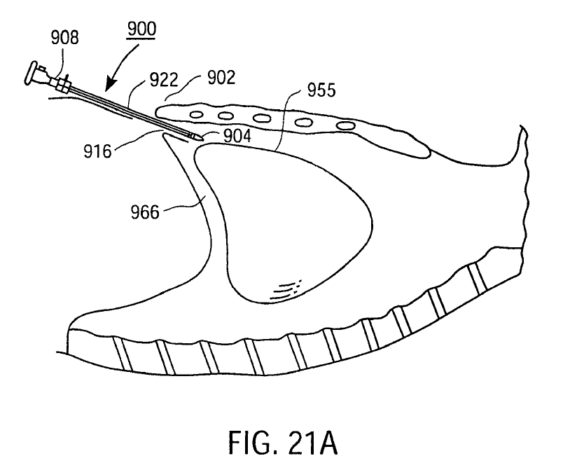

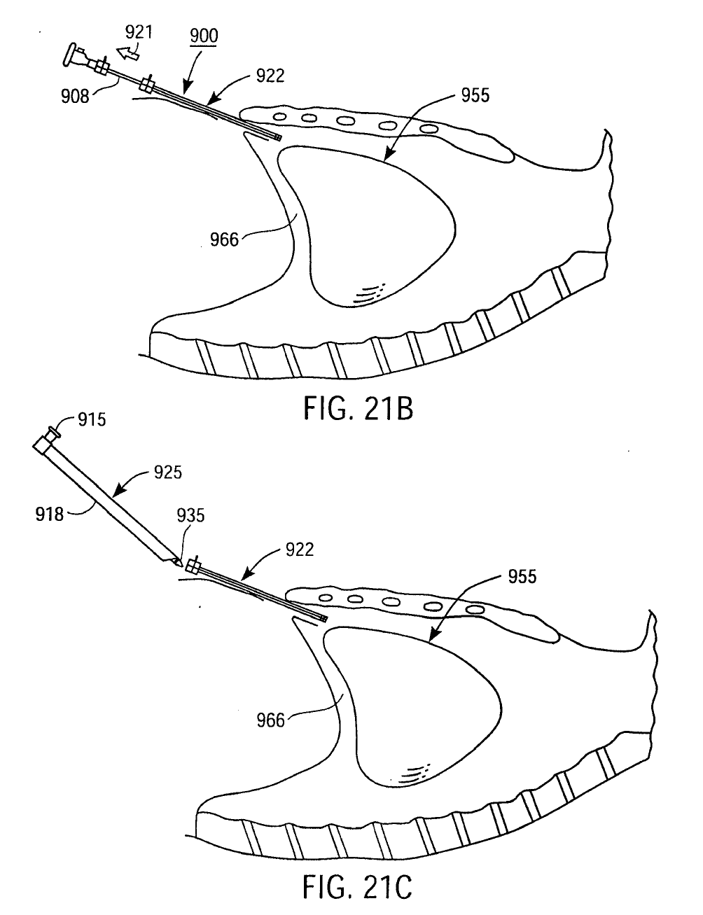

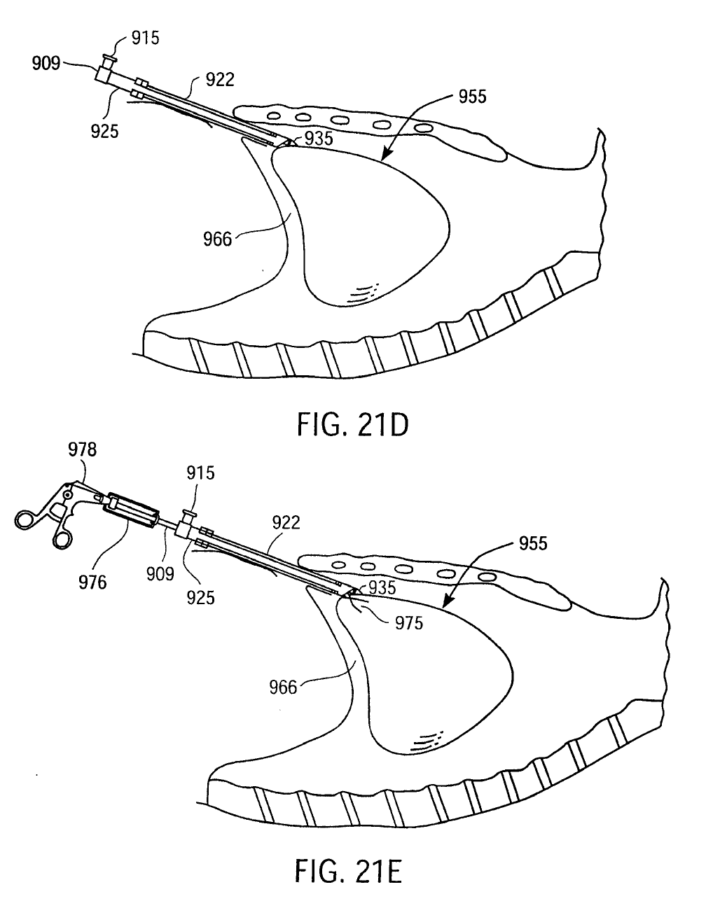

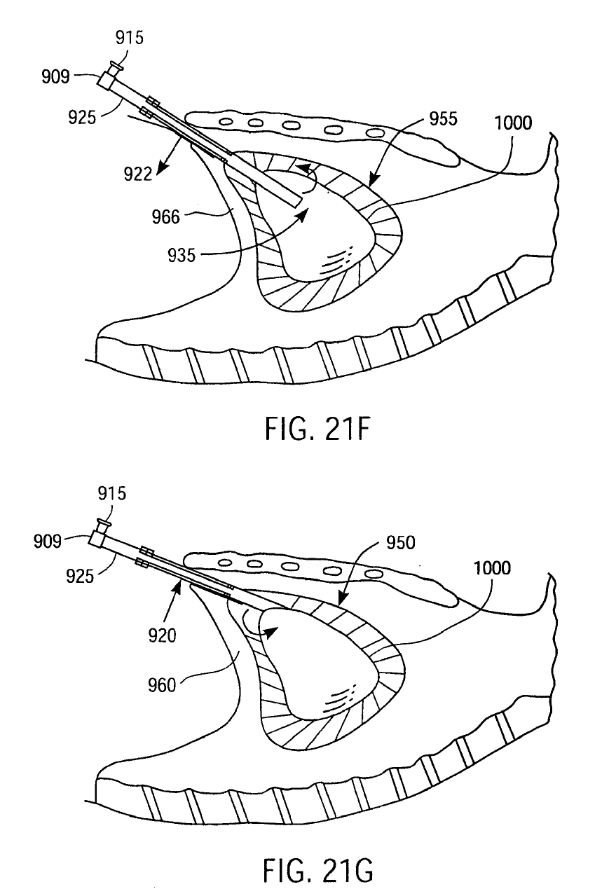

Referring now to FIGS. 21A-21G, the subxiphoid method for accessing the heart is illustrated in more detail. First, a subxiphoid incision is made overlying an entry point for a surgical procedure. The subxiphoid incision is preferably small, about 2 cm. The subcutaneous tissue below the incision is bluntly dissected to expose the linea alba, which is also incised. Referring now to FIG. 21A, dilation tool 900, comprising an inner cannula 908 having tapered tip 904 and an outer expandable sheath 922, is inserted into the subxiphoid incision 916. Tapered tip 904 of inner cannula 908 bluntly dissects a cavity responsive to the advancement of the dilation tool 900. Dilation tool 900 is then positioned on the posterior aspect of the xiphoid process 902. Dilation tool 900 is then advanced within the mediastinum 966 under endoscopic visualization (tapered tip 904 is transparent to allow endoscopic visualization). A laparoscopic endoscope with an attached CCD camera (not shown) can be used to accomplish endoscopic visualization. Since the pericardium 955 is a thin membrane, visualization of the beating heart through the endoscope underneath a translucent membrane indicates correct positioning of the dilation tool 900 on the anterior surface of the pericardium 955.

Referring to FIG. 21B, the dilation tool 900 is shown advanced to the desired position in the body, and expandable sheath 922 is held in place as inner cannula 908 is retracted through expandable sheath 922 in the direction indicated by arrow 921. Inner cannula 908 has an enlarged region near its tip (not shown) which causes expansion of the sheath 922 during retraction of inner cannula 908. This expansion of sheath 922 dilates the tissue adjacent to the length of expandable sheath 922 to at least the maximal dimension of the enlarged region.

With expandable sheath 922 in place, large diameter instruments can be sequentially inserted through the proximal end of expandable sheath 922 without exerting shear force on the tissue cavity. Expandable sheath 922 accommodates instruments of varying diameters and cross-sections. Thus, leaving expandable sheath 922 in place maintains a dilated cavity to the desired surgical site, facilitating the advancement of the next instrument to be used in the procedure to the correct position within the body.

FIG. 21C illustrates the step of introducing an endoscopic cannula 925 with transparent tapered tip 935, used in the methods of the present invention. Endoscopic cannula 925 is shown about to be inserted into expandable sheath 922, which can expand to accommodate the larger diameter of the endoscopic cannula 925. Endoscopic cannula 925 has an elongated body 918 which includes one or more lumens and an eyepiece or camera mount 915. One of the lumens may be used as an endoscopic lumen to house an endoscope, while another lumen may be used as an access port 909 for housing surgical apparatus, advanced either concurrently or sequentially, as will be described more specifically herein. In order for the endoscopic cannula to be used for introducing a cardiac restraint apparatus according to the present invention, the access port 909 may be approximately 12-15 mm in diameter, at least near the proximal end to facilitate convenient entry of the endoscopic cannula. FIG. 21D shows endoscopic cannula 925 in position inside expandable sheath 922, with tapered tip 935 adjacent to pericardium.

Referring now to FIG. 21E, the pericardium entry instrument 978 (including grasping jaws 975 and rotatable cutting tube 976) is inserted into access port 909 of endoscopic cannula 925 to cut an opening in the pericardium 955 to access the heart. The entry instrument 978 is manipulated to grasp the pericardium 955 with the grasping jaws 975, followed by rotation and distal translation of the cutting tube 976 to cut an opening in the pericardium 955 and permit insertion of endoscopic cannula 925 into the pericardium 955.

FIGS. 21F and 21G illustrate the maneuverability of endoscopic cannula 925 once it is inserted into the pericardium through the opening created by the entry instrument 978. Once inside the pericardium, endoscopic cannula 925 can be swept around the heart 1000 over the anterior and posterior surfaces of the heart 1000 (e.g. from the position shown in FIG. 21F to that shown in 21G) and otherwise maneuvered around the heart 1000 in such a way that all regions of the heart may be accessed. The endoscopic cannula can be maneuvered because of the subxiphoid entry position and the flexibility of soft tissue around the heart, the softness of the tissue allowing the endoscopic cannula to push apart tissue and move around the heart. Thus, all regions of the heart may be accessed without the need for invasively lifting or rotating the heart to access posterior or lateral vessels and structures.

It should be noted that while the above method of accessing the pericardium was described with reference to usage of a dilation tool having an expandable sheath, a dilation tool without an expandable sheath may also be used. In that embodiment, the inner cannula of the dilation tool can be used by itself to dilate a cavity to access the pericardium, and the endoscopic cannula can be inserted into the dilated cavity.

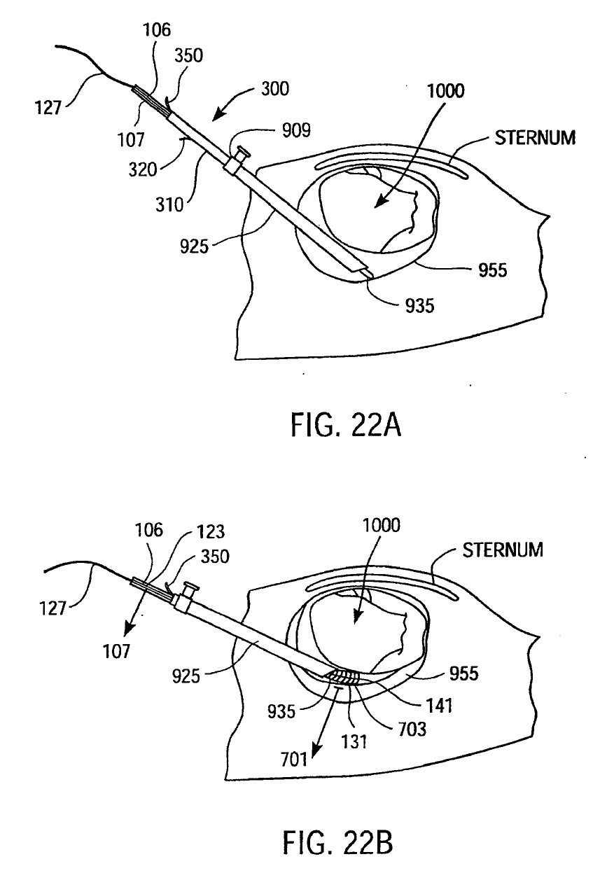

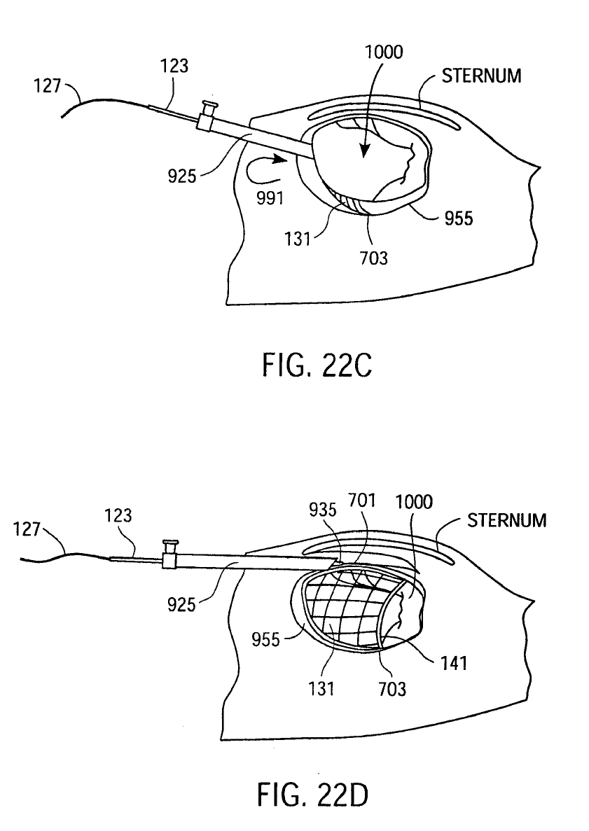

Once the heart is accessed, a cardiac restraint apparatus according to the invention may be introduced and positioned around the heart. FIGS. 22A through 22D illustrate the placement of a cardiac restraint apparatus via subxiphoid incision in accordance with one method embodiment of the present invention. While a subxiphoid approach provides surgical advantages, as discussed above, other entry methods and other approaches for example, trans-xiphoid and thorascopic, may also be used with or without an endoscopic cannula. FIG. 22A illustrates an endoscopic cannula 925 in position on the posterior aspect of the heart 1000 via a subxiphoid approach as previously described, and a sheathed cardiac restraint apparatus 300 according to the invention being inserted into access port 909. Endoscopic cannula 925 also has a second access port, into which a tacking instrument (not shown) is inserted. Alternatively, the tacking instrument is inserted through the lumens defined by each one of guide tubes 106 and 107 in turn instead of through a second access port of endoscopic cannula 925. In this alternative embodiment, guide tubes 106 and 107 each define a lumen sufficiently wide to receive the tacking instrument 701. Guide tubes 106 and 107 are sufficiently long to remain outside of the body while the jacket is placed around the heart.

Next, sheath 320 is removed by pulling pull tab 350 away from the heart, tearing sheath 320 at perforations 310. The removal of sheath 320 frees jacket 131, causing it to unwind from its folded state. The tacking instrument 701 is then used to tack or staple rim 141 to the posterior pericardium near the base of the heart, using guide tubes 106 and 107 to better guide the placement of rim 141 and to hold rim 141 in place in the desired position during tacking. Following placement of tack 703, each guide tube 106 and 107 is detached from rim 141, for example by cutting strand 710 or unraveling knot 726 as illustrated in FIG. 18.

As shown in FIGS. 22C and 22D, endoscopic cannula 925 is then pulled up and over the apex of the heart in the direction of arrow 991, pulling jacket 131 onto the anterior surface of the heart to at least partially enclose the heart with jacket 131. Manipulation of jacket 131 may be aided by enlarging the pericardial opening using a cutting tool as previously described. As shown more clearly in FIG. 17, strand 127 is then pulled away from the heart while knot pusher 123 is pushed against slipknot 670 on rim 141 to tighten jacket 131 around the heart as more clearly illustrated in FIG. 17. Knot pusher 123 is then disengaged from strand 127, and a pair of endoscopic scissors (now shown) are advanced through the cannula to transect the excess tail of strand 127 to conclude the procedure.

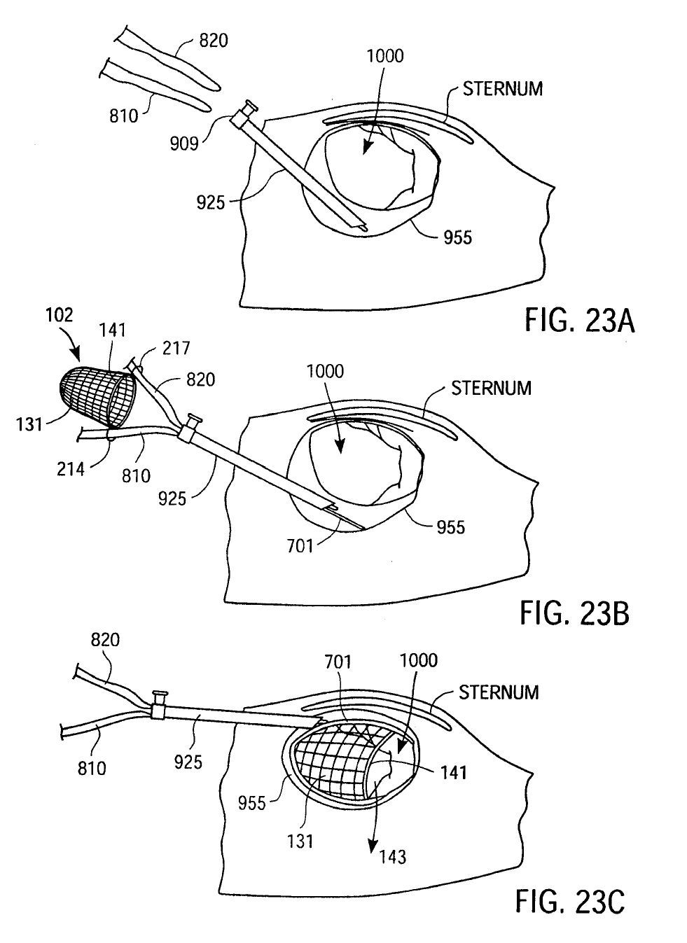

Alternatively, the endoscopic cannula may be advanced to the posterior pericardial space without deployment of the cardiac restraint apparatus, as shown in FIGS. 23A-23C. This alternative method uses an alternative embodiment of a cardiac restraint apparatus, as shown in FIG. 19 and described above in detail. Referring now to FIG. 23A, endoscopic cannula 925 has been positioned within the pericardium as described above. Guide strands 810 and 820 are then introduced into endoscopic cannula 925 via access port 909. Guide strands 810 and 820 can be constructed from any conventional surgical suture material, for example nylon, silk, steel, catgut, and conventional bioabsorbable suture materials such as polymers and copolymers of lactide, glycotide, para-dioxanone and trimethylene carbonate.

Next, tacking instrument 701 is introduced into access port 909 (or alternatively, into a second access port, not shown) as illustrated in FIG. 23B. Guide strands 810 and 820 are tacked to the posterior pericardium using tacking instrument 701. Alternatively, guide strands 810 and 820 can be tied to a tack in the tacking instrument 701 prior to its introduction through access port 909. Guide strands 810 and 820 are then looped through the handles 214 and 217 attached to rim 141 of cardiac restraint apparatus 102, as shown in FIG. 23B. While in this embodiment of the method jacket 131 is in its unsheathed state, jacket 131 may alternatively be sheathed as previously described. Cardiac restraint apparatus 102 is pushed, guided by guide strands 810 and 820, into position posterior to the heart. Guide strands 810 and 820 may be tied extracorporeally, and the knots pushed up to the previously placed tacks, to secure the posterior portion of jacket 131. At this point, if the sheathed configuration of jacket 131 is used, the jacket is unsheathed as previously described, and opening 143 of the jacket 131 is pulled inferiorly around the apex of the heart, then advanced anteriorly into position at the base of the heart as shown in FIG. 23C. The knot pusher at the anterior rim of the jacket is cinched down at the base of the heart 1000 as shown in more detail in FIG. 17 and as previously described, to at least partially enclose the heart. The excess lengths of guide strands 810 and 820 are cut with endoscopic scissors (not shown) to complete the procedure.

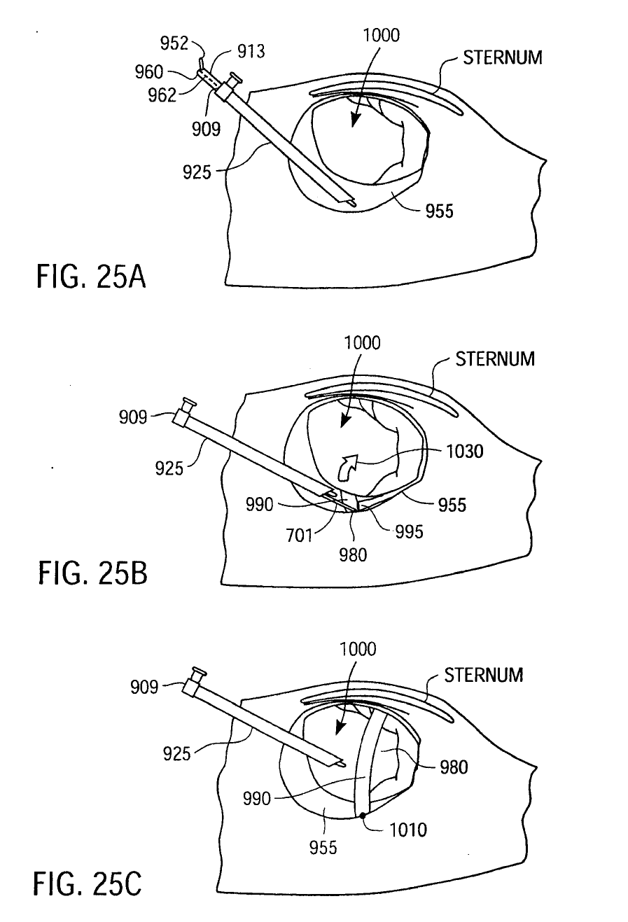

An alternative method embodiment of the invention uses an alternative embodiment of a band-type cardiac restraint apparatus according to this invention, as described above and illustrated in FIGS. 24A-24B. One embodiment of this method, as illustrated in FIGS. 25A-25C, is performed using the subxiphoid access method described above. Referring now to FIG. 25A, endoscopic cannula 925 is introduced into the pericardium as previously described. Cardiac restraint apparatus 960, described above with reference to FIGS. 24A-24B, is then introduced into access port 909 and into pericardium 955 via an opening made in the pericardium in a manner as previously described. The introduction of cardiac restraint apparatus 960 into the pericardium may be optionally facilitated using a rod (not shown) which pushes cardiac restraint apparatus 960 into the pericardium. Sheath 962 is then removed by pulling pull tab 952 which causes the tearing of sheath 962 along perforations 913, releasing elastic band 980 housed within sheath 962.

Next, referring to FIG. 25B, a tacking instrument 701 is introduced into the pericardium through access port 909 to tack elastic band 980 (shown in detail in FIG. 24A) to the posterior pericardium. Preferably, elastic band 980 is tacked to the pericardium at a point located between first portion 990 and second portion 995. Alternatively, elastic band 980 is tacked to the pericardium at any point located between first end 992 and second end 996. Elastic band 980 can also be attached initially to the tack of the tacking instrument 701, prior to introduction of both elastic band 980 and tacking instrument 701 together through access port 909.

Next, as shown in FIGS. 25B and 25C, first portion 990 and second portion 995 of elastic band 980 (more clearly shown in FIG. 24A) are moved from the posterior pericardium to the anterior aspect of the heart, and are tacked to the pericardium overlying the heart, preferably to the anterior aspect of the heart. First portion 990 is moved to the anterior aspect of the heart in the direction of arrow 1030 by advancing a grasping instrument (not shown), for example a clip applier, into the pericardium via endoscopic cannula 925, grasping first portion 990 of elastic band 980 and pulling from the posterior pericardium to the anterior aspect of the heart in the direction of arrow 1030. Optionally, elastic band 980 is configured to receive a grasping instrument, for example by including openings 990 and 991 as shown in FIG. 24A. Second portion 995 is moved in the opposite direction, around the posterior aspect of the heart and over to the anterior aspect of the heart. First portion 990 and second portion 995 are then tacked to the pericardium overlying the heart. The first portion 990 and the second portion 995 can be tacked or clipped together to complete the procedure.

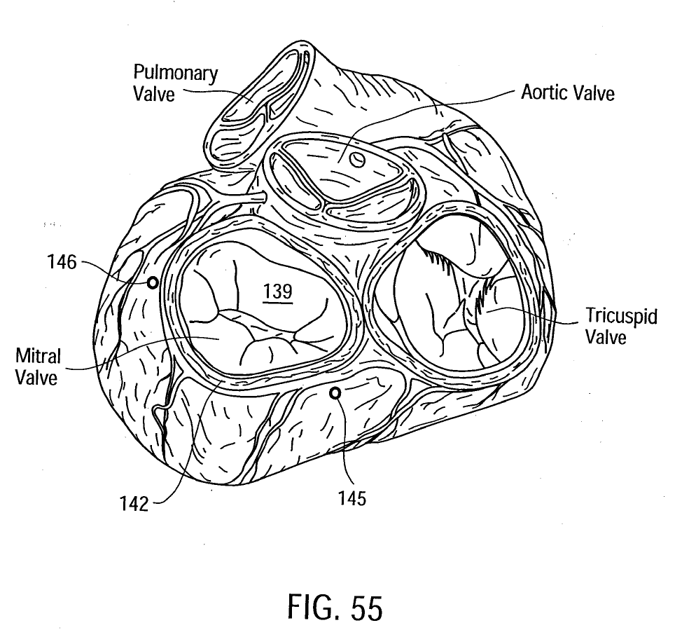

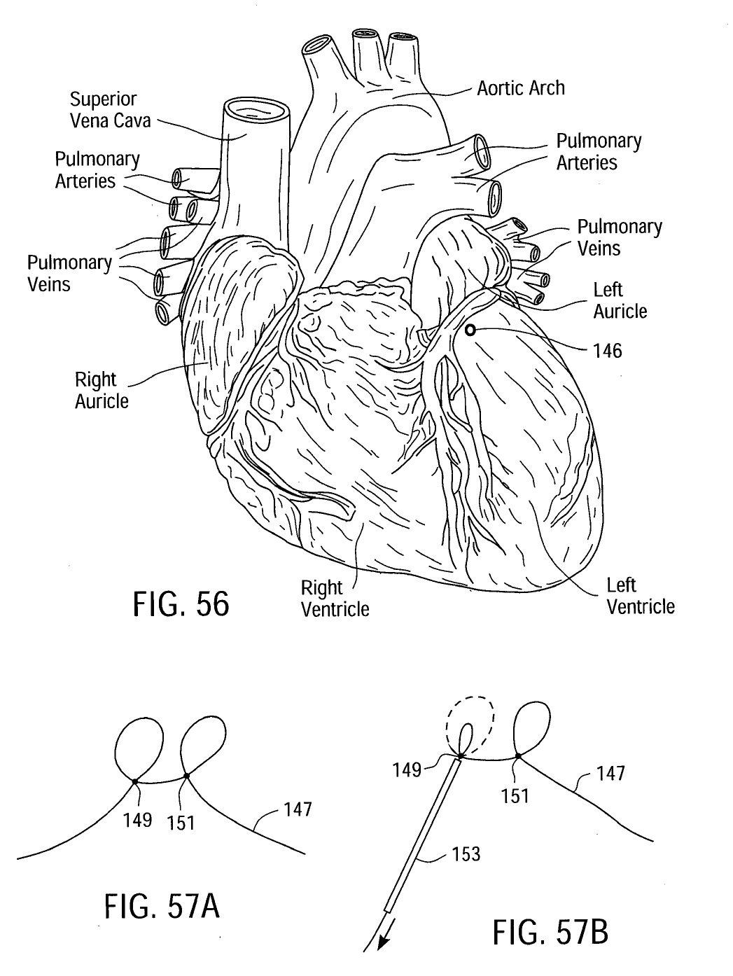

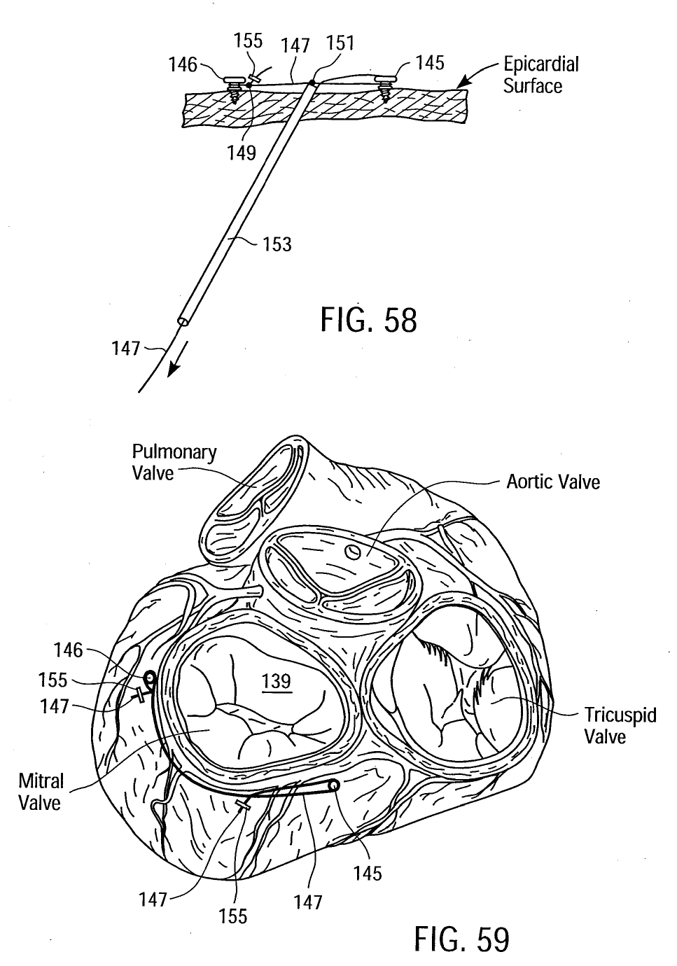

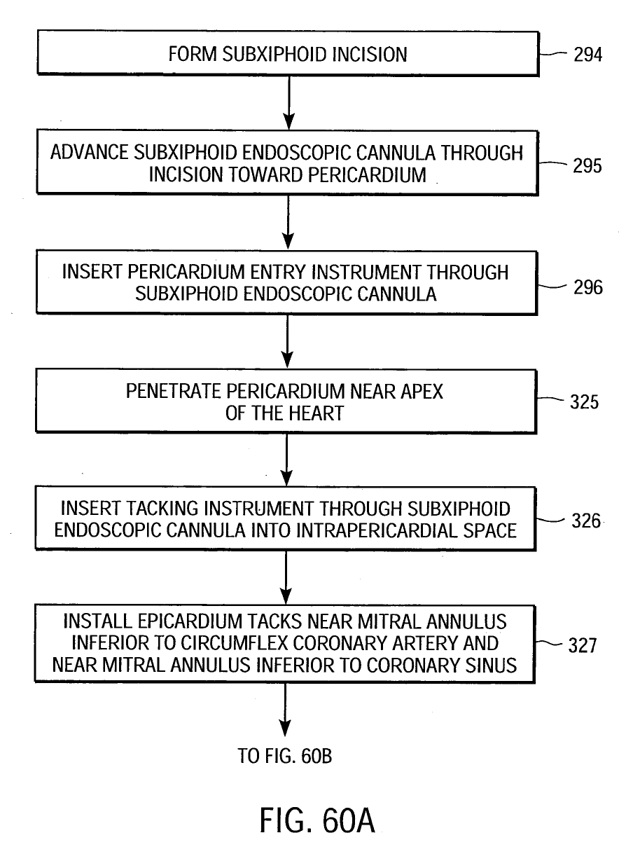

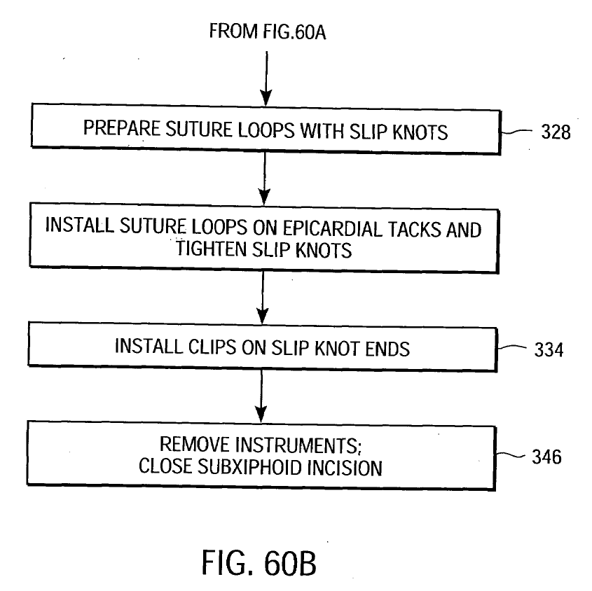

Referring now to the partial or cut-away top view of a human heart illustrated in FIG. 55, there is shown the mitral valve 139 and its annulus 142. In accordance with an embodiment of the present invention, a regurgitant mitral valve may be repaired using subxiphoid access procedures, as previously described herein. Then, one potential tack placement 146 is located inferior to the left circumflex artery in the anterior aspect of the mitral annulus, and another tack placement 145 is located inferior to the coronary sinus in the posterior aspect of the mitral annulus. FIG. 56 shows an anterior view of the heart, showing the tack 146 located inferior to the left circumflex coronary artery. A conventional tack applier (e.g., the PROTACK available from U.S. Surgical) may be introduced through the endoscopic subxiphoid cannula, following the procedure described herein, for example, with reference to steps 294, 295 and 296 of FIG. 60A. Entry through the pericardium is performed by the pericardial entry instrument that is inserted 296 via the operating channel of the endoscopic subxiphoid cannula and the pericardium is penetrated 325, as previously described herein. Following pericardial entry, the pericardial entry instrument is removed, and the tacker shaft is advanced 326 through the operating channel of the endoscopic subxiphoid cannula to apply tacks 327 at the locations 145, 146 shown in FIG. 55. A looped suture or wire 147 is prepared 328 for tensioning of the epicardium by placement 329 onto the tacks, and by applying the desired amount of tension. The tacker is then removed, and an endoscopic grasper is used to apply the looped suture or wire strand 147 to the epicardial tacks 145, 146.

Referring also to FIGS. 57A and 57B, there is shown an embodiment of a tension suture. Two loops are formed in a strand 147 of suture, with a slipknot 149, 151 formed at the base of each loop. The free end of each loop may be threaded through an axially rigid tube 153. The tube 153 functions as a knot pusher to close down on each loop, thereby shortening the distance between the two loops. In use, one loop may be placed on an inserted tack 146 and tightened down. The second loop is placed on the second tack 145 and the tail on the second loop is pulled through the tube 153 to shorten the loop and apply tension between the two tacks. At the desired amount of tension, vascular clips 155 are placed (step 334 of FIG. 60B) at the base of each suture tail to prevent the slipknots 149, 151 from slipping, thereby preserving the tension between the tacks 146, 145.

FIG. 59 shows the anterior tack 146 and a posterior tack 145 in place with a length of suture 147 looped and tightened down on the anterior tack 146. A suture loop extends around the posterior tack 145 and the loop is tightened down and drawn toward the anterior tack 146 to the desired tension. Vascular clips 155 are placed on the suture tails adjacent the respective slipknots 149, 151 and the suture tails are then trimmed short to complete the mitral valvular repair.

In other embodiments of the present invention, a band or belt may be tensioned between anterior and posterior tacks 146, 145 to avoid cutting into the epicardium. Also, additional tacks may be installed in the epicardium at locations about the mitral annulus intermediate the anterior and posterior tacks 146, 145 to facilitate segmented tensioning of sutures or bands or belts from tack to tack about the mitral annulus. Thereafter, the instruments are removed from the body and the subxiphoid incision is closed 346 to complete the procedure.

Therefore, the subxiphoid access to the intrapericardial space facilitates placing epicardial tacks about the annulus of the mitral valve for tensioning the epicardium between tacks to decrease the size of the mitral valve annulus as a repair of a regurgitant valve.

In accordance with another embodiment of the present invention, distention constraints may be externally attached to the heart via subxiphoid access to the heart using an endoscopic cannula.

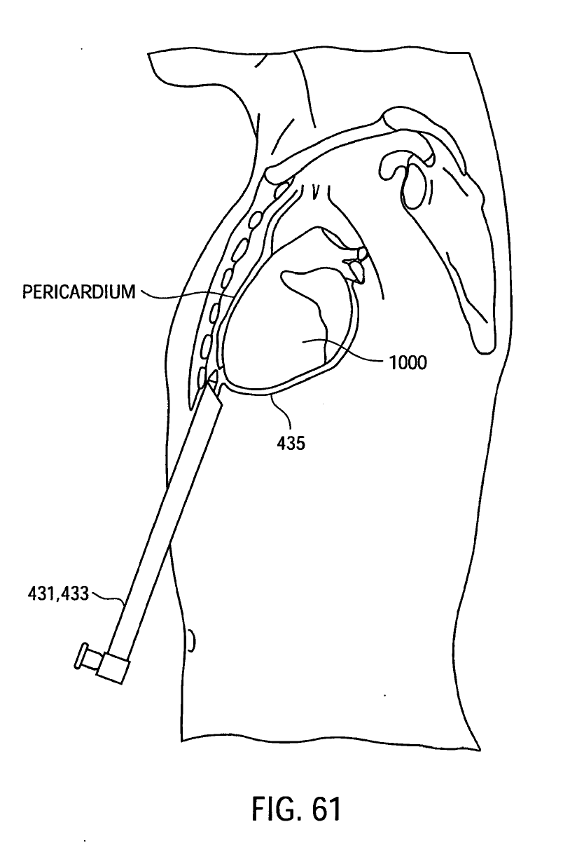

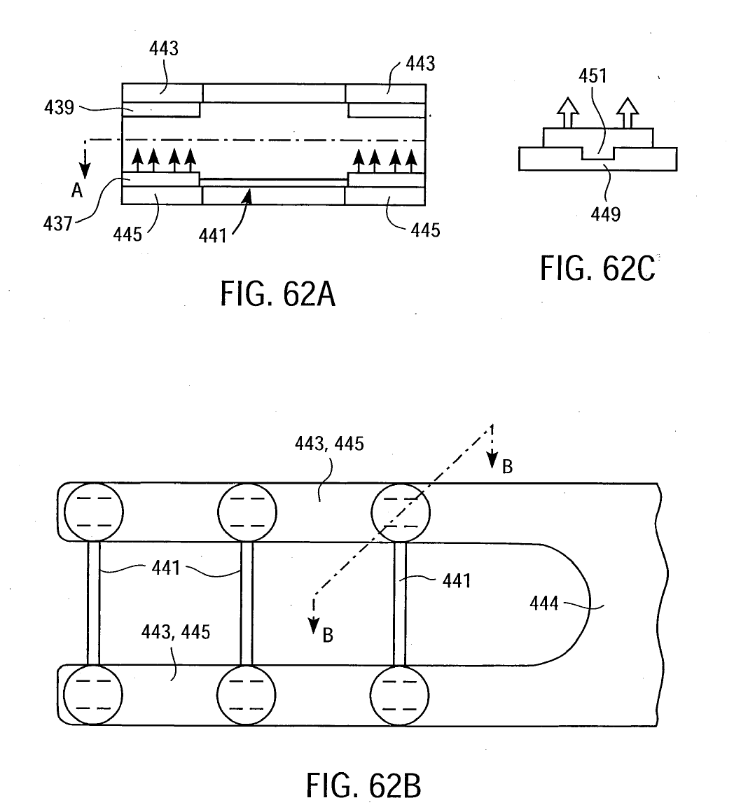

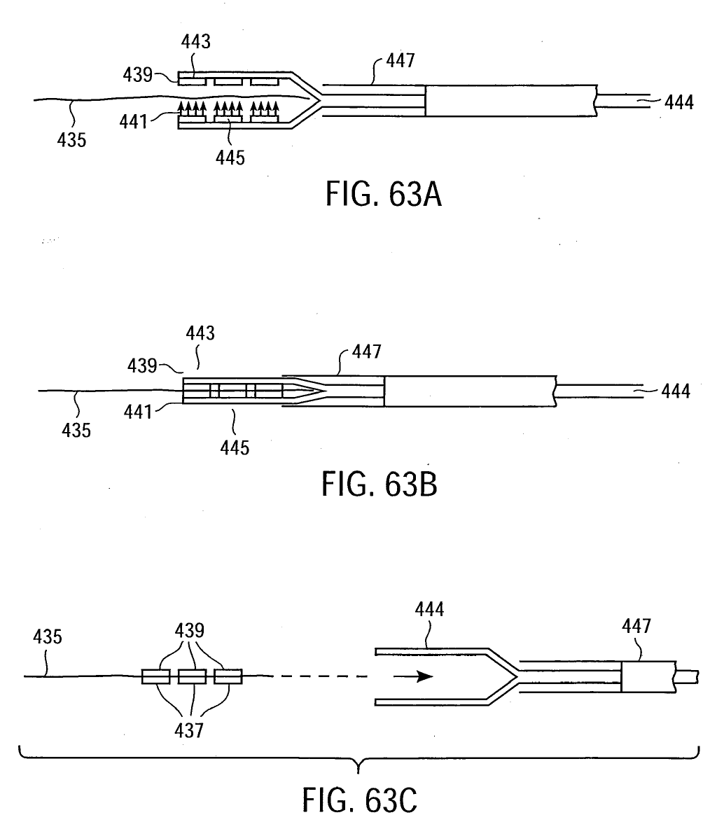

Referring now to FIG. 61, using a subxiphoid endoscopic cannula 431 and a pericardial entry instrument 433, the pericardium 435 is entered on its anterior surface near the apex of the heart 1000. The pericardial entry instrument 433 is removed from the operating channel of the subxiphoid endoscopic cannula 431, and a specialized instrument as later described herein with reference to FIGS. 62A-C, is inserted to apply two sets of tacks 437, 439 each joined by an elastic band 441. The tacks on flange 437 with a plurality of sharpened barbs that pierce through the pericardium 435, and the second flange 439 that is pierced by and locks onto the barbs. Thus, the tack attaches to the pericardium, with a flange placed on each side of the pericardium 435, as shown in FIGS. 63A-C.

The specialized applicator instrument, as shown in FIGS. 62A-C and 63A-C includes two spaced sets of elongated jaws 443, 445 that are spring loaded in an opened position as shown in FIG. 63Aa. An outer frame 447 slides along the shaft of the applicator 444 and the frame is advanced distally to force the jaws into a closed position, as shown in FIG. 63B. The outer frame 447 may contain a tubular portion along most of its length, and a rigid distal portion with open sides. The open sides allows the outer frame to be advanced over the elongated jaws 443, 495 while inserted through the single pericardial incision. The elongated jaws may also contain sets of holes or recesses that accommodate knobs 451 on both the barbed and unbarbed flanges, as shown in FIG. 62B. The holes 449 are a sliding fit with the knobs 451 for holding the flanges in place as they are applied across the pericardium, and releasing the flanges after they are locked on either side of the pericardium as shown in FIG. 63C.

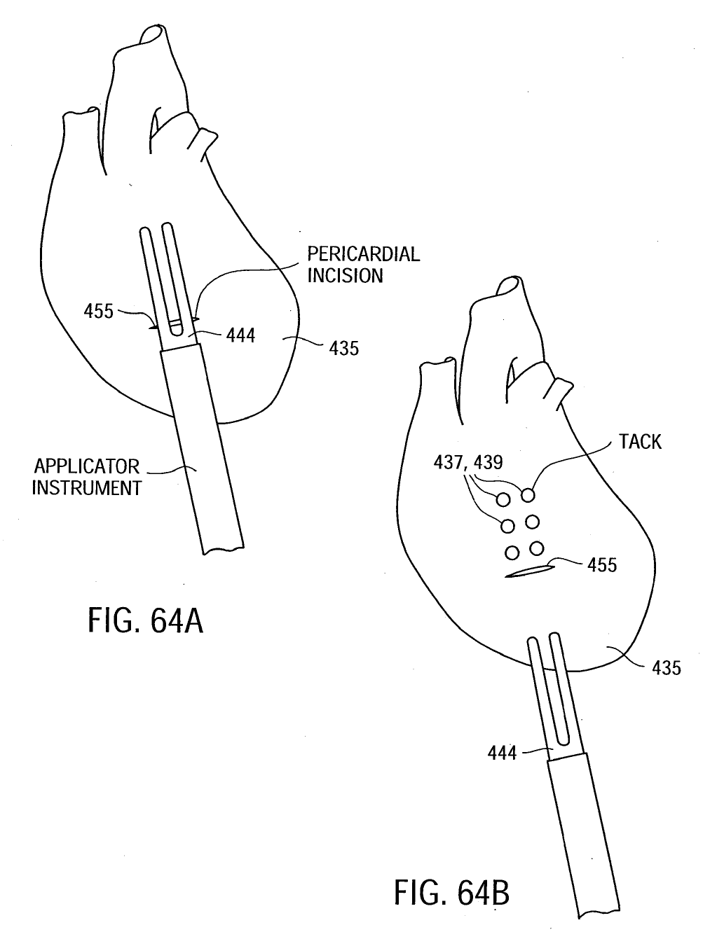

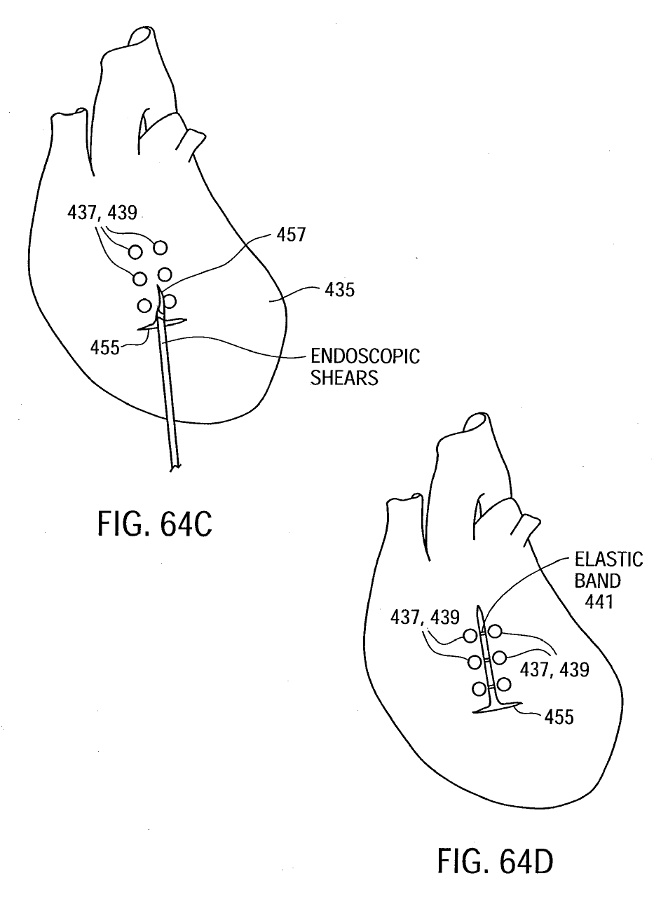

Referring now to FIGS. 64A-D, there is shown the sequence of steps for placement of the cardiac support device including the flanges 437 and elastic band 441, after pericardial incision has been performed. FIG. 64A shows the applicator instrument 444 advanced through an incision 455 on the anterior pericardial surface. The applicator instrument 444 is oriented in a superior-inferior direction in this diagram. Other orientations may be used including, for example, a transverse direction. FIG. 64B shows the tacks 437, 439 in place in the pericardium 435 after placement, and the applicator instrument being removed out of the incision 455. FIG. 64C shows a pair of endoscopic shears incising the pericardium 435 between the two rows of placed tacks 437, 439. FIG. 64D shows the resultant cardiac support, with elastic bands 441 exerting tension between the pairs of opposing tacks 437, 439 in the pericardium. The sequence of steps may be repeated in additional locations to increase the amount of support, or to change the direction of support.

Alternatively, the series of tacks 437, 439 and elastic bands 441 may be placed as illustrated and described without subsequent incision of the pericardium between the rows of tacks. In this configuration, tension is still exerted on the pericardium 435 to generate cardiac support, with the redundant pericardium remaining in place between the rows of tacks.

While surgical procedures have been described above with reference to a subxiphoid approach using an endoscopic cannula, the method embodiments of the invention may use other incisions and approaches such as a subxiphoid incision, a trans-xiphoid incision, and a thorascopic incision, with or without the usage of an endoscopic cannula. In addition, one or more elastic bands of varying widths, preferably three elastic bands each having a width about 1 cm, may also be used. Also, the subxiphoid, or other incisions and approaches as described above, may be used during other surgical procedures on the heart, or other mediastinum organs.

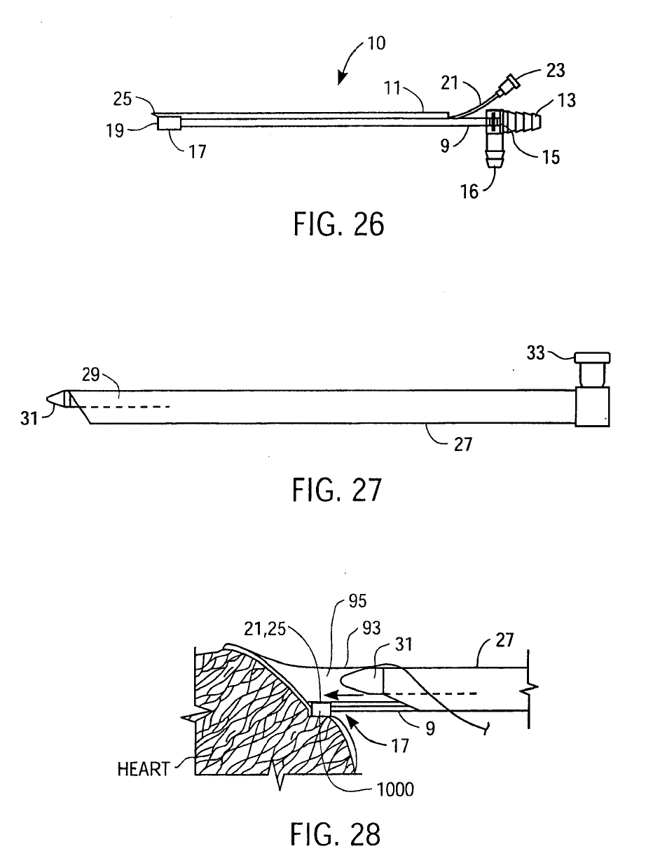

For example, with reference to FIG. 26, there is shown one embodiment of a suction assisted insertion cannula 10 according to the present invention including a closed channel 9 and a superior channel 11 attached to the closed channel for use in surgical procedures on the heart. The closed channel 9 includes a suitable hose connection 13 and a three-way vacuum control valve 15 including an irrigation port 16 at the proximal end, and a suction pod 17 positioned on the distal end. The suction pod 17 includes a porous distal face or suction ports 19 that serves as a vacuum port which can be positioned against the epicardium to facilitate temporary fixation thereto as a result of the reduced air pressure or vacuum supplied to the suction pod 17. The distal end of the superior instrument channel 11 that is attached to the closed channel 9 may thus be held in accurate fixation in alignment with a selected surgical site on the epicardium relative to the suction fixation location of the suction pod 17 on the epicardium. A rounded smooth surface of suction pod 17 may be used to apply gentle pressure on the epicardium to stop bleeding at small puncture sites, or to facilitate injected cells being absorbed without exiting back out of the injection.

The superior channel 11 is sized to accommodate slidable movement therein of a hollow needle 21 that may exhibit lateral flexibility over its length from the needle hub 23 at the proximal end to the sharpened distal end 25. When used to inject cells, the needle 21 may be about 22-25 gauge in diameter and includes an internal bore of sufficient size to facilitate injection of cells without incurring cell damage, or lysis. When used to place pacing or defibrillating leads, the needle 21 may be about 2-2.5 mm in diameter with an internal bore of sufficient size to accommodate a lead of diameter up to approximately 2 mm in diameter.

After the lead is implanted in the heart by the procedure described above, the proximal end is disposed out through the small initial incision in the patient. The proximal end may then be tunneled subcutaneously from the initial incision to an incision in the patient's upper chest where a pacemaker or defibrillator will be located. A small, elongated clamp is passed through the subcutaneous tunnel to grasp the proximal end of the epicardial lead to facilitate pulling the lead through the tunnel for placement and attachment to the pacemaker or defibrillator.

Both the superior channel 11 and the needle 21 may be longitudinally slotted for placing an epicardial lead that may incorporate a large diameter connector. A split sheath can be used around the lead to facilitate advancement and rotation of the lead via the slotted needle. After anchoring such lead in the myocardium, for example by screwing in the distal tip, the slotted needle 21 is rotated to align its slot with the slot in the superior channel 11, thus allowing the lead to be released from the cannula.

The structure according to this embodiment of the invention, as illustrated in FIG. 26, is disposed to slide within the instrument channel in an endoscopic cannula 27, as shown in FIG. 27. This cannula includes an endoscope 29 therein that extends from a tapered transparent tip 31 attached to the distal end, to a viewing port 33 at the proximal end that can be adapted to accommodate a video camera. In this configuration, the structure as illustrated in FIG. 26, or other surgical instrument, may be positioned within the instrument channel in the cannula 27 of FIG. 27 to position the suction pod 17 and sharpened needle tip 25 in alignment with a surgical target on the heart, as illustrated in FIG. 28. The suction pod 17 is temporarily affixed to the epicardium in response to suction applied to the porous face 19 of the suction pod 17 under control of a suction valve 15, and the sharpened tip 25 of the needle 21 may then be advanced to penetrate into the myocardium at an accurately-positioned surgical site, all within the visual field of the endoscope 29 through the transparent tip 31. Following injection, the needle is withdrawn and the suction pod 17 may be rotated or otherwise manipulated to position a surface thereof on the injection site with gentle pressure to allow time for the injected cells to be absorbed and to control any bleeding occurring out of the injection site.

As illustrated in FIGS. 27 and 28, the various channels in the endoscopic cannula 27 and the insertion cannula 10 have specific orientations with respect to each other in order to provide stabilization on the epicardial surface and allow visual control of the injection process. In the endoscopic cannula 27, the instrument channel is positioned below the endoscopic channel and this allows the cannula 27 and the transparent tapered tip 31 on the endoscope 29 to retract the pericardium 93 away from the epicardial surface of the heart at the operative site. This creates a space 95 for contacting the heart below the pericardium 93, as illustrated in FIG. 28. As the cell insertion cannula 9 is advanced forward out of the instrument channel of the endoscopic cannula 27, the suction pod 17 is visualized through the endoscope 29 and transparent tip 31, as the suction pod 17 is placed on the epicardial surface of the heart. At a selected site on the heart, for example, at the site of an old myocardial infarct, the suction is activated to attach the pod 17 to the heart. The configuration of the instrument channel of the cell insertion cannula 10 on top of the suction channel 9 allows the needle 21 to be visible as soon as it exits from the instrument channel, and remain visible within the visual field of the endoscope along the entire path of travel of the needle 21 from the insertion cannula 10 to its insertion into the myocardium. Continuous visualization of the needle 21 in this manner helps to prevent inadvertent puncture of a coronary vessel.

The configuration of the suction pod 17 and the needle 21 on the insertion cannula 10 also facilitates delivery of substances or devices in an orientation perpendicular to the epicardial surface. For placement of pacing or defibrillation electrical leads, it is particularly desirable to have the leads enter the myocardium in an orientation that is generally perpendicular to the epicardial surface for secure anchoring in the myocardium. Generally, the insertion cannula 10 is advanced through the endoscopic cannula 27 and approaches the epicardial surface of the heart at a tangential angle. Accordingly, the insertion cannula 10 is configured to facilitate deforming the epicardial surface in order to achieve perpendicular entry of the needle 21 into the myocardium, as illustrated in FIG. 28. The suction pod 17 of the insertion cannula 10 temporarily attaches to the epicardial surface upon application of vacuum under control of the valve 15. Downward pressure can be exerted on the epicardial surface via the substantially rigid insertion cannula 10. The pliable myocardium thus deforms to create a surface ledge on the heart 1000 distal to the suction pod 17 oriented perpendicular to the axis of the superior instrument channel 11 of the insertion cannula 10, as illustrated in FIG. 28. As the needle 21 is advanced, it enters the myocardium generally perpendicularly to the epicardial surface as thus deformed for desirable lead placement or cell injection.

Referring now to FIG. 28, it should be noted that the insertion cannula 10 is sized to fit in slidable orientation within the working channel of about 5-7 mm diameter in the endoscopic cannula 27. The outer dimensions of the suction pod 17 are less than 5-7 mm diameter and is configured on the distal end of the closed channel 9 not to obstruct the forward movement of the needle 21 past the distal surface 19 of the suction pod 17.

A sharpened distal end 25 of a needle 21 may include a relatively short, sharpened bevel of length approximately 2-3 times the diameter of the needle. Such short bevel length of the needle assures that cells are injected within the myocardium, and that part of the needle bevel does not extend into a heart chamber, with resultant intracardiac cell delivery.

A needle stop may be built into the needle 21. Such a stop may simply be the hub 23 of the needle, and the needle 21 may be sufficiently limited in length that only a specific length of needle, for example, 1 cm, may extend out of the instrument channel of the cell insertion cannula 10 when the needle hub 23 abuts against the proximal face of the instrument channel 11. Alternatively, a distal visual and tactile marker such as a ring or collar of extended diameter may provide generally more precise guide to depth of needle penetration under conditions of different angles of possible needle insertion with respect to the epicardial surface. With an extremely shallow angle of entry, a needle of short length may not enter the heart at all. In use, the transparent tip 31 and the suction pod 17 of the assembled cell injection device may be manipulated to reshape a localized portion of the epicardial surface of the heart to allow perpendicular entry of the needle into the myocardium, as illustrated in FIG. 28. With the suction pod 17 activated, gentle manipulation of the insertion cannula allows adjustment of the needle entry angle while maintaining temporary vacuum-assisted attachment to the epicardial surface, as shown in FIG. 28.

The insertion device may also inject substances other than cells. Angiogenic agents such as vascular endothelial growth factor (VEGF) may be injected into myocardial scar tissue in an attempt to stimulate neovascularization, or growth of new blood vessels into the area. Insertion of the needle itself into myocardial tissue may be therapeutic as a form of transmyocardial revascularization (TMR). It is believed that needle insertion injury may stimulate angiogenesis, or growth of new vessels into a devascularized portion of the heart. The cell insertion cannula thus promotes accurate placement of a needle 21 into myocardium under continuous visualization. When combined with the endoscopic cannula, the needle placement may be accomplished through a small, 2 cm subxiphoid skin incision.

The illustrated embodiment of the insertion cannula includes a substantially rigid cannula containing a closed channel 9 ending in a distal suction pod 17, and a superior instrument channel 11 ending immediately proximal to the suction pod 17 on the closed channel 9. In operation, a long needle is advanced through the instrument channel 11. The needle 21 contains a marker of a type as previously described positioned immediately proximal to its beveled tip 25 that serves as a visual or other sensory indicator of the depth of needle insertion. The marker may be a segment of expanded diameter to provide tactile feedback upon insertion into myocardial tissue. For example, a gold-colored metallic sleeve may be welded or soldered onto the needle 21 to provide both visual and tactile feedback of the depth of penetration of the needle tip into the myocardium. The marker may alternatively include a series of rings etched in the needle or a band etched or sandblasted in the same area. A three-way valve 15 on the cannula 9 allows suction in the pod 17 to be turned on or off, and allows irrigation fluid such as saline to be injected through the suction pod 17 while suction is turned off.

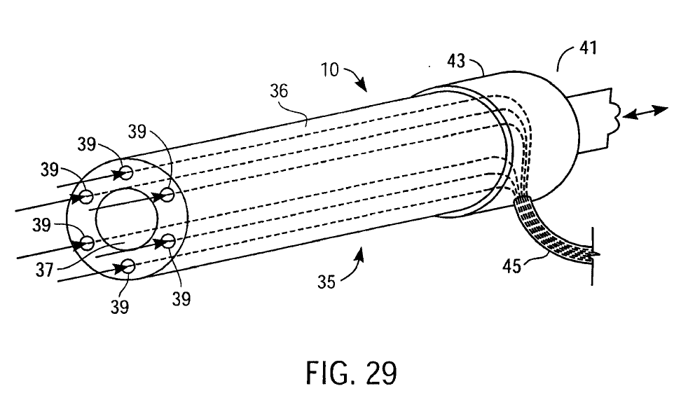

Referring now to FIG. 29, there is shown a perspective view of another embodiment of an insertion cannula 35 similar to insertion cannula 10 described above, including an elongated body 36 having a central bore 37 therethrough to serve as an instrument channel, and including one or more eccentric channels 39 that serve as suction conduits. The central bore may be sized to slidably support surgical instruments 41 therein such as tissue cutters and dissectors, electrocoagulators, injection needles, and the like. For example, surgical instrument 41 may be an energy-supplying ablation probe for epicardial ablation of myocardial tissue in the treatment of cardiac arrhythmia such as atrial flutter or atrial fibrillation. Such an ablation probe 41 may use radio frequency, microwave energy, optical laser energy, ultrasonic energy, or the like, to ablate myocardial tissue for arrhythmia correction. The suction pod 17 attaches to the epicardial surface while suction is turned on at valve 15 to facilitate advancing an ablation probe 41 through the cannula 35 into contact with the heart at the desired site under direct endoscopic visualization for precise myocardial ablation.

The left atrial appendage is frequently the site or source of thromboemboli (blood clots) that break away from the interior of the left atrial appendage and cause a stroke or other impairment of a patient. An ablation probe 41 can be used in the cannula 35 to shrink and close off the appendage to prevent thromboemboli from escaping.

In a similar procedure, a suture loop or clip can be placed through the cannula 35 and applied tightly around the atrial appendage to choke off the appendage.

The suction channels 39 in the cannula 35 of FIG. 29 may form a suction attachment surface at the distal end of the cannula 35, or may be disposed in fluid communication with a suitable suction pod with a porous distal face and with a central opening in alignment with the central bore 37. The suction-attaching distal face provides an opposite reaction force against a tool that exerts a pushing force such as a needle, screw-in lead tip, or other device deployed through the central bore 37 of the cannula 35. The proximal ends of the eccentric channels 39 are connected via a manifold or fluid-coupling collar 43 to a vacuum line 45. Alternatively, a single channel 39 may communicate with an annular recess or groove disposed concentrically about the central bore 37 within the distal end to serve as a suction-assisted attachment surface.

In this configuration, an injection needle 21 slidably disposed within the central bore 37 may be extended beyond the distal end of the cannula 35, within the visual field of an endoscope, in order to orient the needle in alignment with a surgical target site on the pericardium prior to positioning the distal end of the cannula on the pericardium and supplying suction thereto to temporarily affix the cannula 35 in such position. A cannula 35 formed of transparent bioinert material such as polycarbonate polymer facilitates visual alignment of the cannula 35 and the central bore 37 thereof with a surgical site, without requiring initial extension of a surgical instrument, such as a cell-injection needle, forward of the distal end within the visual field of an endoscope. In an alternative embodiment, the central lumen or bore 37 may serve as a suction lumen with multiple injection needles disposed in the outer lumens 39.

The endoscopic cannula and pericardial entry instrument may also be applied from a thoracotomy incision to gain access to the heart. A 2 cm incision is performed in an intercostal space in either the left or the right chest. Ideally, the incision is made between the midclavicular line and the anterior to mid axillary line. The incision is extended through the intercostal muscles and the pleura, until the pleural cavity is entered. The endoscopic cannula is then inserted into the pleural cavity and advanced to the desired area of entry on the contour of the heart, visualized within the pleural cavity. The pericardial entry instrument and procedure as previously described herein are used to grasp the pleura, and a concentric tubular blade cuts a hole in the pleura, exposing the pericardium underneath. The pericardium is then grasped by the pericardial entry instrument, and the tubular blade is used to cut a hole in the pericardium, allowing access to the heart. The transparent tapered tip 31 of the endoscopic cannula 29 aids in pleural and pericardial entry by retracting lung and pleural tissue that may impede visualization of the pericardial entry site. Once the pericardium is entered, the endoscopic cannula 29 may be moved around to visualize anterior and posterior epicardial surfaces.

The surgical apparatus and methods of the present invention provide careful placement of an injection needle or other surgical instrument on the surface of a beating heart by temporarily affixing the distal end of a guiding cannula at a selected position on the heart in response to suction applied to a suction port at the distal end. The guiding cannula can be positioned through a working cavity formed in tissue between the heart and a subxiphoid or other entry incision to minimize trauma and greatly facilitate surgical treatment of a beating heart. Such treatments and procedures may include needle punctures of the myocardium, or injections therein of undifferentiated satellite cells, or other materials, to promote vascularization or tissue reconstruction, for example, at the site of a previous infarct. Such treatments and procedures may also include placing of pacing or defibrillating leads into the myocardium. Such treatments and procedures may further include positioning and manipulation of an ablation probe to ablate myocardial tissue and correct cardiac arrhythmias.

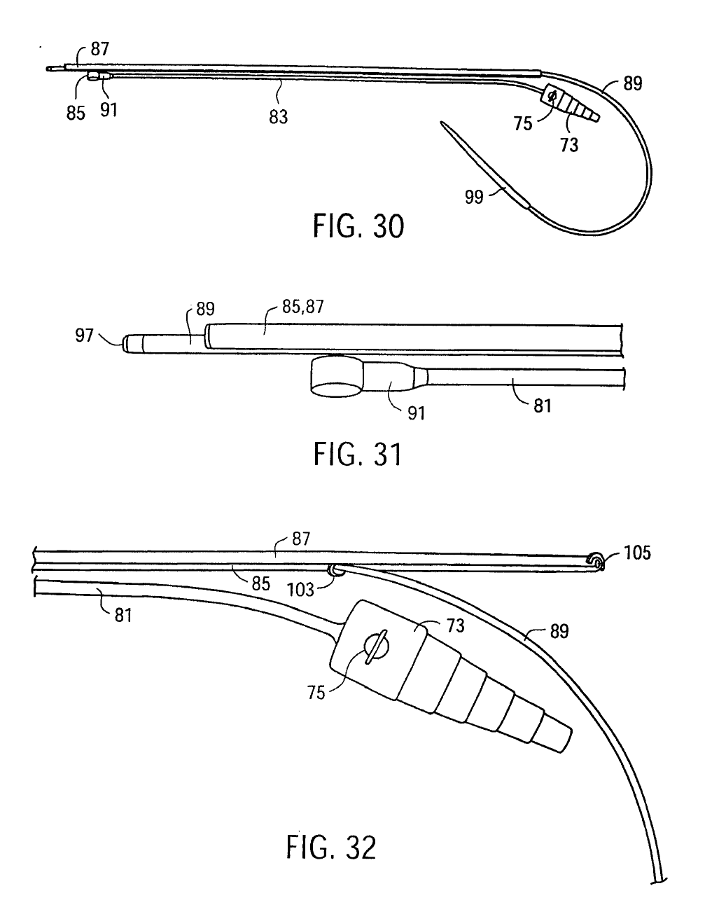

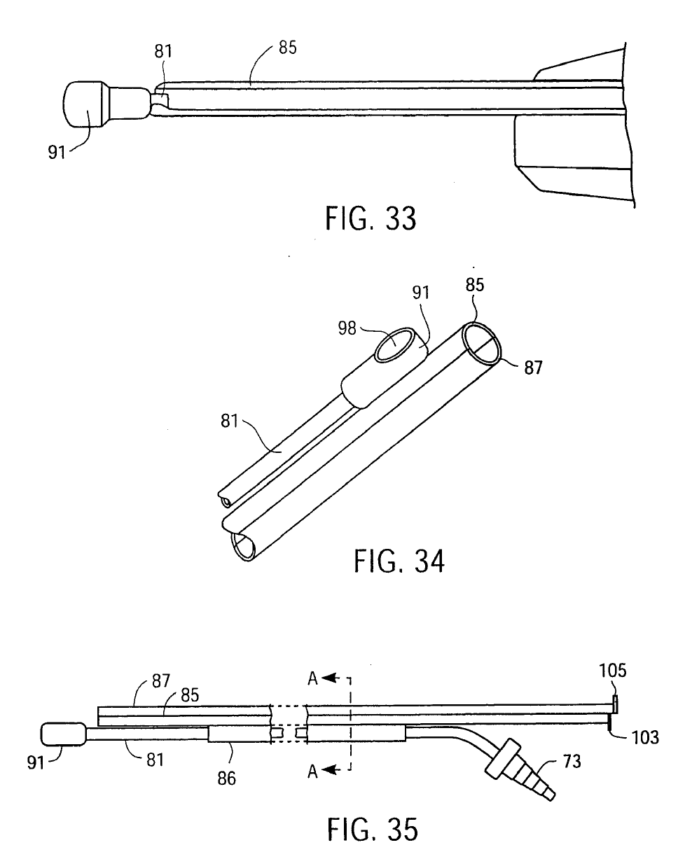

Referring now to the plan view of FIG. 30, there is shown an assembly of suction tube 81 slidably disposed within a guide tube 83 to which is mounted a lower, slotted segment 85 of a guide channel. An upper, slotted segment 87 of the guide channel is slidably rotatably received within the lower slotted segment 85 and a cardiac pacing or defibrillating lead 89 is housed within the guide channel that is configured in the one orientation of the upper and lower segments as a closed guide channel. Another configuration of the upper and lower segments of the guide channel, as later described herein, forms an open channel or slot, as shown in FIG. 33 later described herein, for convenient release of the cardiac lead 89.

The suction tube includes a suction pod 91 at the distal end thereof and a suction-line connection fitting 73 at the proximal end for convenient hose or tubing attachment to a source of vacuum. Optionally, the connection fitting 73 may include a suction control valve 75 for adjusting the suction attachments of the suction pod to the epicardium of a patient's heart.

The cardiac pacing or defibrillating lead 89 is slidably and rotatably housed within the guide channel 85, 87 in the closed configuration, and includes a helical or screw-in electrode 97 attached to the distal end of the cardiac lead 89, as illustrated in FIG. 31. This greatly facilitates electrically connecting and mechanically anchoring the electrode in the myocardium of a patient's beating heart by rotating and advancing the proximal end 99 of the cardiac lead 89 within the guide channel 85, 87. For this purpose, the cardiac lead 89 exhibits high torsional and compressional rigidity and high lateral flexibility so that the electrode 97 may be accurately manipulated into screw-like attachment to the myocardium via manual manipulation of the proximal end 99 of the cardiac lead 89. Such cardiac lead 89 may include braided multiple strands of wire coated with a layer of insulating material such as Teflon, or the like. The accuracy of placement of the screw-in electrode 97 in the myocardium of a patient's beating heart is significantly enhanced by temporary suction attachment of the suction pod 91 to the pericardium or exposed myocardium. The suction pod 91 includes a suction port 98 that may be disposed in lateral or skewed orientation relative to the elongated axis of the suction tube 81. This facilitates the temporary suction attachment while the electrode 97 at the distal end of the cardiac lead 89 that is slidably guided within the guide channel 85, 87 (which is disposed in substantially fixed axial orientation relative to the suction pod 91 and vacuum tube 81) is being anchored into the myocardium.

After the electrode 97 on the distal end of the cardiac lead 89 is anchored into the myocardium of a patient's beating heart, the guide channel that houses the cardiac lead 89 may be re-configured into the alternate configuration including a slot along the length of the guide channel, as illustrated in FIG. 33, from which the cardiac lead 89 may be easily extracted or released. This open slot configuration may be achieved by sliding the upper segment 87 proximally along the lower segment 85, as illustrated in FIG. 32, or by rotating the upper segment 87 within the lower segment 85, as illustrated in FIG. 34. In this way, a longitudinal slot or groove is opened along the entire length of the guide channel that is wide enough to extract the cardiac lead 89 therethrough. This is particularly important for anchoring a cardiac lead 89 of about 2 mm diameter that includes a proximal connector 99 which is too large to pass through a guide channel 85, 87 of reasonable interior dimension.

As illustrated in the perspective view of FIG. 34, the suction port 98 in suction pod 91 is oriented in skewed, typically perpendicular, orientation relative to the elongated axis of the guide channel that is formed by the upper and lower segments 87, 85. This facilitates establishing temporary vacuum-assisted attachment of the suction pod 91 to the epicardium, or to myocardium exposed via the entry under the pericardium, that can then be depressed or otherwise distorted by manual application of axial or lateral force at the proximal end of the instrument in order to position the electrode 97 at the proper location and angle for anchoring in the myocardium of the patient's beating heart.

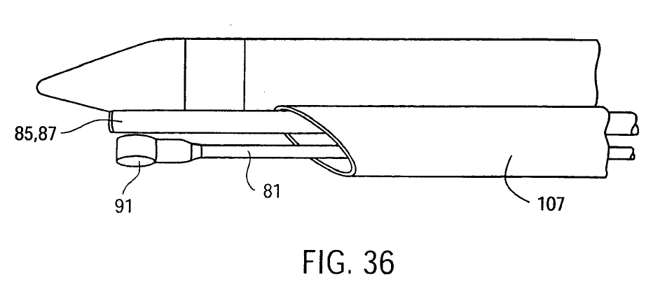

Referring now to the partial plan view of FIG. 35, there is shown a non-round guide tube 96 that is attached to the lower segment 85 of the guide channel and that slidably supports therein the suction tube 81 of corresponding non-round cross section. In this way, the guide channel formed by segments 85, 87 is retained in substantially parallel axial alignment with the suction tube 81 as the suction pod 91 and the distal end of the guide channel are relatively slidably positioned near and against the epicardium of a patient's heart. In addition, the assembly of guide tube 96 and suction tube 81 and guide channel 85, 87 may all be disposed within an endoscopic cannula 107 having a distal end disposed to facilitate endoscopic viewing of the suction pod 91 and distal end of the guide channel 85, 87, as shown in FIG. 36. Also, the upper and lower segment 85, 87 of the guide channel may include stepped flanges 103, 106 at the proximal ends thereof to facilitate positive orientation of the upper and lower segments 85, 87 in the closed configuration until the upper segment 87 is slid proximally, or slid proximally and rotated, relative to the lower segment 85 in order to re-configure the guide channel in the alternate configuration of an open elongated slot along the entire length thereof. The upper 87 segment can be rotated in the lower segment 85 from the closed configuration in order to align the respective elongated slots 88, 108 sufficiently to release a cardiac lead 89 from within the guide channel.

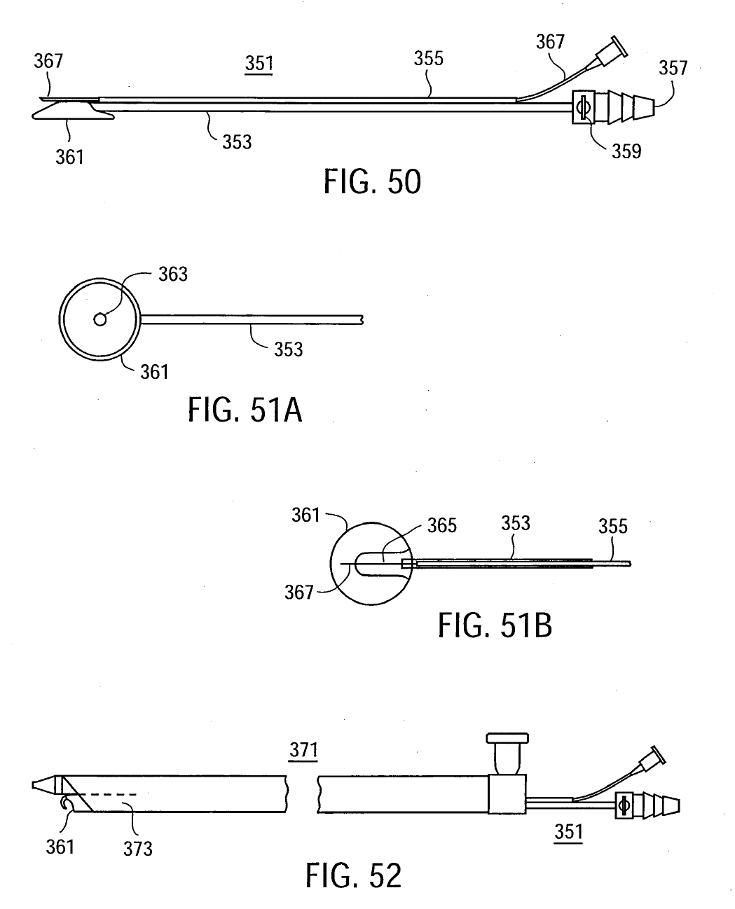

Referring now to FIG. 50, there is shown a plan view of another embodiment of a vacuum-assisted suction cannula 351 according to the present invention that includes an inferior suction channel 353 and a superior instrument channel 355 aligned therewith substantially over the entire length of the inferior suction channel 353 between distal and proximal ends thereof. The cannula 351 may be flexible, steerable, articulatable, rigid, twistable or have other desirable mechanical characteristics that facilitate manipulation of an ablation probe, as previously described herein. Specifically, the proximal end of the inferior suction channel 353 includes a hose connection 357 for attachment to a vacuum supply, and a manually-controllable suction valve 359 for selectively altering the pressure differential relative to ambient pressure within the suction channel 353.

The distal end of the suction channel 353 includes at least one flexible, resilient suction cup 361 disposed with a central axis thereof substantially orthogonal to the elongated axis of the suction channel 353. In an alternative embodiment, a suction cup 361 may be flexibly attached to the suction channel 353 for positioning and manipulating at selected angular orientations relative to an elongated axis of the cannula 351.

As illustrated in the bottom view of FIG. 51A, the interior recess of the suction cup 361 includes a suction port 363 in fluid communication with the suction channel 353. Also, as shown in the top view of FIG. 51B, the suction cup 361 may attach via a resilient, press-fit flange 365 or resilient conduit onto the distal end of the inferior suction channel 353. The superior instrument channel 355 is illustrated in FIG. 51B as overlaying the flange 365, for example, to slidably support therein an ablation probe, for example, as described herein or an elongated, flexible needle 367 capable of delivering medications, injecting undifferentiated cells, installing electrical conductors, or the like, in a bodily organ such as the heart. Alternatively, the suction cup 361 may be attached via flexible coupling to the suction channel 353 and in fluid communication therewith to facilitate temporary suction attachment of the instrument to an organ such as the heart at any convenient angle of approach.

Referring now to FIG. 52, there is shown a plan view of the assembled endoscopic cannula 371 and suction cannula 351 of FIG. 50, with the suction cannula 351 slidably disposed within the instrument channel 373 of the endoscopic cannula. Specifically, the resilient suction cup 361 may be curled or wrapped about an axis aligned with the axis of the inferior suction channel 353 for slidable passage through the instrument channel 373. The resilient suction cup 361, once extended distally outside the instrument channel, resiliently expands to the undeformed cup shape to provide a large contact area of vacuum-assisted contact, for example, with the pericardium in or about the apex area of a patient's heart. The suction cup 361 may be re-coiled or re-wrapped about the axis of the inferior suction channel 353 for return to the instrument channel 373 or the subxiphoid endoscopic cannula in response to withdrawal or retraction of the inferior and superior channels 353, 355 back through the instrument channels 353, 355 back through the instrument channel 373, and in response to the peripheral edges of the suction cup 361 coming into contact with the angled distal edge of the instrument channel 373.

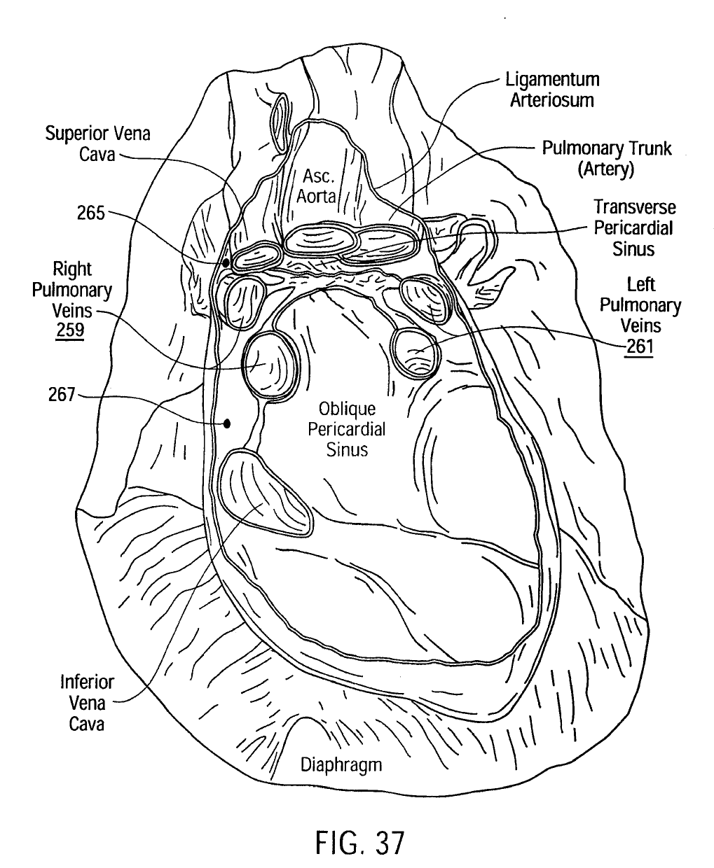

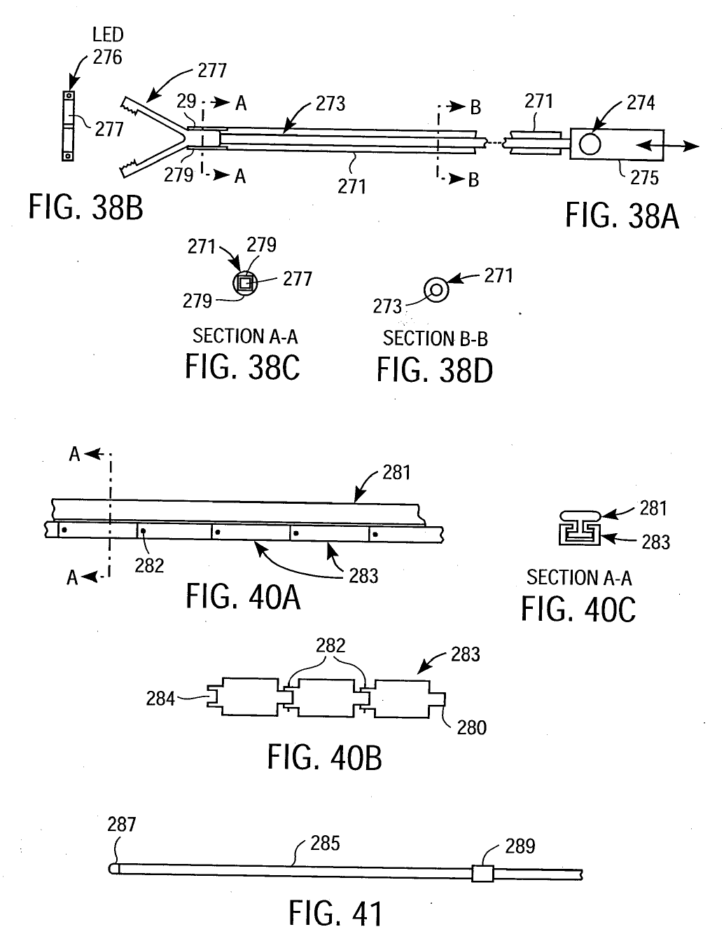

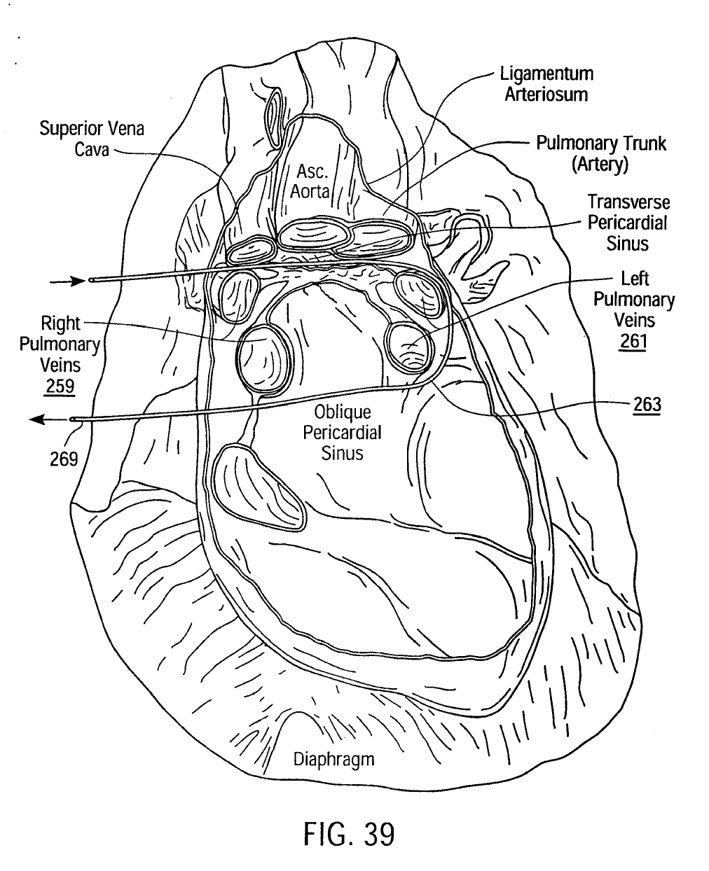

In accordance with another embodiment of the present invention, a treatment for chronic atrial fibillation includes ablating cardiac tissue encircling the pulmonary veins 259, 261. Such treatment may be accomplished in accordance with the present invention using an endoscopic cannula or probe via subxiphoid and thoracotomy access. Referring to FIG. 37, there is shown an anterior view of the interior of the pericardial sac (with the heart removed) that indicates the spatial orientations of various vessels including the right and left pulmonary veins 259, 261. Specifically, an ablation probe, as later described herein, or a tubular sheath therefor may be initially threaded around the pulmonary veins along a path 263 as indicated in FIG. 39, and the ablation probe may be subsequently advanced into position along the path 263 through the tubular sheath. In one embodiment, an endoscopic cannula enters the pericardium from a subxiphoid incision along a dissected channel in order to visualize the superior vena cava and place an illuminating clip, as illustrated in FIGS. 38A-D, at a location 265 on the pericardium adjacent the superior vena cava. Of course, other detectable energy sources or elements may also be positioned in an end effector such as a scissor-like structure including blades or jaws or other effector elements, or in a distal-end illuminator in place of an illuminated clip, using detectable sources such as infrared, ultrasound, fluoroscopy, and the like. The endoscopic cannula is then also used to visualize the inferior vena cava and an illuminated clip or other detectable energy source is then also attached to the pericardium at a location 267 adjacent the inferior vena cava. Once in a desired position, the jaws of the clip 277 are closed on pericardial tissue, for example, by sliding the shaft 273 and manual manipulator 257 backward relative to the tubular body 271. The dimensions of the illuminated clip 277, including the tubular body 271 and the manipulator 275, are smaller than the cross sectional dimensions of the instrument channel of the subxiphoid endoscopic cannula, which can therefore be removed from the body while leaving the illuminated clip in place. Similarly, if a fiber optic cable is attached to the clip, the smaller dimensions of the fiber optic cable and clip allow removal of the subxiphoid endoscopic cannula while leaving the clip in place to be illuminated by subsequent attachment of a light source to the proximal end of the fiber optic cable. The endoscopic cannula can then be removed from the mediastinum following attachment of the clip for insertion of the endoscopic cannula (or insertion by another endoscopic cannula) into the right pleural cavity through a small thoracotomy incision. The light from each clip, or other detectable energy source, as discussed above, at the locations 265, 267 aids in guiding a pericardial entry instrument, and in guiding an endoscopic cannula with a transparent tapered tip during blunt tissue dissection under the superior and inferior vena cava along the path 263, 269 within the intrapericardial space, as shown in FIG. 39.

Referring again to the views in FIGS. 38A-D of an illuminated clip, there is shown an elongated tubular body 271 which can be rigid or flexible or malleable or otherwise adjustable articulateable or steerable. The tubular body 271 includes an inner lumen extending therethrough between distal and proximal ends thereof. An inner shaft 273, which can have the physical characteristics described above for body 271, is slidable within the lumen in the tubular body 271, and includes a manual manipulator 275 attached to the proximal end and a clip 277 with resilient jaws or other suitable attachment mechanism such as barbs disposed in attached or detachable configuration to the distal end of the shaft 273. A square, or other non-rotational shape of the tubular body 271, as shown in the sectional view of FIG. 38B, retains a mating shape of clip 277 in proper alignments with lots 279 that are oriented to facilitate expansion of the jaws of clip 277 toward an open configuration. As the shaft 273 and manual manipulator 275 and clip 277 slide forward relative to tubular body 271, the jaws of the clip resiliently extend into the open configuration, as shown. One or more of the jaws of clip 277 may include a light-emitting diode (LED) 276 as a light source for transluminating the surgical site through the pericardium to which the jaws may attach. Of course, other light sources such as point-to-point cabling of optical fibers from a remote light source to the jaws of clip 277 may also be used, and a switch 274 or other controller may be housed in the manual manipulator 275 for convenient control of light made selectively available at the clip 277 that is positioned, for example, in the manner as previously described.

Referring now to FIGS. 40A-C, there is shown an embodiment of a tissue-ablating instrument or probe according to one embodiment of the present invention that can be inserted in the dissected channel through tissue (or in the insertion tube therefor) along the path 263, 269 within the intrapericardial space. Specifically, the tissue-ablating instrument includes a flexible or steerable or articulatable guide or sheath 281 and an articulated backbone 283 attached to the sheath 281 along a selected length of the instrument. In one embodiment, the backbone 283 includes a plurality of successive segments 283 that are each pinned 282 or hinged together in iterative tongue 284 and groove 280 array, as illustrated in FIGS. 40A, 40B, 40C, to provide lateral flexibility with torsional and longitudinal rigidity. Alternatively, a braided sheath 375, as illustrated in FIG. 53, may include non-round cross section to facilitate retaining an ablation probe of similar non-round mating shape in proper axially angular orientation during slidable positioning along the length of the sheath. In other embodiments, the backbone may provide telescoping control of length and/or torsional control in conventional manner to facilitate twisting all or part of the length thereof into conformal orientation against cardiac tissue. Also, these forms of control over the mechanical characteristics of the supporting backbone facilitate the manipulation of the ablating instrument through the anatomy. This assures that the ablating instrument can be positioned and retained in continuous orientation toward or against cardiac tissue along the path 263, 269 under the pericardium for proper application of tissue-ablating energy only to the cardiac tissue. For example, the distal portions of the ablation probes contain a section that emits tissue-ablating energy. The supplied energy at various wavelengths heats cardiac tissue. Radio-frequency energy may be monopolar, that is, the current supplied via the probe travels through the patient's body to a cutaneous grounding pad. A radio-frequency probe may also be bipolar; that is, current travels between two spaced conductor bands on the probe. There may be multiple spaced bands disposed on the probe to promote current conduction between adjacent bands. Microwave energy may be emitted from a microwave antenna placed in the distal probe. The emitted microwave energy may heat tissue in proximity to the antenna, in contrast to radio frequency probes which must make contact with tissue to cause heating. Ultrasound probes incorporate a transducer in the probe that converts electrical signals into ultrasonic energy that vibrates cells in tissue to generate heat. Probes may contain fiberoptic cables to carry laser light to tissue for heating. Light in the infrared region may also be transmitted through fiberoptics to heat cardiac tissue. A flexible sheath 281 attached to the backbone 283 may house a conduit for tissue ablating-energy, and the sheath may be relatively movable with respect to the backbone along a captivating track, as illustrated in FIG. 40C, for enhanced ability to manipulate the ablating instrument into proper position. The tissue-ablating energy may then be supplied via a distributed electrical heater element, or a distributed electrode for RF electrical energy, or an infrared conduit, or a microwave instrument in conventional manner (see, for example, U.S. Pat. No. 6,383,182).

In the configuration of the instrument, as illustrated in FIGS. 40A-C, the sheath 281 containing one or other such tissue-ablating mechanisms may be positioned as previously described and oriented toward cardiac tissue within the intrapericardial space along the entire path 263, 269. The active, tissue-ablating segment need not be longer than approximately the distance along the portion of the path 263, 269 of insertion around the set of four pulmonary veins. Alternatively, the tissue-ablating segment of the instrument may be substantially shorter than the path 263, 269 around the pulmonary veins and may be applied serially along the path 263, 269 to ablate tissue along the entire path. Following application of tissue-ablating energy along the path 263, 269, the tissue-ablating instrument may be withdrawn from the patient's body.

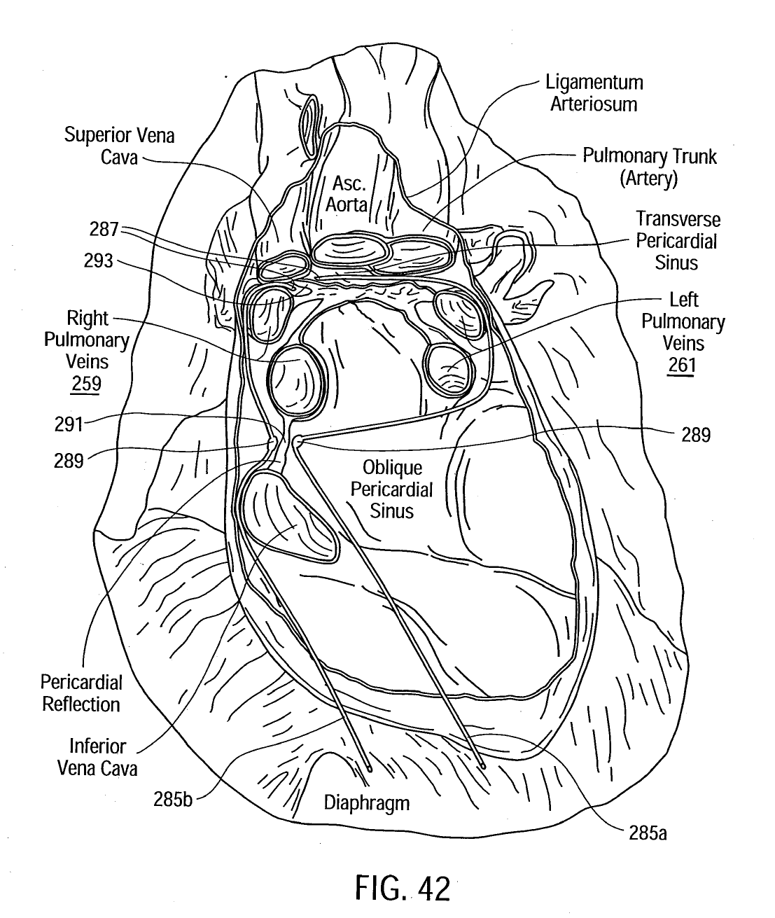

Referring now to FIG. 41, there is shown another embodiment of a tissue-ablating probe in accordance with the present invention in which a flexible elongated body 285, for example, as illustrated and described above with reference to FIGS. 40A-C, also includes magnetic components 287, 289 at the distal end and at a location proximal the distal end for selectively positioning a pair of such tissue-ablating probes along paths, as shown in FIG. 42. Portions of the ablation probes 285 proximal the magnetic bands 289 may include thermally insulating sheaths, for example, to limit exposure of cardiac tissue to RF heating energy only along the portions of the probes 285 intermediate the tips 287 and bands 289. It is desirable to conduct the tissue-ablating procedure from the subxiphoid access site to avoid multiple incisions in a patient's chest, either as thoracotomy incisions or thorascopic incisions.

To encircle the pulmonary veins within the intrapericardial space, as shown in FIG. 42, two tissue-ablating probes 285a, 285b may be advanced along the posterior pericardial surface within the intrapericardial space. One probe 285a may be advanced along the left lateral aspect of the pericardium, track superior to the left superior pulmonary vein, and enter the transverse pericardial sinus. The transverse sinus ends near the right superior pulmonary vein. Inferiorly, the probe 285a may track inferior to the left inferior pulmonary vein, transversely across the oblique pericardial sinus toward the right inferior pulmonary vein, where the probe encounters a pericardial reflection 291 extending between the right inferior pulmonary vein and the inferior vena cava. The second probe 285b is advanced along the right lateral aspect of the pericardium, tracking lateral to the inferior vena cava, right inferior pulmonary vein, and right superior pulmonary vein. The probe 285b tracks superior to the right superior pulmonary vein, until its tip rests close to the tip of the probe 285a in the transverse sinus. A reflection 293 of the pericardium lies along the back of the superior vena cava, and this fold of pericardium separates the tips 287 of the two probes 285a, 285b.

In order to form a substantially continuous ring of ablated tissue surrounding the pulmonary veins, it is desirable to have the tips 287 of the probes 285a, 285b nearly touch each other, although they are separated by a pericardial reflection 293. The distal tips 287 of the probes 285a, 285b contain magnets of opposite polarity to cause the probes to align themselves via magnetic attraction on opposite sides of the pericardial reflection 293 that separates the tips 287. Additionally, the magnetic bands 289 on the ablating probes 285a, 285b substantially align through the pericardial reflection 291 due to the attractive magnetic forces involved. The magnetic bands 289 may be adjusted along the lengths of the probes 285a, 285b to accommodate the patient's anatomy in positioning the magnets properly in close proximity.

The probes 285a, 285b may be formed with resilience and with a predetermined bend, and be retained in straightened-out configuration by a rigid outer sheath that facilitates positioning the probe around corners and into the transverse sinus. For example, the probe 285a may have a preformed ninety-degree bend several centimeters proximal to its distal tip. The probe is inserted through a straight, rigid cannula, and advanced through the operating channel of the endoscopic subxiphoid cannula. The probe 285a is positioned superior to the left superior pulmonary vein, and the cannula retracted to allow the probe to bend and enter the transverse sinus. The probe 285a is advanced further and fully into the transverse sinus. Alternatively, the probe 285a may have an inner lumen that accepts a bent stylet which is inserted into the probe whenever a bend in the probe is desired. A relatively rigid, straight outer sheath may also be used in combination with an inner bent stylet. Specifically, as the bent stylet, which is initially retracted out of the probe 285a, is advanced distally into the probe 285A, the portion of the probe 285a that lies distal to the rigid, straight outer sheath will take the shape of the bent stylet.

The ablation probe 285a, 285b is flexible. A variety of energy sources may achieve ablation of cardiac tissue; e.g. radio frequency, microwave, ultrasound, laser radiation, infrared illumination, and the like. For example, the distal portions of the ablation probes contain a section that emits tissue-ablating energy. The supplied energy at various wavelengths heats cardiac tissue. Radio-frequency energy may be monopolar, that is, the current supplied via the probe travels through the patient's body to a cutaneous grounding pad. A radio-frequency probe may also be bipolar; that is, current travels between two spaced conductor bands on the probe. There may be multiple spaced bands disposed on the probe to promote current conduction between adjacent bands. Microwave energy may be emitted from a microwave antenna placed in the distal probe. The emitted microwave energy may heat tissue in proximity to the antenna, in contrast to radio frequency probes which must make contact with tissue to cause heating. Ultrasound probes incorporate a transducer in the probe that converts electrical signals into ultrasonic energy that vibrates cells in tissue to generate heat. Probes may contain fiberoptic cables to carry laser light to tissue for heating. Light in the infrared region may also be transmitted through fiberoptics to heat cardiac tissue. The ablation probe is flexible and may have various controllable mechanical characteristics, for example, as previously described herein with reference to FIGS. 40A-C. In another embodiment, as illustrated and described herein with reference to FIG. 53, a braided structure 375 forms the length of the probe body 285. A magnetic band 289 is selectively located at axial positions along the probe as desired, for example, by using a pair of endoscopic graspers inserted through an instrument channel in the subxiphoid endoscopic cannula to slide the band 289 along the probe to a selected position. The magnetic band 289 on each probe 285a, 285b may be moved in this manner to positions aligned with the common site directly under the right inferior pulmonary vein to magnetically draw the probes together across the pericardial reflection between the right inferior pulmonary vein and the inferior vena cava, as shown in FIG. 42. Helical tacks or barbs can be located at the tips 287 of the probes to temporarily anchor the probes at the location 293 adjacent the pericardial reflection.

In the treatment of chronic atrial fibrillation, it is desirable to ablate the atrial tissue surrounding the four pulmonary veins (i.e., the left and right superior and inferior pulmonary veins). An ablation probe may be used to ablate the atrial tissue surrounding all four pulmonary veins in a single circle. Alternatively, the two left pulmonary veins and the two right pulmonary veins may be encircled separately in ablation rings.

In accordance with one embodiment of the present invention, an ablation probe is placed using an endoscopic subxiphoid cardiac access cannula and the anterior pericardium is identified and entered. The subxiphoid cannula is advanced to the lateral border of the superior vena cava within the pericardium. A small, 2 cm incision is made in the right chest, at approximately the 5^{th} intercostal space and the anterior axillary line. A second endoscopic cannula is advanced into the right pleural cavity to dissect the tracts posterior to the superior and inferior vena cavae. A light source supplying the endoscopic cannula in the right pleural cavity may be dimmed or extinguished to allow light from the subxiphoid endoscopic cannula to transilluminate through the pericardial and pleural layers to mark the correct spot for vena caval dissection. The pericardial entry instrument may be used to grasp and enter through the pleural and pericardial layers. Following dissection of a tract posterior to the superior vena cava, the ablation probe may be advanced from the right pleural cavity through the dissected tract into the transverse pericardial sinus and lateral to the left pulmonary veins. A grasping instrument may be advanced through the subxiphoid endoscopic cannula to grasp the probe and pull it into position around the pulmonary veins. The subxiphoid endoscopic cannula is then advanced to the lateral border of the inferior vena cava, and the endoscopic cannula in the right pleura cavity is used to dissect a tract posterior to the inferior vena cava, using the transilluminated light from the subxiphoid endoscopic cannula to pinpoint the location of the dissection tract. Following dissection of the tract posterior to the inferior vena cava, the pericardial entry instrument used for the dissection may grasp the distal end of the ablation probe, pull it out through the dissected tract and up to the point of entry posterior to the superior vena cava to complete encirclement of all four pulmonary veins.

The ablation probe remains in the same axial orientation along its length. Torsional deflection of a portion of the probe may lead to ablation of unintended tissue adjacent the left atrium, for example, the esophagus. Application of ablation energy to the esophagus may cause perforation and/or necrosis of the esophagus, with subsequent leakage, scarring and stricture. If a flexible ablation probe is used for the procedure, prior insertion of a non-torsional sleeve posterior to the vena cavae and around the pulmonary veins may prevent twisting of the ablation lead. A tubular sleeve 375, as illustrated in FIG. 53, may contain a braided support in its wall that maintains axial alignment of the sleeve along its flexible length. The tubular sleeve 375 contains an off-round cross-section, (e.g., elliptical or rectangular) and the flexible ablation probe has a matching cross-section to prevent the probe from twisting out of axial alignment as it is advanced through the length of the non-torsional tubular sleeve 375. Manipulation with the endoscopic instruments of the separate sleeve 375 through the dissected tracts and around the pulmonary veins is desirable to prevent injury to the ablation probe from the pulling and grasping movements exerted during encirclement of the pulmonary veins. The braided support in the tubular sleeve 375 may be constructed of plastic material (e.g., nylon, polyethylene) to allow transmission of ablation energy through the wall of the tubular sleeve without significant absorption of the energy. If the ablation probe uses a microwave or ultrasonic source, the energy may be transmitted through the tubular sleeve into the myocardium of the heart.

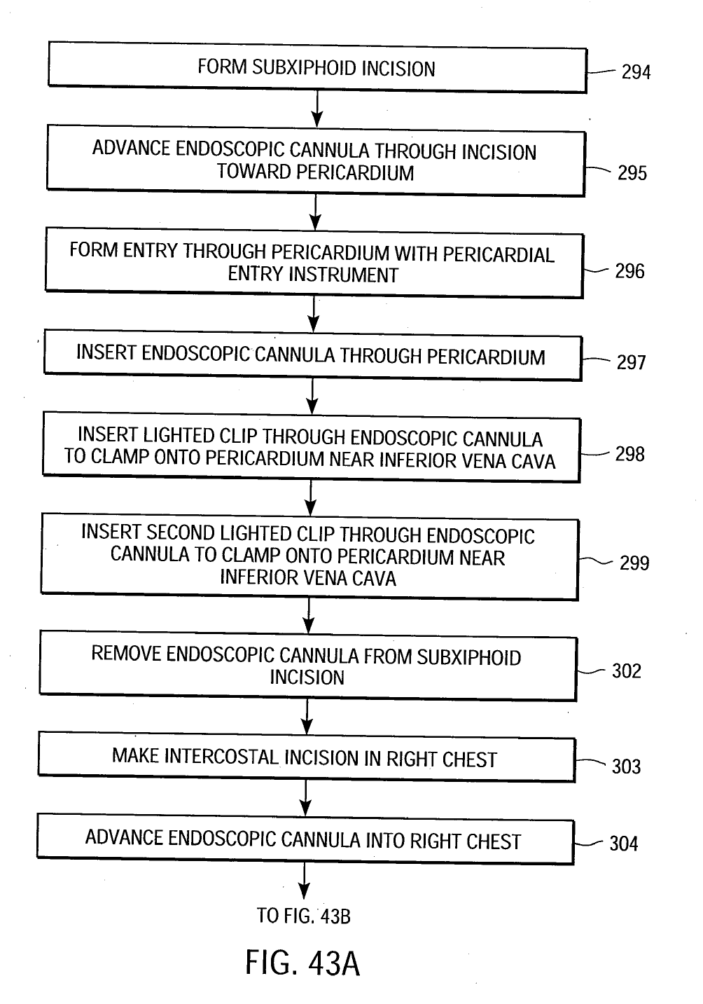

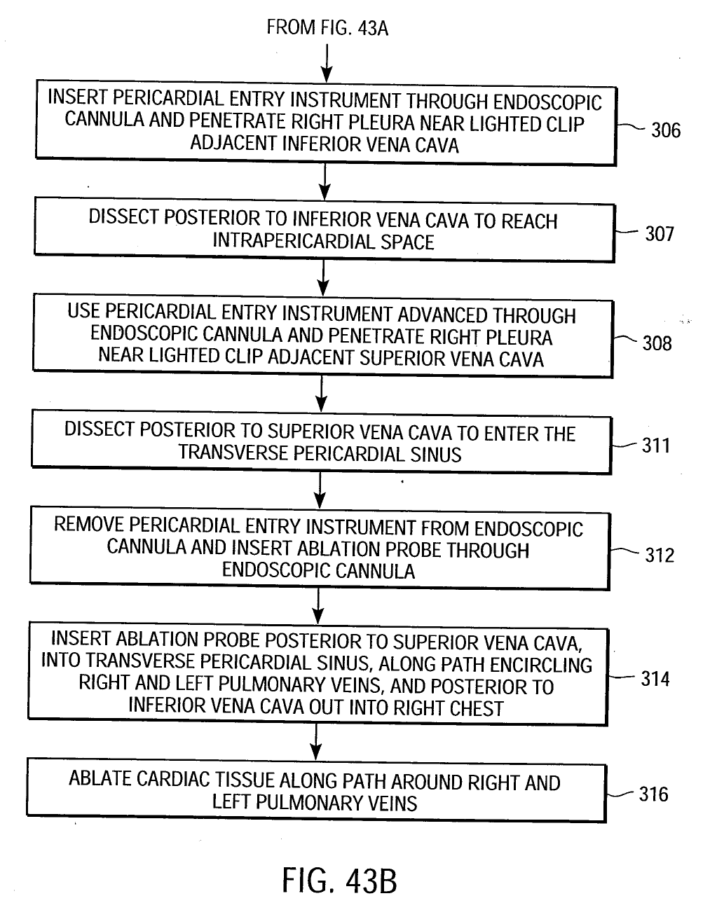

More specifically, the flow chart of FIGS. 43A and 43B illustrates a surgical procedure in accordance with this embodiment of the present invention. A subxiphoid incision is formed 294, and an endoscopic cannula is advanced 295 through the incision and mediastinum toward the pericardium. A pericardial entry instrument is inserted through the endoscopic cannula to form an entry 296 through the pericardium. The endoscopic cannula is inserted through the entry in the pericardium 297. An illumination source is inserted through the endoscopic cannula to attach 298 to the pericardium near the superior vena cava. A second illumination is inserted 299 through the endoscopic cannula to clamp to the pericardium near the inferior vena cava. The endoscopic cannula is removed 302 from the subxiphoid incision. An intercostal incision is made 303 in the right chest. The endoscopic cannula is advanced 304 through the incision into the right chest cavity. The pericardial entry instrument is inserted through the endoscopic cannula and used to penetrate the right pleura 306 near the illumination source adjacent the inferior vena cava. Dissection 307 is conducted posterior to the inferior vena cava to reach the intrapericardial space. The pericardial entry instrument is used through the endoscopic cannula to penetrate the right pleura 308 near the illumination source adjacent the superior vena cava. Dissection 311 is conducted posterior to the superior vena cava to reach the transverse pericardial sinus. The pericardial entry instrument is removed 312 from the endoscopic cannula and the ablation probe is inserted 314 through the endoscopic cannula. The ablation probe is inserted posterior to the superior vena cava into the intrapericardial space in the transverse pericardial sinus, along a path encircling the right and left pulmonary veins, and posterior to the inferior vena cava out into the right chest. The cardiac tissue is ablated 316 along a path around the right and left pulmonary veins to form a transmural lesion along the path.

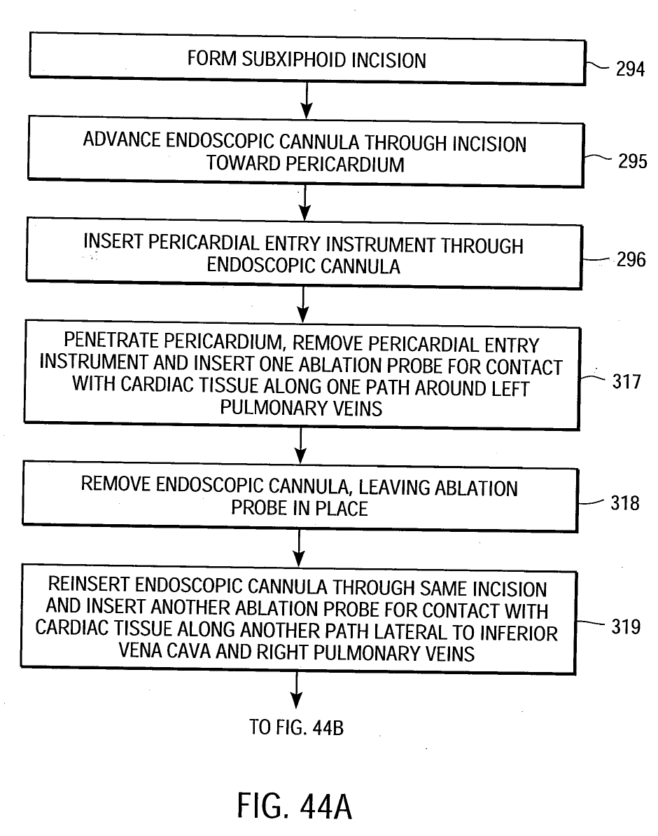

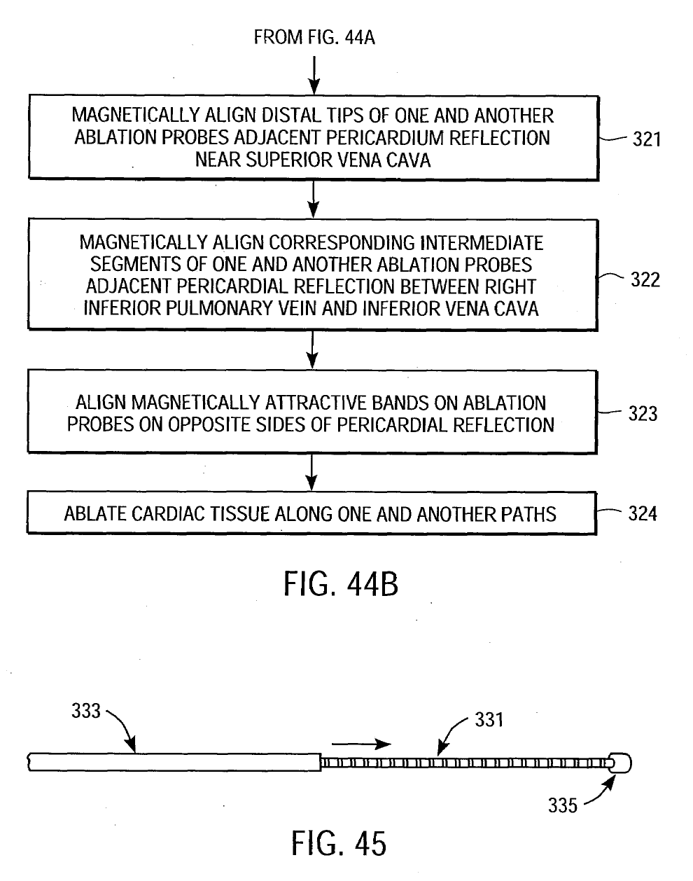

Referring now to FIGS. 44A, 44B, there is shown a flow chart illustrating another surgical procedure in accordance with an embodiment of the present invention. The procedure includes forming a subxiphoid incision 294 and advancing a subxiphoid endoscopic cannula through the incision toward the pericardium 295, in a manner as previously described. A pericardial entry instrument is inserted 296 through a subxiphoid endoscopic cannula and advanced into contact with the pericardium at a location near its anterior apical region. The pericardium is then penetrated, and the entry instrument is removed from the body. An ablation probe is inserted 317 through the endoscopic cannula and into the intrapericardial space. The probe is advanced lateral to the left inferior and left superior pulmonary veins. The opening to the transverse pericardial sinus is visualized superior to the left superior pulmonary vein, through the endoscopic cannula. The probe is advanced into the opening to the transverse pericardial sinus, and is pushed further to the end of the sinus. The tip of this ablation probe extends to the pericardial reflection adjacent the superior vena cava, corresponding to the end of the transverse pericardial sinus. The endoscopic cannula is then removed 318 leaving the probe in the transverse pericardial sinus. The endoscopic cannula is then reinserted through the same subxiphoid incision and same pericardial opening for insertion therethrough of another ablation probe 319 along another path lateral to the inferior vena cava and right, inferior and superior pulmonary veins.

The tips of these ablation probes substantially align 321 on opposite sides of the pericardial reflection adjacent the superior vena cava as a result of magnetic attraction between oppositely-poled magnetic tips. In addition, the one and other ablation probes are manipulated into close proximity 322 along their lengths on opposite sides of the pericardial reflection between the right inferior pulmonary vein and the inferior vena cava. Magnetic bands on each of the ablation probes are located at positions along the respective lengths of the ablation probes to magnetically attract into substantial alignment 323 on opposite sides of the pericardial reflection between the right pulmonary vein and the inferior vena cava. With the associated tips and bands of the ablation probes aligned in close proximity, the ablation probes are then activated 324 to apply tissue-ablating energy to cardiac tissue along the substantially continuous encircling path thus formed by the two ablation probes.

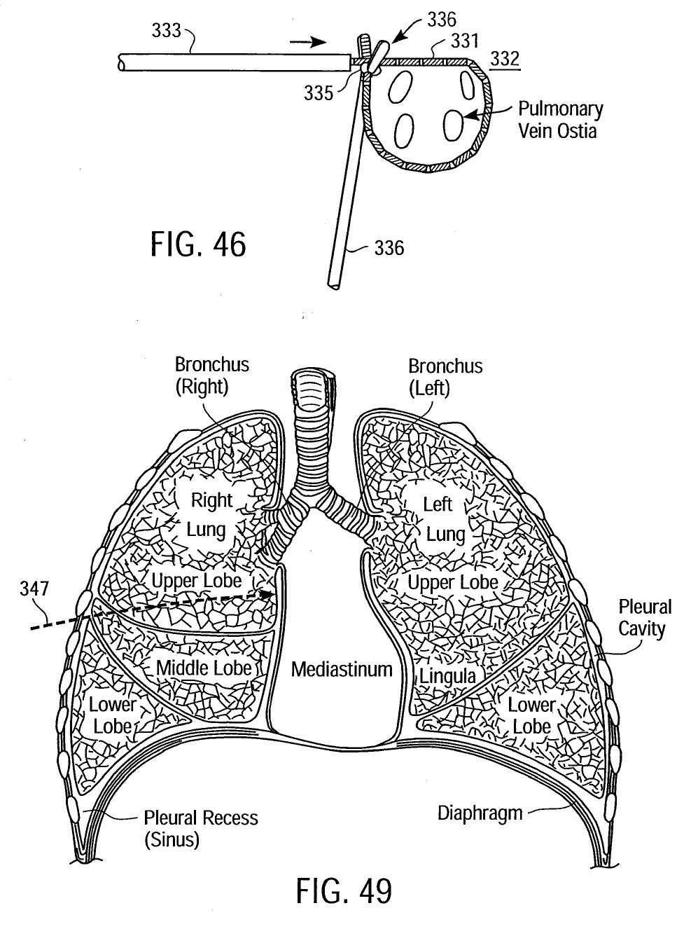

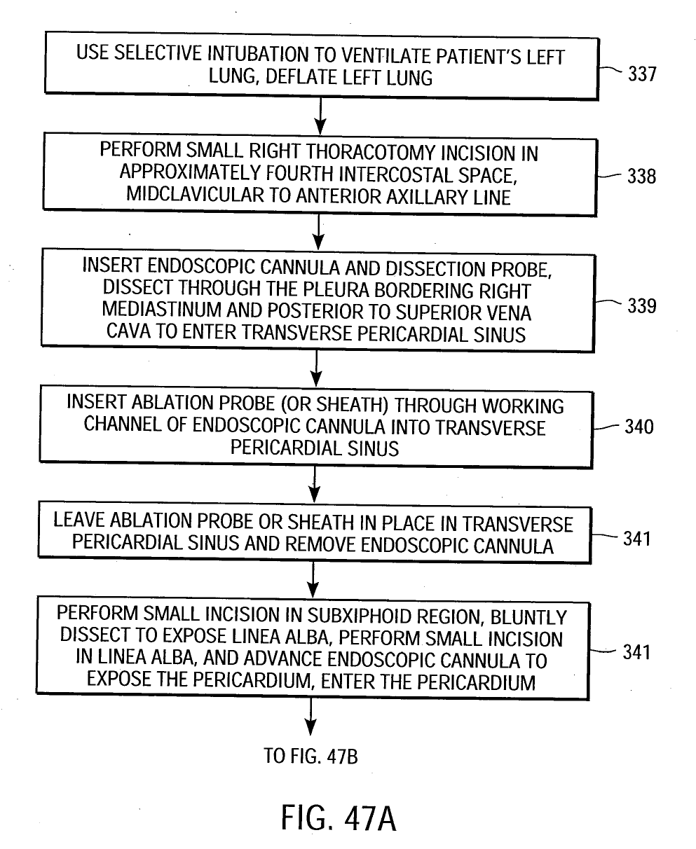

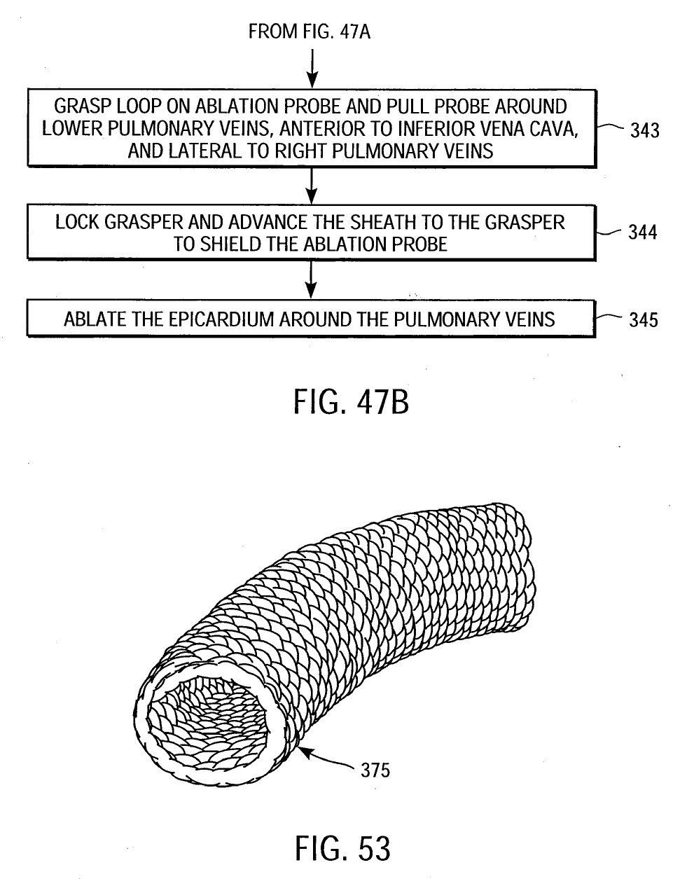

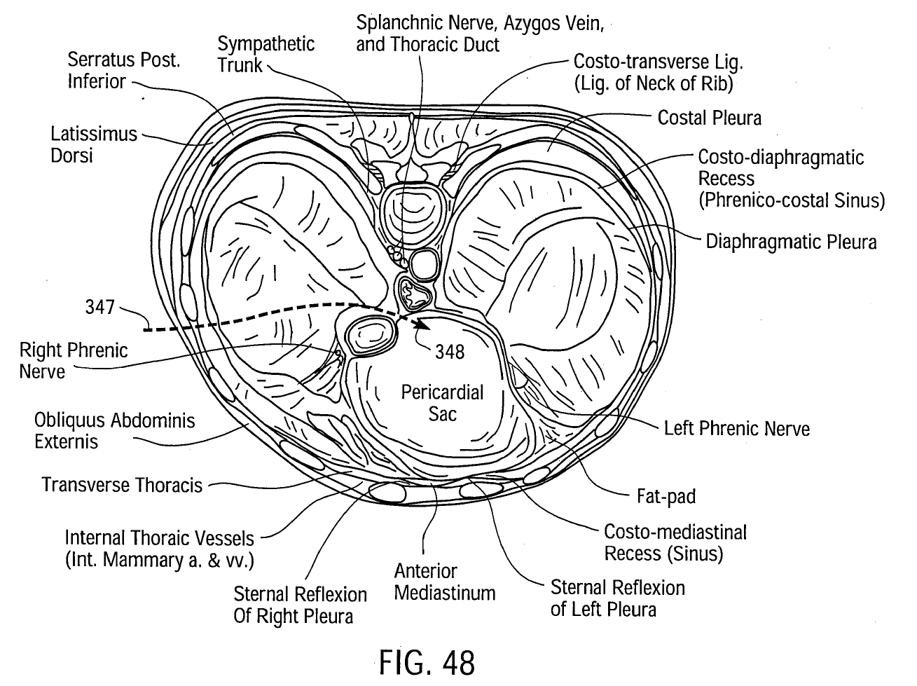

Referring now to FIG. 45, there is shown an ablation probe 331 that is slidable within an insertion sheath 333, and that is laterally flexible at least in one direction but that is torsionally and longitudinally rigid, for example, attributable to a backing structure of tongue and groove segments that are successively pinned or hinged together, as illustrated and described herein with reference to FIGS. 40A, 40B. In this embodiment, the ablation probe 331 includes a suture loop 335 attached at the distal end of the probe 331 to facilitate gripping and pulling of the probe for placement along a path 332 substantially encircling the pulmonary vein ostia, as illustrated in FIG. 46. To position the ablation probe 331 within the intrapericardial space encircling the pulmonary vein ostia, a surgical procedure is performed as illustrated in the flow chart of FIGS. 47A, 47B. Initially, the patient is prepared for surgery and selective intubation is installed to ventilate the patient's left lung 337. The patient's right lung is deflated, and a small right thoracotomy incision is performed 338 on the fourth intercostal space approximately mid-clavicular to the anterior axillary line, as shown on FIGS. 48 and 49. An endoscopic cannula equipped with a tissue-dissecting probe or tip is inserted into the incision to dissect through the pleura 339 bordering the right mediastinum and posterior to the superior vena cava in preparation for entering the transverse pericardial sinus. The ablation probe (or a sheath therefor) is inserted 340 through a working channel in the endoscopic cannula and into the transverse pericardial sinus. A distal end of the ablation probe (or of the sheath therefor) is left in place in the transverse pericardial sinus as the endoscopic cannula is removed 341 back through the dissected channel, leaving the ablation probe (or sheath therefor) in place.

Then, a small incision is formed in the subxiphoid area and tissue is bluntly dissected to expose the linea alba. An incision is made in the linea alba in order to advance 342 the endoscopic cannula posterior to the sternum toward the pericardium. The pericardium is penetrated and a grasping instrument is inserted through the working channel in the endoscopic cannula and into the intra-pericardial space to grasp the loop 335 on the distal tip of the ablation probe 331 and pull the probe laterally around the left pulmonary veins 343 to a level below the left inferior pulmonary vein.

The loop 335 on the tip of the ablation probe 331 is then grasped and pulled across the oblique pericardial sinus toward the right border of the pericardium, anterior to the inferior vena cava, and then upwardly lateral to the right pulmonary veins toward the ablation probe at its entrance into the transverse pericardial sinus. The grasper 336 may now orient the tip 335 of the ablation probe 331 in proximity to the portion of the probe at its entrance into the transverse pericardial sinus in a configuration, as illustrated in FIG. 46. The grasper 336 may be locked to retain the distal end and the entry position of the ablation probe 331 substantially in contact 344 as the sheath 333 of thermally and electrically insulating material is advanced over the ablation probe 331 toward the grasper to thermally and electrically shield the portion of the ablation probe 331 that extends from the grasper 336 toward the intercostal incision 338. With the ablation probe 331 encircling the left and right pulmonary veins substantially as shown in top view in FIG. 46 and oriented toward cardiac tissue within the intrapericardial space, the ablation probe may then be energized, for example, by application thereto of RF or microwave electrical signal or other tissue-ablating energy, to ablate the epicardium 345 to create a transmural lesion in the endocardium around the pulmonary veins. Thereafter, the grasper 336 is unlocked and the ablation probe 331 is removed from around the pulmonary veins, and the incisions performed during the surgical procedure are sutured.

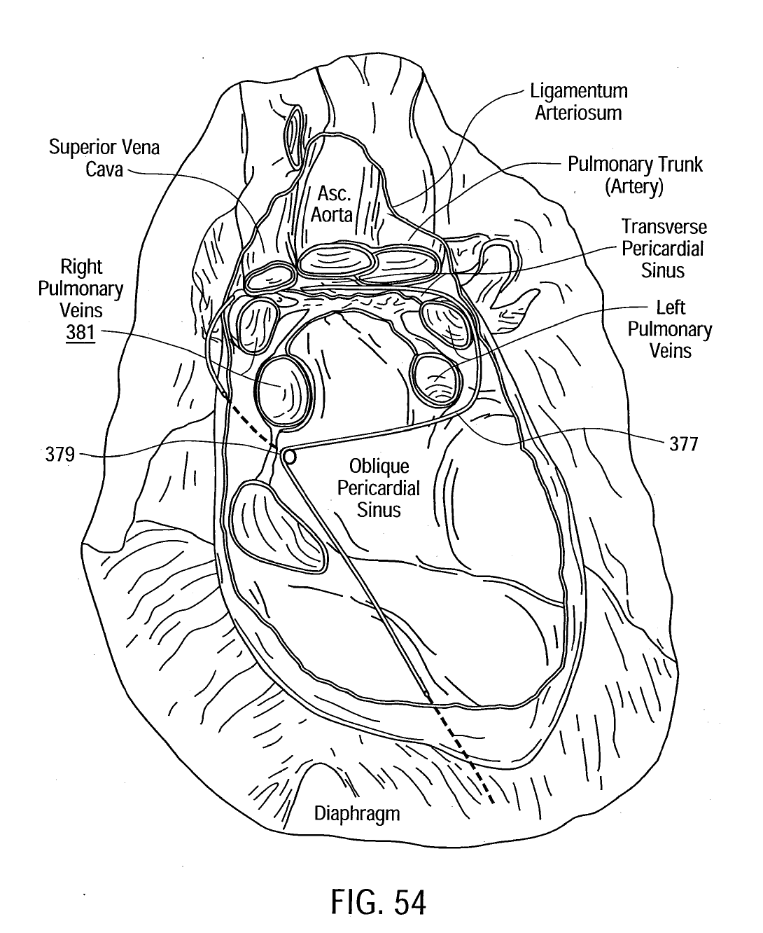

In another embodiment of the present invention, ablation of atrial tissue surrounding the four pulmonary veins may be accomplished using a combined intrapericardial and extrapericardial technique. First, a subxiphoid incision and subsequent procedures, as previously described herein, are used to gain access to and entry into the pericardium at an anterior pericardial entry point. An ablation probe is advanced into the transverse pericardial sinus along path 377 to its termination near the right superior pulmonary vein, as illustrated in FIG. 54. The probe tip lies at the end of the transverse sinus, while its body encircles the four pulmonary veins on three sides, i.e., (1) superior to the superior pulmonary veins, (2) lateral to the left superior and left inferior pulmonary veins, and (3) inferior to the inferior pulmonary veins. This leaves the one side to be completed that is lateral to the right superior and right inferior pulmonary veins.

Dissection lateral to the right superior and right inferior pulmonary veins is hazardous due to the presence of the vena cava. Puncture or laceration of this large diameter, thin walled vessel is dangerous in a closed chest, endoscopic situation, as there is limited access to control hemorrhage. An extrapericardial approach avoids dissection of the vena cava and utilizes a tissue plane directly posterior and lateral to the right superior and right inferior pulmonary veins. Tissue-ablating energy is applied through the posterior pericardium, onto the atrial tissue lateral to the right superior and inferior pulmonary veins. The endoscopic subxiphoid cannula and pericardial entry instrument, as previously described herein, are used to enter the posterior pericardium 379 and dissect an extrapericardial plane lateral to the right pulmonary veins. The inferior vena cava and right inferior pulmonary vein are visualized by the endoscopic subxiphoid cannula, and the pericardial entry instrument is used to grasp the posterior pericardium medial to the inferior vena cava and lateral and inferior to the right inferior pulmonary vein. A small opening is formed by the pericardial entry instrument, and the endoscopic subxiphoid cannula is advanced through this opening in a superior direction, until an extrapericardial tract is formed 381 lateral to the right pulmonary veins, extending from below the right inferior pulmonary vein to above the right superior pulmonary vein. An ablation probe may be advanced into this tract and oriented towards the atrial tissue lateral to the right pulmonary veins.

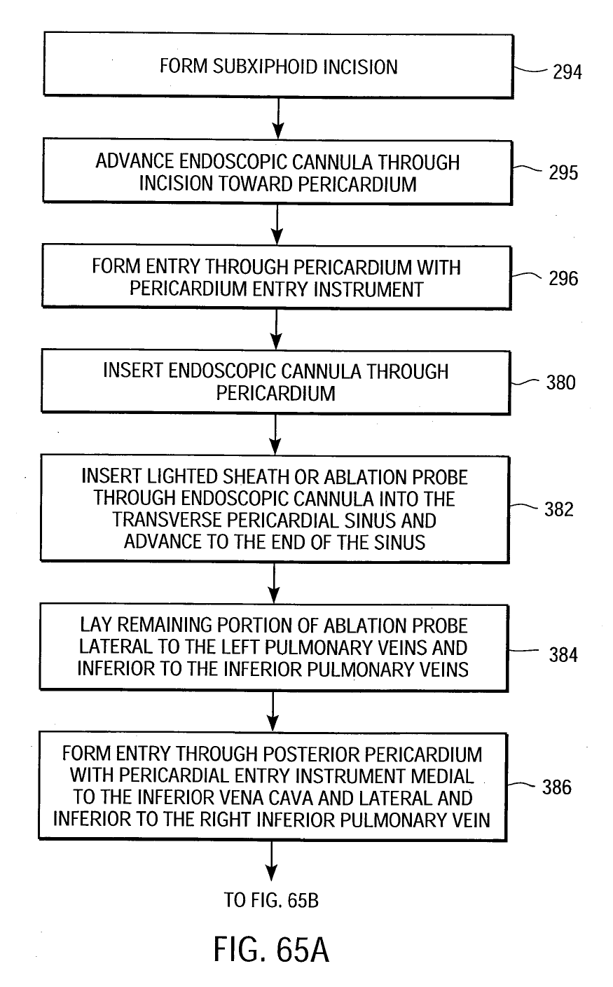

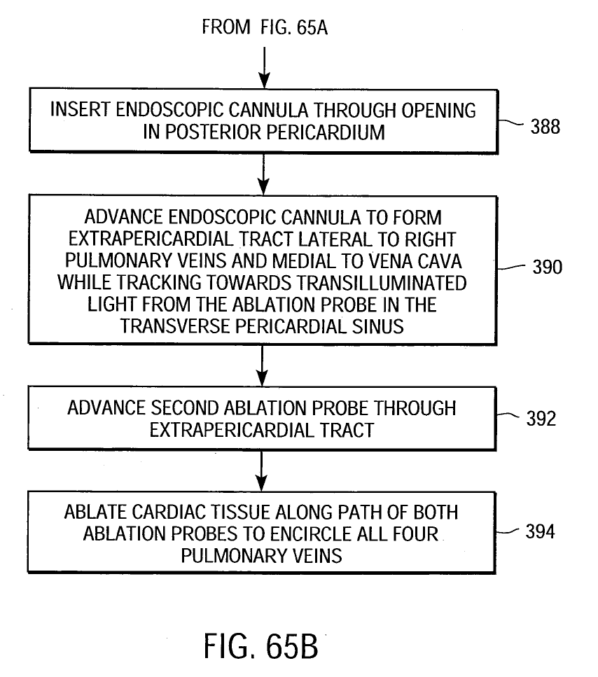

Referring now to the flow chart of FIGS. 65A and 65B, there is disclosed a procedure in accordance with a method embodiment of the present invention for dissecting the extrapericardial tract using the endoscopic subxiphoid cannula and a lighted indicator previously positioned at the end of the transverse pericardial sinus. Specifically, after forming a subxiphoid incision 294 and advancing an endoscopic cannula through the incision toward the pericardium 295, the pericardium is entered 296 using a pericardium entry instrument in the manner as previously described. The endoscopic cannula is inserted through the pericardium 380. A lighted sheath or ablation probe is inserted through the instrument channel of the endoscopic cannula 382 into the transverse pericardial sinus, and is advanced to the end of the sinus. The remaining portion of the ablation probe is positioned 384 lateral to the left pulmonary veins and inferior to the inferior pulmonary veins. Then, the pericardial entry instrument is used 386 to form a posterior pericardial entry point 379 (in FIG. 54) at a location that is medial to the inferior vena cava and lateral and inferior to the right inferior pulmonary vein. An endoscopic cannula is then inserted 388 through the posterior pericardial entry opening and advanced superiorly to form 390 the extrapericardial tract 381 lateral to the right pulmonary veins and medial to the vena cava, as shown in FIG. 54. Formation of this extrapericardial tract is greatly facilitated by advancing toward the light from the ablation probe (or other light source) previously positioned 382 at the end of the transverse pericardial sinus. The light transilluminates through the posterior pericardium to provide an indicator guiding the advancement of the endoscopic subxiphoid cannula as it dissects superiorly from the right inferior pulmonary vein to the right superior pulmonary vein. The indicator light may be attached to a sheath that allows an ablation probe to be advanced inside its lumen. The light may also be attached to the tip of the ablation probe itself. In this embodiment, the ablation probe with a lighted tip is advanced to the end of the transverse pericardial sinus, and a second ablation probe is advanced along the extrapericardial tract to meet up with and align with the lighted ablation probe. Specifically, the second ablation probe can be advanced 392 through the extrapericardial tract thus formed to substantially encircle 394 the four pulmonary veins with the ablation probes positioned intrapericardially and extrapericardially in the manner as described.

FIG. 54 shows the path 377 of the intrapericardial ablation probe, with the tip of the probe residing in the end of the transverse pericardial sinus, and the trailing portion of the probe coursing lateral to the left superior and left inferior pulmonary veins, then inferior to the inferior pulmonary veins. The extrapericardial tract 381 extends lateral to the right pulmonary veins, coursing from the entry point 379 in the posterior pericardium to a position above the right superior pulmonary vein. Addition of the tract 381 and the tract 377 illustrates the combined intrapericardial and extrapericardial ablation lines that surround the four pulmonary veins. With ablation probes thus positioned, application of tissue-ablating energy to the probes completes the ablation of tissue substantially surrounding the four pulmonary veins.

Therefore, ablation of cardiac tissue within the intrapericardial space substantially surrounding the four pulmonary veins as a treatment for chronic atrial fibrillation is greatly facilitated by a tissue-ablating probe, or probes, of the present invention inserted along a tissue-dissected path and manipulated through an endoscopic cannula that is introduced along a dissected working channel from a subxiphoid or intercostal incision. Additionally, suction-oriented instruments facilitate temporary attachment of an elongated body having a working channel therethrough to implement surgical procedures on the suction attached organ at precise locations thereon.

What is claimed is:

1. A surgical instrument comprising:
an elongated body having distal and proximal ends and a lumen therein;
a shaft slidably disposed within the lumen and having a proximal end extending beyond the proximal end of the body to facilitate movement of the shaft relative to the body;
an end effector disposed at the distal end of the shaft; and
a structure for supplying luminous flux to the end effector for illuminating tissue.

2. The surgical instrument according to claim 1 in which the end effector includes a clip including effector elements disposed to transition between open and closed configurations.

3. The surgical instrument as in claim 2 in which the distal end of the elongated body is disposed to overlay the clip at the distal end of the shaft for confining the effector elements in closed configuration, and is disposed to retract relative to the shaft from overlaying the clip for releasing the effector elements to resiliently return to the open configuration.

4. The surgical instrument as in claim 1 in which the structure includes a light-emitting diode disposed with respect to the end effector to illuminate tissue.

5. The surgical instrument as in claim 1 in which the structure includes an optical fiber channel including an end disposed in the end effector to supply luminous flux thereat from a remote source of light.

6. The surgical instrument as in claim 1 in which the elongated body and one of the shaft and end effector are slidably engaged to inhibit relative rotation thereof.

7. The surgical instrument as in claim 2 including diametrically-oriented recesses in the distal end of the body to receive therein the effector elements of the clip in the open configuration.

8. The surgical instrument according to claim 1 in which the distal end of the shaft includes apparatus for temporarily attaching to tissue.

9. A surgical instrument comprising:
an elongated body having lateral flexibility and torsional rigidity and including a conduit; and
tissue-ablating apparatus disposed within the conduit for selectively ablating tissue in proximity thereto.

10. The surgical instrument according to claim 9 in which the elongated body includes a plurality of segments hinged together in succession along a portion of the length, and includes the conduit attached thereto to retain a selected axial orientation of the conduit along the length of the elongated body in response to the torsional rigidity thereof.

11. The surgical instrument according to claim 9 in which the conduit includes one of optical and ultrasound and electrical operating characteristics for ablating tissue proximate the conduit in response to corresponding optical or ultrasound or electrical energy supplied thereto.

12. The surgical instrument according to claim 9 including a first magnetic element disposed near a distal end of the body; and
a second magnetic element disposed along the body at a location proximal the distal end, the first and second magnetic element being disposed to attract toward magnetic elements in proximity thereto.

13. A surgical instrument as in claim 12 including a pair of such elongated bodies with first magnetic elements oriented to attract each other across proximate spacings thereof, and including the second magnetic elements disposed to attract each other across proximate spacings thereof.

14. A surgical instrument as in claim 9 in which the tissue-ablating apparatus includes a flexible loop attached to a distal end thereof to facilitate grasping and pulling within a surgical site.

15. The surgical instrument according to claim 9 comprising:
a sheath overlaying the body in sliding relationship thereto for selective relative positioning of the sheath and body.

16. The surgical instrument according to claim 15 in which the sheath is insulative of tissue-ablating energy and the conduit is conductive of tissue-ablating energy for exposing tissue thereto at a surgical site adjacent to a portion of the conduit not covered by the sheath.

17. A surgical procedure comprising the steps for:
forming an incision;
advancing a cannula through the incision toward a target location on the patient's pericardium;
introducing the illuminator through the cannula into contact with the pericardium at the target location;
attaching the illuminator to the pericardium; and
lighting the illuminator.

18. The surgical procedure according to claim 17 in which the illuminator includes a tissue-gripping clip including a set of jaws that are selectably openable and closeable to grip tissue;
and at least one of the jaws includes a source of illumination.

19. The surgical procedure according to claim 17 including:
forming a subxiphoid incision;
advancing an endoscopic cannula through the subxiphoid incision toward the target area on the pericardium;
introducing a pericardium-penetrating instrument through the endoscopic cannula into contact with the pericardium at the target location; and
forming an aperture through the pericardium at the target site to expose cardiac tissue thereat.

20. The surgical procedure according to claim 19 including:
advancing a tissue-ablating probe through the aperture and along a path laterally adjacent the superior and inferior pulmonary veins; and
ablating cardiac tissue along the path.

21. The surgical procedure according to claim 20 including:
extending the path from the aperture located near the superior vena cava, and across the transverse pericardial sinus, and laterally adjacent the left pulmonary veins, and across the oblique pericardial sinus, anterior to the inferior vena cava and lateral to the right pulmonary veins.

22. The surgical procedure according to claim 20 including:
extending the path for one tissue-ablating probe laterally along the right pulmonary veins and inferior vena cava to a terminus for a distal end of the probe in a pericardium reflection adjacent the superior vena cava;
advancing another tissue-ablating probe along a path across the oblique pericardial sinus, and laterally adjacent the left pulmonary veins and across the transverse pericardial sinus to a terminus for a distal end of said another probe at said pericardial reflection near the superior vena cava;
magnetically attracting the distal ends of said one probe and said another probe into substantial alignment on opposite sides of said pericardial reflection; and
ablating cardiac tissue along said one and said another paths.

23. The surgical procedure according to claim 22 including routing said one path and said another path for said one and said another tissue-ablating probes in close proximity on opposite sides of another pericardial reflection between the inferior right pulmonary vein and the inferior vena cava; and
magnetically attracting adjacent segments of said one tissue-ablating probe and said another tissue-ablating probe into substantial alignment on opposite sides of said another pericardial reflection.

24. The surgical procedure according to claim 23 including:
selectively positioning a magnetically attractive element at least along the length of one of the tissue-ablating probes to substantially align said segments of said one and said another probes on opposite sides of said another pericardial reflection.

25. A surgical procedure comprising the steps for:
forming an incision;
advancing an endoscopic cannula through the incision toward a target location on the patient's pericardium;
introducing a pericardium-penetrating instrument through the endoscopic cannula into contact with the pericardium at the target location;
forming an aperture through the pericardium at the target site to expose cardiac tissue thereat;
advancing a tissue-ablating probe through the aperture and along a path within the intrapericardial space laterally adjacent a pulmonary vein; and
energizing the probe to ablate cardiac tissue.

26. The surgical procedure according to claim 25 further comprising the steps for:
advancing the tissue-ablating probe along the path within the intrapericardial space inferior to the inferior pulmonary veins and lateral to the left inferior and left superior pulmonary veins into the transverse pericardial sinus near the superior vena cava;
forming a posterior pericardial entry at a location intermediate the right inferior pulmonary vein and the inferior vena cava;
advancing a second ablation probe from the posterior pericardial entry lateral to the right pulmonary veins to a location superior to the right superior pulmonary vein near the tissue-ablating probe in the transverse pericardial sinus; and
energizing the tissue-ablating probe and the second tissue-ablating probe to ablate cardiac tissue along the courses thereof.

27. The surgical procedure according to claim 25 further comprising the steps for:
illuminating at least a portion of the tissue-ablating probe positioned within the transverse pericardial sinus; and
visualizing the advancement of the second ablation probe toward tissue illuminated by the illuminated portion of the tissue-ablating probe positioned within the transverse pericardial sinus.

28. The surgical procedure according to claim 25 in which advancing the tissue-ablating probe along a path includes laterally adjacent the superior and inferior pulmonary veins.

29. The surgical procedure according to claim 25 in which the probe is advanced along the path extending from the aperture located near the superior vena cava, and across the transverse pericardium sinus, and laterally adjacent the left pulmonary veins, and across the oblique pericardial sinus, anterior to the inferior vena cava and lateral to the right pulmonary veins.

30. The surgical procedure according to claim 20 including:
extending the path for one tissue-ablating probe laterally along the right pulmonary veins and inferior vena cava to a terminus for a distal end of the probe in a pericardium reflection adjacent the superior vena cava;
advancing another tissue-ablating probe along a path across the oblique pericardial sinus, and laterally adjacent the left pulmonary veins and across the transverse pericardial sinus to a terminus for a distal end of said another probe at said pericardial reflection near the superior vena cava;
magnetically attracting the distal ends of said one probe and said another probe into substantial alignment on opposite sides of said pericardial reflection; and
ablating cardiac tissue along said one and said another paths.

31. The surgical procedure according to claim 30 including routing said one path and said another path for said one probe and said another probe in close proximity on opposite sides of another pericardial reflection between the inferior right pulmonary vein and the inferior vena cava; and
magnetically attracting adjacent segments of said one probe and said another probe into substantial alignment on opposite sides of said another pericardial reflection.

32. The surgical procedure according to claim 31 including:
selectively positioning a magnetically attractive element at least along the length of one of the probes to substantially align said segments of said one and said another probes on opposite sides of said another pericardial reflection.

33. A surgical procedure comprising the steps for:
forming an intercostal thoracotomy;
inserting an endoscopic cannula through the thoracotomy and penetrating tissue along a path toward the pericardium;
forming an aperture through the pericardium at a location near the superior vena cava;
inserting a structure including an elongated body through the endoscopic cannula and aperture along a path traversing the transverse pericardial sinus;
forming a subxiphoid incision;
inserting an endoscopic cannula through the subxiphoid incision toward the pericardium;
forming another aperture in the pericardium at a location near the apex;
inserting a grasping instrument through the endoscopic cannula within the subxiphoid incision and through said another aperture to grasp a distal tip of the elongated body to extend the path thereof laterally along the left pulmonary veins;
grasping the distal end of the elongated body and extending the path thereof laterally of the right pulmonary veins substantially to the location of entry of the elongated body into the transverse pericardial sinus to substantially encircle the pulmonary vein ostia with the elongated body.

34. The surgical procedure according to claim 33 in which the elongated body includes a sheath slidably overlaying a tissue-ablating probe and includes:
relatively slidably positioning the sheath and probe to expose the probe without overlaying sheath substantially encircling the pulmonary vein ostia.

35. The surgical procedure according to claim 32 in which the probe includes an energy-transmissive conduit for ablating tissue adjacent thereto in response to tissue-ablating energy applied to the conduit.

36. The surgical procedure according to claim 35 in which the energy transmissive conduit is positioned adjacent epicardial tissue along the encircling path for the ablation thereof in response to applied tissue-ablating energy signal.

37. A surgical procedure comprising the steps for:
forming an incision;
advancing an endoscopic cannula through the incision toward a target location on a patient's pericardium;
introducing a pericardium-penetrating instrument through the endoscopic cannula into contact with the pericardium at the target location;
forming an aperture through the pericardium at the target site to expose epicardial tissue;
introducing a tacking instrument through the endoscopic cannula through the aperture in the pericardium for installing a plural number of tacks at selected spaced locations in the epicardial tissue; and
installing an element in contact with at least a pair of the plural number of tacks to exert tension thereon.

38. The surgical procedure according to claim 37 in which the incision is a subxiphoid incision.

39. The surgical procedure according to claim 37 in which the tacking instrument is manipulated to install one epicardial tack at the region of the mitral annulus inferior to the circumflex coronary artery, and another epicardial tack at the region of the mitral annulus inferior to the coronary sinus.

40. The surgical procedure according to claim 37 in which the element includes a strand attached to each of the installed epicardial tacks in tension therebetween.

41. The surgical procedure according to claim 40 in which the strand is a suture.

42. The surgical procedure according to claim 40 in which the strand is a band or belt.

43. The surgical procedure according to claim 37 in which installing includes:
assembling a suture with a pair of loops formed with slip knots having trailing suture ends at spaced locations along the length of the suture;
positioning one of the pair of loops of the suture about one of the plural number of installed epicardial tacks;
positioning another of the pair of loops of the suture about another of the plural number of installed epicardial tacks; and
tensioning the trailing suture ends to tension the suture between the pair of loops disposed about the installed epicardial tacks.

44. The surgical procedure according to claim 43 in which tensioning includes advancing an elongated hollow tube along a trailing suture end from a slip knot for engagement thereof with a distal end of the tube; and
pulling on the trailing suture end relative to the tube to selectively decrease a suture loop about an installed epicardial tack.

45. The surgical procedure according to claim 39 including:
installing an additional number of epicardial tacks intermediate said one and another tacks; and
installing elements in tension between at least pairs of the number of installed epicardial tacks.

46. The surgical procedure according to claim 43 including:
attaching clips to the trailing suture ends to inhibit slip thereof through the knots.

47. The surgical procedure according to claim 46 including:
introducing a clip-applying instrument through the endoscopic cannula and attaching a clip to a trailing suture end adjacent the corresponding slip knot; and
trimming the trailing suture end remote from the clip attached thereof.

48. A surgical procedure on the heart comprising the steps for:
forming a subxiphoid incision;
advancing through the subxiphoid incision toward a target site on the pericardium an endoscopic cannula having a lumen therethrough;
introducing a pericardium entry instrument through the endoscopic cannula into contact with the pericardium at the target site;
forming an aperture through the pericardium at the target site;
inserting the endoscopic cannula through the aperture formed in the pericardium;
advancing one flexible surgical apparatus through the lumen in the endoscopic cannula into the transverse pericardial sinus toward the end of the sinus;
positioning a portion of the one flexible surgical apparatus along a path lateral to the left pulmonary veins and inferior to the inferior pulmonary veins;
forming an aperture through the posterior pericardium medial to the inferior vena cava and lateral and inferior to the right inferior pulmonary vein:
advancing an endoscopic cannula through the aperture in posterior pericardium to form an extrapericardial tract lateral to the right pulmonary veins and medial to vena cava and toward a region near the end of the transverse pericardial sinus; and
advancing another flexible surgical apparatus through the extra pericardial tract to substantially encircle all pulmonary veins with the one and another flexible surgical apparatuses.

49. The surgical procedure as in claim 48 in which advancing the one flexible surgical apparatus illuminates tissue at least near the end of the transverse pericardial sinus; and in which
advancing an endoscopic cannula through the aperture in posterior pericardium proceeds toward tissue illuminated near the end of the transverse pericardial sinus.

50. The surgical procedure as in claim 48 including the steps for:
positioning one tissue-ablating probe as the one flexible surgical apparatus in the transverse pericardial sinus and along the path lateral to the left pulmonary veins and inferior to the inferior pulmonary veins;
positioning another tissue-ablating probe as said another flexible surgical apparatus in the extrapericardial tract to substantially encircle all pulmonary veins with tissue-ablating probes; and
energizing the tissue-ablating probes to ablate cardiac tissue along paths of the probes.

51. A surgical procedure on the heart comprising the steps for:
forming a subxiphoid incision;
advancing through the subxiphoid incision toward a target site on the pericardium an endoscopic cannula having a lumen therethrough;
introducing a pericardium entry instrument through the lumen in the endoscopic cannula into contact with the pericardium at the target site;
forming an aperture through the pericardium at the target site near the superior vena cava;
advancing one tissue-ablating probe through the aperture along a path laterally along the right pulmonary veins and inferior vena cava to a terminus for a distal end of the one probe in a pericardium reflection adjacent the superior vena cava;
advancing another tissue-ablating probe along a path across the oblique pericardial sinus and laterally adjacent the left pulmonary veins and across the transverse pericardial sinus to a terminus for a distal end of said another probe at said pericardial reflection hear the superior vena cava in substantial alignment with the distal end of the one probe on opposite sides of said pericardial reflection; and
ablating tissue along said one and said another paths.

52. A surgical instrument comprising:
a flexible cannula having distal and proximal ends and at least one lumen therein; and
a tensioning member in the at least one lumen for bending the flexible cannula into a desired shape.

53. The surgical instrument of claim 52 in which the desired shape is formed and maintained in response to tension selectively established in the tensioning member from near the proximal end.

54. Surgical apparatus comprising:
one tissue-ablating probe configured for passage through an aperture in a patient's pericardium near the superior vena cava for positioning along a path laterally of the right pulmonary veins and inferior vena cava to a terminus for a distal end of the one probe in a pericardium reflection adjacent the superior vena cava;
another tissue-ablating probe configured for positioning along a path across the oblique pericardial sinus and laterally adjacent the left pulmonary veins and across the transverse pericardial sinus to a terminus for a distal end of said another probe at said pericardial reflection near the superior vena cava in substantial alignment with the distal end of the one probe on opposite sides of said pericardial reflection;
and the probes ablate tissue along said one and said another paths in response to tissue-ablating energy supplied to the probes.
